(19) United States
(12) Patent Application Publication
(10) Pub. No.: US 2012/0225076 A1
(43) Pub. Date: Sep. 6, 2012

(54) FRA-1 TARGET GENES AS DRUG TARGETS FOR TREATING CANCER

(75) Inventors: Daniel Simon Peeper, Amstelveen (NL); Christophe Jean Claude Desmet, Liege (BE); Fabien Reyal, Paris (FR)

(73) Assignee: Stichting Het Nederlands Kanker Instituut, Amsterdam (NL)

(21) Appl. No.: 13/422,788

(22) Filed: Mar. 16, 2012

Related U.S. Application Data
(63) Continuation-in-part of application No. PCT/NL2010/050594, filed on Sep. 15, 2010.
(60) Provisional application No. 61/242,935, filed on Sep. 16, 2009.

(30) Foreign Application Priority Data
Sep. 17, 2009 (EP) 09170573.1

(51) Int. Cl. A61K 39/395; C40B 30/04; C40B 40/08; A61P 35/04; C07H 21/04; C07H 21/02; C07K 16/18; C12Q 1/68; A61K 31/7088

(52) U.S. Cl. 424/139.1; 435/6.13; 506/9; 506/17; 514/44 A; 536/24.5; 530/387.9

(57) ABSTRACT

The invention relates to the use of an inhibitor of one of the following polypeptides, wherein the polypeptide is represented by the following sequences selected from the following group SEQ ID NO:1-32, each of the polypeptide being preferably as identified in claim 1 as a medicament, preferably for preventing, delaying and/or treating metastasis in a cancer patient. The invention also relates to a diagnostic portfolio comprising or consisting of isolated nucleic acid sequences, their complement or portions thereof, of a combination of genes selected from the groups consisting of genes represented by the following sequences SEQ ID NO:1-32 or SEQ ID NO:1-169.

### RELATED APPLICATIONS

This application is a continuation-in-part of PCT international application Ser. No. PCT/NL2010/050594, filed Sep. 15, 2010, designating the United States, which claims the benefit of U.S. Provisional Application No. 61/242,935, filed Sep. 16, 2009, and claims the benefit of European Application No. 09170573.1, filed Sep. 17, 2009. The entire contents of the aforementioned patent applications are incorporated herein by this reference.

### FIELD OF THE INVENTION

The invention relates to the use of an inhibitor of one of the following polypeptides, wherein the polypeptide is represented by the following sequences selected from the following group SEQ ID NO:1-32, each of the polypeptide being preferably as identified in claim 1 as a medicament, preferably for preventing, delaying and/or treating metastasis in a cancer patient. The invention also relates to an ex vivo method of prognosticating metastasis in a cancer patient comprising identifying differential modulation of a gene (relative to the expression of a same gene in a control) in a combination of genes selected from the groups consisting of genes represented by the following sequences SEQ ID NO:1-169 and/or SEQ ID NO:1-32.

### BACKGROUND OF THE INVENTION

Metastatic spread of tumor cells is a highly complex process in which tumor cells have to overcome multiple barriers and complete all the steps of a so-called “metastatic cascade”. In carcinomas, the most frequent solid tumors that originate from epithelial tissue, these steps involve disruption of normal epithelial cell-cell contacts, breaching of the basement membrane, invasion of the neighboring tissue, intravasation in blood or lymph vessels, transport through the vessels, extravasation and growth at secondary sites (Gupta and Massague, 2006). Several of these steps require the acquisition of cell motility, with disruption of the normal epithelial organization as a prerequisite (Cavallaro and Christofori, 2004). It has often been suggested that these processes involve the hijacking by cancer cells of an embryonic program known as epithelial-to-mesenchymal transition (EMT) in which epithelial cells acquire more flexible and mobile properties reminiscent to those of mesenchymal cells (Thiery and Sleeman, 2006; Yang and Weinberg, 2008). One main feature of EMT is the downregulation of epithelial proteins, most predominantly E-cadherin. In addition, cells also often acquire expression of mesenchymal proteins such as N-cadherin.

Classically, metastasis has been considered a late and rare event in carcinoma progression (Fidler, 2003). Through selection processes or stochastically, some cells in a primary tumor are believed to acquire new alterations that give them the potential to metastasize. More recently, a model proposed that, as a function of the type of mutation driving primary tumorigenesis, some tumors are endowed early on with a proclivity to metastasize (Bernards and Weinberg, 2002). Other alterations occurring later in tumorigenesis would ultimately endow a subset of the tumor cells with full metastatic potential. This idea originates from the observation that gene-expression classifiers based on the genetic make-up of the bulk of primary breast cancer cells can predict tumor recurrence (van de Vijver et al., 2002; Ramaswamy et al., 2003). Consistent with this view, it has recently been shown that some oncogenes that allow escape from failsafe cell cycle programs, a prerequisite of tumorigenesis, simultaneously induce EMT in breast epithelial cells (Ansieau et al., 2008), thereby favoring metastatic dissemination.

Identifying the genes that contribute to the metastatic process is key to the understanding of metastasis, as well as to the development of new therapies. Several cancer therapies had already been developed. However, none of them had been entirely successful. This is why there is still a need for identifying new compounds that could be used for treating cancer.

To identify novel metastasis genes, we previously exploited Rat Intestinal Epithelial (RIE-1) cells to perform a genome-wide screen for suppressors of anoikis (detachment-induced cell death) (Douma et al., 2004). This led to the identification of the neurotrophic receptor tyrosine kinase TrkB, which upon co-expression with its primary ligand BDNF converted parental cells from anoikis-sensitive, non-oncogenic cells into anoikis-resistant, tumorigenic and highly metastatic cells (Douma et al., 2004) (Geiger and Peeper, 2005). As these cells, as well as independently engineered TrkB-expressing Rat Kidney epithelial (RK3E) cells, completely depend on TrkB activity for their oncogenic and metastatic potential, and because this is manifested with very short latencies (Douma et al., 2004 and this paper), we took advantage of these robust cell systems to screen for novel critical metastasis genes.

### DESCRIPTION OF THE INVENTION

Metastatic spread of tumor cells accounts for most of cancer mortality, yet many of its driving mechanisms remain to be elucidated. By combining genetic and functional analysis with RNAi in a metastasis model, we identify here a strict requirement for the transcription factor Fra-1 (Fos-related antigen-1) in tumor cell dissemination. This was associated with a critical role for Fra1 in epithelial-to-mesenchymal transition (EMT), cell migration and invasion, all processes contributing to metastasis. In support of these observations, we demonstrate that Fra-1 depletion from human breast cancer cells suppresses their ability to metastasize from orthotopic primary tumors. Underscoring a key role for Fra-1 in breast cancer metastasis, microarray analysis of Fra-1-depleted breast cancer cells identified a gene-expression signature that predicted tumor recurrence with high accuracy. In addition, we identified 168 gene targets of Fra-1 whose expression is significantly altered in metastasized cells. These genes are identified in Table 2 and any combination thereof could be used as a classifier as explained herein. In addition, among these 168 genes and Fosl1 (encoding Fra1) (i.e. among these 169 genes), we identified 32 genes including Fosl1 as being significantly over-expressed in poor prognosis breast cancer cells. These genes were classified in functional clusters (see Table 5). Given the prevalence of Fra-1 overexpression in breast cancer and other tumors, these data strongly suggest that inhibition of at least one of these genes can be used for treating cancer and especially metastasis. Altogether, the results presented herein indicate that Fra1 and/or some of its downstream effectors may represent valuable targets in preventing, delaying and/or treating metastasis development in cancer, preferably breast cancer.

### Ex Vivo Methods

In a first aspect, there is provided an ex vivo method of prognosticating metastasis in a cancer patient comprising identifying differential modulation of a gene (relative to the expression of a same gene in a baseline) in a combination of genes selected from the groups consisting of genes represented by the following sequences SEQ ID NO:1-169 or SEQ ID NO:1-32.

In the context of the invention, “prognosticating” means either a predictive risk assessment of a cancer patient for metastasing (i.e. predict the presence of metastases in the future, or pre-symptomatic prediction of risk of metastasis) or an assessment of a metastasized cancer in a patient. It may also refer to the likelihood that a patient will respond to a given therapy or to the response of a patient to a therapy he has already been administered. Such a prognostication method is crucial to have since usually once metastasis has been assessed in a cancer patient, his/her chances of survival decrease dramatically.

In the context of the invention, a “patient” may be an animal or a human being. Preferably, a patient is a human being.

In the context of the invention, “metastasis” preferably referred to “metastasis” as assessed in a cancer patient by ultrasound examination of lymph nodes, liver, thorax or any other organ suitable for ultrasound examination, lymph node dissection, scintigraphy of the bones or any other organs suitable for scintigraphy, standard radiography or any other technique suitable for the detection of metastasis. More preferably, “metastasis” refers to the “detection of a metastatic activity” within tumour cells in one of the in vivo animal models as described hereafter. Metastasis can be best studied in vivo in xenograft experiments in mice (nude mice or other suitable mouse strains). Briefly, approximately 1.10⁵ to 1.10⁷ tumour cells are injected either sub-cutaneously (as described in Douma S., et al (2004), Nature, 430:1034-1040), or orthotopically (that is, in the organ or tissue that corresponds to the tissue type of the tumour cells). For example, breast tumour cells are injected into a mammary gland (as described in Erler J. T., et al, (2006), Nature, 440: 1222-1226). Alternatively, cells can be injected directly in the blood circulation of the mice (as described in Erler J. T., et al, (2006), Nature, 440: 1222-1226). The visualisation of at least one visible lesion formed by tumour cells at a site distant from the site of injection reveals a metastatic activity of a tumour cell. Detection of a lesion is usually carried out by microscopic analysis of a series of cross sections of paraffin-embedded tissue. A visible metastatic lesion may comprise at least 4, 6, 8, 10, 12, 14, 15, 17, 19, 20, 22, 24, 25 tumour cells or more. Seeding and growth of metastases will occur at time points depending on the type of tumour cell, typically starting at several days after inoculation, or several weeks or months.

In the context of the invention, a “gene” preferably means a nucleotide acid molecule which is represented by a nucleotide acid sequence and which encodes a protein or polypeptide. A gene may comprise a regulatory region.

In the context of the invention, “a combination of genes selected from the group consisting of genes represented by the following sequences SEQ ID NO:1-32” preferably means:

“A gene or a nucleotide wherein the nucleotide sequence is selected from the groups consisting of:

(1) a nucleotide sequence encoding an enzyme ABHD11, AURKB, CHML, EZH2, FEN1, IGFBP3, PAICS, PCOLN3, PPP2R3A, PTGES, PTP4A1, and SCD,

(2) a nucleotide sequence encoding a transcription factor E2F1, FOSL1, and FOXM1,

(3) a nucleotide sequence encoding a structural protein C22orf18, CHAF1A, H2AFZ, SMTN, TJAP1, D21S2056E,

(4) a nucleotide sequence encoding a receptor ADORA2B;

(5) a nucleotide sequence encoding an adhesion molecule MTDH,

(6) a nucleotide sequence encoding an apoptose inhibitor BIRC5 and PHLDA1

(7) a nucleotide sequence encoding a protein involved in DNA replication/transcription MCM10, MCM2 and TRFP and

(8) a nucleotide sequence encoding a SEC14L1, SFN, SH3GL1 and YTHDF1”.

In the context of the invention, a “cancer” in the expression a “cancer patient” preferably means that a cancer has already been diagnosed in a given patient. Using a method of the invention, metastasis can be prognosticated in any kind of cancer. Preferably, a cancer is such that it is already known to the skilled person that such cancer can potentially lead to metastasis. In another preferred embodiment, a cancer is such that it is technically possible to isolate a sample containing a tumour cell. A cancer may be melanoma, colon, prostate, lung, thyroid, or breast cancer. A preferred cancer is breast cancer.

“Modulated” genes are preferably those that are differentially expressed as up regulated or down regulated in non-normal cells (tumour cells or metastasised tumour cells). Up regulation and down regulation are relative terms meaning that a detectable difference (beyond the contribution of noise in the system used to measure it) is found in the amount of expression of the genes relative to a baseline. In this case, a baseline preferably comes from a pool of non cancer patients, or preferably patients with cancer but without detectable metastasis. A pool of these patients preferably contains 1, 3, 5, 10, 20, 30, 100, 400, 500, 600 or more patients. The expression level of a gene of interest in the non-normal cells is then considered either up regulated or down regulated relative to a baseline level using the same measurement method.

In the context of the use of diagnostic portfolios, a baseline is the measured gene expression of a large pool of cancer patients. Usually, large means at least 50 cancer patients, at least 70, 100, 150, 200, 250, 300, 350, 400, 450, 500, 550, 600 or more. Preferably, the gene expression levels in this large pool of cancer patients is used in this application to generate a good and a poor prognostic centroids as extensively explained in the experimental part in the section entitled “Classifier generation”.

The assessment of the expression level of a gene in order to assess whether a gene is modulated is preferably performed using classical molecular biology techniques to detect mRNA levels, such as (real time) reverse transcriptase PCR (whether quantitative or semi-quantitative), mRNA (micro)array analysis or Northern blot analysis, or other methods to detect RNA. Alternatively, according to another preferred embodiment, in a prognosticating method the expression level of a gene is determined indirectly by quantifying the amount of the polypeptide encoded by said gene. Quantifying a polypeptide amount may be carried out by any known techniques. Preferably, polypeptide amount is quantified by Western blotting. The skilled person will understand that alternatively or in combination with the quantification of an identified gene and/or corresponding polypeptide, the quantification of a substrate of said corresponding polypeptide or of any compound known to be associated with the function of said corresponding polypeptide or the quantification of the function or activity of said corresponding polypeptide using a specific assay is encompassed within the scope of the prognosticating method of the invention. In a preferred embodiment, the assessment of the expression level of a gene is carried out using (micro)arrays as later defined herein.

Since the expression levels of a gene and/or amounts of a corresponding polypeptide may be difficult to be measured in a cancer patient, a sample from a patient is preferably used. According to another preferred embodiment, the expression level (of a gene or polypeptide) is determined ex vivo in a sample obtained from a patient. A sample may be liquid, semi-liquid, semi-solid or solid. A preferred sample comprises 100 or more tumour cells and/or a tumour tissue from a cancer patient to be tested taken in a biopsy. Alternatively and or in combination with earlier preferred embodiment, a sample preferably comprises blood of a patient. The skilled person knows how to isolate and optionally purify RNA and/or protein present in such a sample. In case of RNA, the skilled person may further amplify it using known techniques.

An increase (or up regulation) (which is synonymous with a higher expression level) or decrease (or down regulation) (which is synonymous with a lower expression level) of the expression level of a gene (or steady state level of the encoded polypeptide) is preferably defined as being a detectable change of the expression level of a gene (or steady state level of the encoded polypeptide or any detectable change in the biological activity of the polypeptide) using a method as defined earlier on as compared to the expression level of a corresponding gene (or steady state level of the corresponding encoded polypeptide) in a baseline. According to a preferred embodiment, an increase or decrease of a polypeptide activity is quantified using a specific assay for the polypeptide activity.

Preferably, an increase of the expression level of a gene means an increase of at least 5% of the expression level of said gene using arrays. More preferably, an increase of the expression level of a gene means an increase of at least 10%, even more preferably at least 20%, at least 30%, at least 40%, at least 50%, at least 70%, at least 90%, at least 150% or more.

Preferably, a decrease of the expression level of a gene means a decrease of at least 5% of the expression level of said gene using arrays. More preferably, a decrease of the expression level of a gene means an decrease of at least 10%, even more preferably at least 20%., at least 30%, at least 40%, at least 50%, at least 70%, at least 90%, at least 150% or more.

Preferably, an increase of the expression level of a polypeptide means an increase of at least 5% of the expression level of said polypeptide using western blotting. More preferably, an increase of the expression level of a polypeptide means an increase of at least 10%, even more preferably at least 20%, at least 30%, at least 40%, at least 50%, at least 70%, at least 90%, at least 150% or more.

Preferably, a decrease of the expression level of a polypeptide means a decrease of at least 5% of the expression level of said polypeptide using western blotting. More preferably, a decrease of the expression level of a polypeptide means a decrease of at least 10%, even more preferably at least 20%, at least 30%, at least 40%, at least 50%, at least 70%, at least 90%, at least 150% or more.

Preferably, an increase of a polypeptide activity means an increase of at least 5% of said polypeptide activity using a suitable assay. More preferably, an increase of said polypeptide activity means an increase of at least 10%, even more preferably at least 20%, at least 30%, at least 40%, at least 50%, at least 70%, at least 90%, at least 150% or more.

Preferably, a decrease of a polypeptide activity means a decrease of at least 5% of said polypeptide activity using a suitable assay. More preferably, a decrease of said polypeptide activity means a decrease of at least 10%, even more preferably at least 20%, at least 30%, at least 40%, at least 50%, at least 70%, at least 90%, at least 150% or more.

In a preferred prognosticating method of the invention, the expression level of more than one, more preferably of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 35, 40, 45, 50, 55, 60, 65, 70, 75, 80, 85, 90, 95, 100, 105, 110, 115, 120, 125, 130, 135, 140 145, 150, 155, 160, 165, 168, 169 genes as defined herein, and/or the steady state levels of said corresponding polypeptides are determined. In another preferred method, a gene whose expression level is determined is selected in a combination of genes selected from the groups consisting of genes represented by the following sequences SEQ ID NO:1-32 or SEQ ID NO:1-169. Each combination of 1 till 32 genes of the first group, respectively 1 till 169 of the second group may be used. In a preferred embodiment, the 169 genes of the group SEQ ID NO:1-169 are being used. In another preferred embodiment, the 32 genes of the group SEQ ID NO:1-32 are being used. In another preferred embodiment, a gene from each cluster from the group formed by SEQ ID NO:1-32 is chosen. The genes classified as encoding enzymes are preferred. The gene FOSL1 is a preferred one. The gene ADORA2B is another preferred one. Table 3 identifies the 32 genes of SEQ ID NO:1-32 (annotation and accession numbers). The gene identified as number 1 will have its cDNA sequence being represented by SEQ ID NO:1. The same holds for other genes identified in Table 3. All the 169 genes represented by SEQ ID NO: 1-169 are identified in Table 2. Table 5 identifies the classification of genes into cluster and identifies their corresponding SEQ ID NO. The expression level of each of the 32 genes having SEQ ID NO 1-32 has been found to be up-regulated or increased in a metastasized cell by comparison to a non-metastasized cell. The genes presented in table 6 are also preferred. Table 6 identifies twelve Fra-1 regulated genes that were found to be essential for metastasis.

A reliable method for prognosticating metastasis may be carried out based on a sub combination of SEQ ID NO: 1-32 or of SEQ ID NO:1-169.

(Micro)arrays (or other high throughput screening devices) comprising the genes (nucleotides, nucleic acids), or polypeptides is a preferred way for carrying out a method of the invention. A microarray is a solid support or carrier containing one or more immobilised nucleic acid or polypeptide fragments for analysing nucleic acid or amino acid sequences or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001, Curr. Opin. Chem. Biol. 5: 40-45). (Micro)array technology allows for the measurement of the steady-state mRNA level of thousands of genes simultaneously thereby presenting a powerful tool for identifying gene modulation for a given group of genes as identified herein. Two microarray technologies are currently in wide use. The first are cDNA arrays and the second are oligonucleotide arrays. Although differences exist in the construction of these chips, essentially all downstream data analysis and output are the same. The product of these analyses are typically measurements of the intensity of the signal received from a labelled probe used to detect a cDNA sequence from the sample that hybridizes to a nucleic acid sequence at a known location on the microarray. Typically, the intensity of the signal is proportional to the quantity of cDNA, and thus mRNA, expressed in a cell from a cancer patient to be tested. A large number of such techniques are available and useful. Preferred methods for determining gene expression can be found in U.S. Pat. Nos. 6,271,002 to Linsley, et al.; 6,218,122 to Friend, et al.; 6,218,114 to Peck, et al.; and 6,004,755 to Wang, et al., the disclosure of each of which is incorporated herein by reference.

Analysis of the expression levels is conducted preferably by measuring expression levels using these techniques. Currently, this is best done by generating a matrix of the expression intensities of genes in a test sample (RNA from cells from a cancer patient to be tested) using a single channel hybridisation on a microarray platform, and comparing these intensities with the one of a reference group or baseline (in this case, a good and a poor prognosis centroid as earlier identified herein). For instance, the gene expression intensities from a non normal tissue (cancer) can be compared with the expression intensities generated from non normal tissues of the same type. It preferably means that within the context of the invention, a “control” refers to a large number of cancer patients as defined earlier herein preferably using the method as earlier defined herein.

Preferably, using its gene expression intensities, each sample is assigned to a good prognosis or bad prognosis group using a Single Sample Predictor. In this preferred method, each patient is assigned to the nearest centroid as determined by the highest Spearman rank order correlation score between the gene expression value of the corresponding gene sets of each sample and the centroid values of the ‘poor prognosis’ and ‘good prognosis’ centroid. A classifier of the invention is preferably used as described in Hu et al 2006.

In a further aspect, there is provided a second ex vivo method, wherein the method identified above is used to prognosticate the absence of metastasis in a cancer patient comprising identifying a lack of differential modulation of a gene (relative to the expression of a same gene in a control) in a combination of a gene selected from the groups consisting of genes represented by the following sequences SEQ ID NO:1-32 or SEQ ID NO:1-169.

All elements (for example type of cancer, identity of a patient, way of identifying a modulation of a gene) of said second method have already been identified for the first method. An absence of metastasis is preferably assessed the same way as earlier defined herein (scintigraphy or in an in vivo animal model). In a more preferred method, the absence of metastasis is prognosticated for a one, two, three, four, five year period or longer. Each of these methods may be optionally used for deciding a preferred treatment for the patient. For example, a patient for whom the gene expression pattern indicates a good prognosis (i.e. no metastasis) will receive standard treatment (i.e. less aggressive treatment). As another example, a patient for whom the gene expression pattern indicates a poor prognosis (i.e. metastasis) will receive a more aggressive treatment. Furthermore, the Fra-1 gene expression profile as identified herein (i.e. a combination of genes selected from the groups consisting of genes represented by the following sequences SEQ ID NO:1-32 or SEQ ID NO:1-169) may be used to identify those patients that are expected to benefit from a targeted inhibition of one or more genes from SEQ ID NO:1-32. In one preferred embodiment, one first identifies whether in a patient, preferably belonging to a poor prognosis group, there is a gene selected from the group SEQ ID NO:1-32 which is up regulated by comparison to a baseline as defined herein.

If such a gene is found, it is preferred to treat such an individual by using an inhibitor of said gene. The invention therefore allows a personalized treatment of this type of patient.

### Diagnostic Portfolio

Another aspect of the invention relates to a diagnostic portfolio comprising or consisting of isolated nucleic acid (or nucleotide) sequences, their complements, or portions thereof of a combination of genes selected from the groups consisting of a gene represented by the following sequences SEQ ID NO:1-32 or SEQ ID NO:1-169. Diagnostic portfolios comprising or consisting of any combinations or sub combinations as defined herein are also encompassed by the present invention.

A preferred diagnostic portfolio comprises a matrix suitable for identifying the differential expression of the genes contained therein. A more preferred diagnostic portfolio comprises a matrix, wherein said matrix is employed in a microarray. Said microarray is preferably a cDNA or oligonucleotide microarray.

Markers (i.e. genes or nucleic acids, nucleotides) used in a diagnostic portfolio have already been defined in the previous section.

### Kit

In a further aspect, there is provided an article including a representation of the gene expression profiles that make up the portfolios useful for prognosticating metastasis or prognosticating an absence of metastasis. These representations are reduced to a medium that can be automatically read by a machine such as computer readable media (magnetic, optical, and the like). The articles can also include instructions for assessing the gene expression profiles in such media. For example, the articles may comprise a CD ROM having computer instructions for comparing gene expression profiles of the portfolios of genes described above. The articles may also have gene expression profiles digitally recorded therein so that they may be compared with gene expression data from a cancer patient sample. Alternatively, the profiles can be recorded in different representational format. A graphical recordation is one such format. Different types of articles of manufacture according to the invention are media or formatted assays used to reveal gene expression profiles. These can comprise or consist of, for example, microarrays in which sequence complements or probes are affixed to a matrix to which the sequences indicative of the genes of interest combine creating a readable determinant of their presence. When such a microarray contains an optimized portfolio great savings in time, process steps, and resources are attained by minimizing the number of cDNA or oligonucleotides that must be applied to the substrate, reacted with the sample, read by an analyser, processed for results, and (sometimes) verified. Other articles according to the invention can be fashioned into reagent kits for conducting hybridization, amplification, and signal generation indicative of the level of expression of the genes in the portfolios as defined herein. Kits made according to the invention include formatted assays for determining the gene expression profiles. These can include all or some of the materials needed to conduct the assays such as reagents and instructions. Therefore, in a further aspect, there is provided a kit for prognosticating metastasis or prognosticating the absence of metastasis in a cancer patient comprising reagents for detecting nucleic acid sequences, their complements, or portions thereof in a combination of genes selected from the groups consisting of genes represented by the following sequences SEQ ID NO:1-32 or SEQ ID NO:1-169. Kits comprising or consisting of any combinations or sub combinations as defined herein are also encompassed by the present invention.

A preferred kit further comprises reagents for conducting a microarray analysis. More preferably, a kit further comprising a medium through which said nucleic acid sequences, their complements, or portions thereof are assayed. More preferably, said medium is a microarray. A kit may further comprise instructions.

### Inhibitor

In a further aspect, there is provided an inhibitor of a polypeptide, said polypeptide comprising an amino acid sequence that is encoded by a nucleotide sequence is selected from the groups consisting of:

(1) a nucleotide sequence encoding an enzyme ABHD11, AURKB, CHML, EZH2, FEN1, IGFBP3, PAICS, PCOLN3, PPP2R3A, PTGES, PTP4A1, and SCD,

(2) a nucleotide sequence encoding a transcription factor E2F1, FOSL1, and FOXM1,

(3) a nucleotide sequence encoding a structural protein C22orf18, CHAF1A, H2AFZ, SMTN, TJAP1, D21S2056E,

(4) a nucleotide sequence encoding a receptor ADORA2B;

(5) a nucleotide sequence encoding an adhesion molecule MTDH,

(6) a nucleotide sequence encoding an apoptose inhibitor BIRC5 and PHLDA1

(7) a nucleotide sequence encoding a protein involved in DNA replication/transcription MCM10, MCM2 and TRFP and

(8) a nucleotide sequence encoding a SEC14L1, SFN, SH3GL1 and YTHDF1,

said inhibitor being preferably for use as a medicament, more preferably for preventing, delaying and/or treating metastasis in a cancer patient.

This polypeptide may also be identified by referring to the nucleotide encoding it which is selected from the groups consisting of:

(1) a nucleotide sequence encoding an enzyme ABHD11, AURKB, CHML, EZH2, FEN1, IGFBP3, PAICS, PCOLN3, PPP2R3A, PTGES, PTP4A1, and SCD and that has at least 60% identity with SEQ ID NO:1, 3, 7, 10, 11, 15, 19, 20, 22, 23, 24, 25 and a nucleotide sequence that encodes an amino acid sequence that has at least 60% amino acid identity with an amino acid sequence encoded by a nucleotide sequence selected from SEQ ID NO: 1, 3, 7, 10, 11, 15, 19, 20, 22, 23, 24, 25,

(2) a nucleotide sequence encoding a transcription factor E2F1, FOSL1, and FOXM1 and that has at least 60% identity with SEQ ID NO: 9, 12, 13 and a nucleotide sequence that encodes an amino acid sequence that has at least 60% amino acid identity with an amino acid sequence encoded by a nucleotide sequence selected from SEQ ID NO: 9, 12, 13,

(3) a nucleotide sequence encoding a structural protein C22orf18, CHAF1A, H2AFZ, SMTN, TJAP1, D21S2056E and that has at least 60% identity with SEQ ID NO:5, 6, 14, 29, 30, 8 and a nucleotide sequence that encodes an amino acid sequence that has at least 60% amino acid identity with an amino acid sequence encoded by a nucleotide sequence selected from SEQ ID NO: 5, 6, 14, 29, 30, 8,

(4) a nucleotide sequence encoding a receptor ADORA2B and that has at least 60% identity with SEQ ID NO:2 and a nucleotide sequence that encodes an amino acid sequence that has at least 60% amino acid identity with an amino acid sequence encoded by a nucleotide sequence selected from SEQ ID NO:2,

(5) a nucleotide sequence encoding an adhesion molecule MTDH and that has at least 60% identity with SEQ ID NO:18 and a nucleotide sequence that encodes an amino acid sequence that has at least 60% amino acid identity with an amino acid sequence encoded by a nucleotide sequence selected from SEQ ID NO: 18,

(6) a nucleotide sequence encoding an apoptose inhibitor BIRC5 and PHLDA1 and that has at least 60% identity with SEQ ID NO:4, 21 and a nucleotide sequence that encodes an amino acid sequence that has at least 60% amino acid identity with an amino acid sequence encoded by a nucleotide sequence selected from SEQ ID NO:4, 21,

(7) a nucleotide sequence encoding a protein involved in DNA replication/transcription MCM10, MCM2 and TRFP and that has at least 60% identity with SEQ ID NO:16, 17, 31 and a nucleotide sequence that encodes an amino acid sequence that has at least 60% amino acid identity with an amino acid sequence encoded by a nucleotide sequence selected from SEQ ID NO: 16, 17, 31,

and

(8) a nucleotide sequence encoding a SEC14L1, SFN, SH3GL1 and YTHDF1 and that has at least 60% identity with SEQ ID NO: 26, 27, 28, 32 and a nucleotide sequence that encodes an amino acid sequence that has at least 60% amino acid identity with an amino acid sequence encoded by a nucleotide sequence selected from SEQ ID NO: 26, 27, 28, 32,

said inhibitor being preferably for use as a medicament, more preferably for preventing, delaying and/or treating metastasis in a cancer patient.

Inhibitors of enzymes as identified herein are preferred Inhibitors of FOSL1 are also preferred. Inhibitors of ADORA2B are also preferred.

An inhibitor of a polypeptide may also be defined as being an inhibitor of a polypeptide, said polypeptide comprising an amino acid sequence that is encoded by a nucleotide sequence is selected from the groups consisting of:

(a) a nucleotide sequence that has at least 60% identity with a sequence selected from SEQ ID NO: 1-32; and,
(b) a nucleotide sequence that encodes an amino acid sequence that has at least 60% amino acid identity with an amino acid sequence encoded by a nucleotide sequence selected from SEQ ID NO; 1-32,

Wherein said inhibitor is being preferably for use as a medicament, more preferably for preventing, delaying and/or treating metastasis in a cancer patient.

Through out the application, a polypeptide may be replaced by “a polypeptide comprising an amino acid sequence that is encoded by a nucleotide sequence selected from:

(a) a nucleotide sequence that has at least 60% identity with a sequence selected from SEQ ID NO: 1-32; and,
(b) a nucleotide sequence that encodes an amino acid sequence that has at least 60% amino acid identity with an amino acid sequence encoded by a nucleotide sequence selected from SEQ ID NO; 1-32” unless otherwise indicated.

An inhibitor is a compound which is able to decrease an activity of a polypeptide and/or to decrease its expression level and/or sub cellular localisation.

A “decrease of an activity of a polypeptide or a decrease of the expression level of gene or nucleotide encoding said polypeptide” is herein understood to mean any detectable change in a biological activity exerted by said polypeptide or in the expression level of said polypeptide as compared to said activity or expression of a wild type polypeptide such as the one encoded by SEQ ID NO:1-32. The decrease of the level or of the amount of a nucleotide encoding said polypeptide is preferably assessed using classical molecular biology techniques such as (real time) PCR, arrays or Northern analysis. Alternatively, according to another preferred embodiment, the decrease of the expression level of said polypeptide is determined directly by quantifying the amount of said polypeptide. Quantifying a polypeptide amount may be carried out by any known technique such as Western blotting or immunoassay using an antibody raised against said polypeptide. The skilled person will understand that alternatively or in combination with the quantification of a nucleic acid sequence and/or the corresponding polypeptide, a quantification of a substrate or a quantification of the expression of a target gene of said polypeptide or of any compound known to be associated with a function or activity of said polypeptide or the quantification of said function or activity of said polypeptide using a specific assay may be used to assess the decrease of an activity or expression level of said polypeptide.

Preferably, a decrease or a down-regulation of the expression level of a nucleotide sequence encoding said polypeptide means a decrease of at least 5% of the expression level of a nucleotide sequence using arrays or Northern blot. More preferably, a decrease of the expression level of a nucleotide sequence means an decrease of at least 10%, even more preferably at least 20%, at least 30%, at least 40%, at least 50%, at least 70%, at least 90%, at least 100%, or more. Preferably, the expression is no longer detectable. In another preferred embodiment, a decrease of the expression level of said polypeptide means a decrease of at least 5% of the expression level of said polypeptide using western blotting and/or using ELISA or a suitable assay. More preferably, a decrease of the expression level of said polypeptide means a decrease of at least 10%, even more preferably at least 20%, at least 30%, at least 40%, at least 50%, at least 70%, at least 90%, at least 150% or more. Preferably, the expression is no longer detectable. In another preferred embodiment, a decrease of a polypeptide activity means a decrease of at least 5% of said activity using a suitable assay as earlier defined herein. More preferably, a decrease of said activity means a decrease of at least 10%, even more preferably at least 20%, at least 30%, at least 40%, at least 50%, at least 70%, at least 90%, at least 150% or more. Preferably, said activity is no longer detectable

An inhibitor may be any compound. The invention also provides a method for identifying additional inhibitors of a polypeptide (see later herein). Preferably an inhibitor is a DNA or RNA molecule, a dominant negative molecule, an inhibiting antibody raised against said polypeptide, a peptide-like molecule (referred to as peptidomimetics) or a non-peptide molecule. Each of these inhibitors is presented in more details below. An inhibitor may act at the level of the polypeptide itself, e.g. by providing an antagonist or inhibitor of said polypeptide to a cell, such as e.g. an inhibiting antibody raised against said polypeptide (named an antibody herein) or a dominant negative form of said polypeptide or an antisense (named antisense molecule herein). An antibody, an antisense molecule or a dominant negative of the invention may be obtained as described below. Alternatively, an inhibitor may act at the level of the nucleotide encoding said polypeptide. In this case, the expression level of polypeptide is decreased by regulating the expression level of a nucleotide sequence encoding said polypeptide.

Accordingly in a first preferred embodiment, an inhibitor is a DNA molecule.

The invention provides first a nucleic acid construct comprising all or a part of a nucleotide sequence that encodes a polypeptide that comprises an amino acid sequence that is encoded by a nucleotide sequence selected from:

(a) a nucleotide sequence that has at least 60, 70, 80, 85, 90, 95, 98 or 99% identity with a nucleotide sequence selected from SEQ ID NO:1-32; and/or,

(b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO:1-32.

Preferably, a nucleotide sequence is operably linked to a promoter that is capable of driving expression of said nucleotide sequence in a cell, more preferably a human and/or tumour cell. Even more preferably, the cell is a human breast cell.

Accordingly, in a more preferred embodiment, a nucleic acid construct of the invention comprises or consists of a nucleotide sequence that encodes an RNAi agent, i.e. an RNA molecule that is capable of RNA interference or that is part of an RNA molecule that is capable of RNA interference. Such RNA molecules are referred to as small RNA molecules such as siRNA (short interfering RNA, including e.g. a short hairpin RNA). The nucleotide sequence that encodes the RNAi agent preferably has sufficient complementarity with a cellular nucleotide sequence to be capable of inhibiting the expression of a polypeptide that comprises an amino acid sequence that is encoded by a nucleotide sequence selected from:

a) a nucleotide sequence that has at least 60, 70, 80, 85, 90, 95, 98 or 99% identity with a sequence selected from SEQ ID NO: 1-32; and/or,

(b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO: 1-32;

In an even more preferred embodiment, a nucleic acid construct of the invention comprises or consists of a nucleotide sequence that encodes an RNAi agent capable of inhibiting the expression of a polypeptide that comprises an amino acid sequence that is encoded by a nucleotide sequence selected from:

a) a nucleotide sequence that has at least 60, 70, 80, 85, 90, 95, 98 or 99% identity with SEQ ID NO:1, 2, 3, 7, 10, 11, 15, 19, 20, 22, 23, 24, 25, 12 as defined herein; and/or,

(b) a nucleotide sequence that encodes an amino acid sequence that has at least 60, 70, 80, 85, 90, 95, 98 or 99% amino acid identity with an amino acid sequence encoded by a nucleotide sequence selected from SEQ ID NO: 1, 2, 3, 7, 10, 11, 15, 19, 20, 22, 23, 24, 25, 12;

wherein optionally the nucleotide sequence encoding the RNAi agent is operably linked to a promoter that is capable of driving expression of the nucleotide sequence in a cell, more preferably a human and/or tumour cell. Even more preferably, the cell is a human breast cell.

The role of each of these genes in metastasis has been unambiguously demonstrated in the example of this application. Therefore, any substance including a nucleic acid construct comprising a sequence encoding an RNAi agent capable of down regulating the expression level of any one of these genes or of any combination thereof as defined herein is a preferred embodiment according to the invention. However, any other substance having this capacity of down regulating the expression level of any of the genes identified by SEQ ID NO:1-32 and preferably identified in a method of the invention as later defined herein is encompassed by the present invention.

Alternatively or in combination with the antisense approach, one may also use an inactivating approach. In this approach, an inactivating nucleic acid construct is introduced into a cell. Said inactivating construct comprises or consists of a nucleotide molecule which is designed in order to inactivate the expression of a polypeptide. The skilled person knows how to design an inactivating construct. For example, at least part of a gene encoding a polypeptide is replaced by a marker such as the neomycine gene.

Alternatively or in combination with the antisense and inactivating approaches, one may also use a dominant negative approach. In this approach, a nucleic acid construct is introduced into a cell, wherein said nucleic construct comprises a dominant negative nucleotide sequence that is capable of inhibiting or down-regulating an activity of a corresponding endogenous polypeptide, and wherein, optionally, a dominant negative nucleotide sequence is under the control of a promoter capable of driving expression of said dominant negative nucleotide sequence in a cell. In a preferred embodiment described earlier herein, a nucleic acid construct used herein comprises or consists of a dominant negative of a polypeptide as earlier defined herein. Alternatively, a dominant negative molecule may be directly administered to a subject. The skilled person knows how to design a dominant negative of a polypeptide. Several strategies are already known for designing a dominant negative of a polypeptide depending on the function or activity of said polypeptide. If a polypeptide is a kinase, a dominant negative kinase is usually a truncated kinase without a catalytic domain(s) or with an inactive catalytic domain(s). An inactive catalytic domain may be generated by introducing a point-mutation(s) in said kinase domain(s).

In a nucleic acid construct of the invention (a dominant negative approach, inactivating approach or antisense approach), a promoter which may be present is preferably a promoter that is specific for a human and/or tumour cell and/or mammary cell. More preferably, a promoter chosen is specific for and functional in a human and/or tumour cell and/or mammary cell. A promoter that is specific for a human and/or tumour cell and/or mammary is a promoter with a transcription rate that is higher in such a cell than in other types of cells. Preferably the promoter's transcription rate in such a cell is at least 1.1, 1.5, 2.0 or 5.0 times higher than in a other types of cells as measured by PCR of the construct in such a cell as compared to other types of cells.

A nucleic acid construct as defined herein is for use as a medicament, preferably for preventing, delaying and/or treating metastasis in a cancer patient.

In a preferred embodiment a nucleic acid construct is a viral gene therapy vector selected from gene therapy vectors based on an adenovirus, an adeno-associated virus (AAV), a herpes virus, a pox virus and a retrovirus. A preferred viral gene therapy vector is an AAV or Lentiviral vector. Such vectors are further described herein below.

In addition, for some of the polypeptides as defined herein inhibitors have already been identified (see table 4). In addition, inhibitors of ADORA2B are also known: 7-Chloro-4-hydroxy-2-phenyl-1,8-naphthyridine, an A1 adenosine receptor antagonist, CGS-15943, a Highly potent, non-selective A1 adenosine receptor antagonist. These two compounds are commercially available (Sigma). Therefore the invention also encompasses each of these inhibitors for use as a medicament, preferably for preventing, delaying and/or treating metastasis in a cancer patient.

### Use of a Nucleic Acid Construct

In a further aspect the invention relates to a use of a nucleic acid construct as defined herein for modulating the expression level of a gene and/or activity or steady state level of a polypeptide as defined herein, for the manufacture of a medicament for preventing and/or delaying and/or metastasis in a cancer patient, preferably in a method of the invention as defined herein.

Identification of a Substance Able to Prevent, Delay and/or Treat Metastasis in a Cancer Patient

In yet a further aspect, the invention relates to a method for identification of a substance capable of preventing, delaying and/or treating metastasis in a cancer patient.

The method preferably comprises the steps of:

(a) providing a test cell population capable of expressing a nucleotide sequence as present in a nucleic acid construct, wherein said nucleotide sequence is a nucleotide sequence that has at least 60% identity with a sequence selected from SEQ ID NO: 1-32 as identified in claim 1 or SEQ ID NO:1-169 and,
a nucleotide sequence that encodes an amino acid sequence that has at least 60% amino acid identity with an amino acid sequence encoded by a nucleotide sequence selected from SEQ ID NO; 1-32 or SEQ ID NO:1-169;
(b) contacting the test cell population with the substance;
(c) determining the expression level of the nucleotide sequence or the activity or steady state level of the polypeptide in the test cell population contacted with the substance;
(d) comparing the expression, activity or steady state level determined in (c) with the expression, activity or steady state level of the nucleotide sequence or of the polypeptide in a test cell population that has not been contacted with the substance; and, (e) identifying a substance that produces a difference in expression level, activity or steady state level of the nucleotide sequence or the polypeptide, between the test cell population that has been contacted with the substance and the test cell population that has not been contacted with the substance.

Preferably, in step a), the test cell comprises a nucleic acid construct of the invention. Preferably, in a method the expression levels, activities or steady state levels of more than one nucleotide sequence or more than one polypeptide are compared. Preferably, in a method, a test cell population comprises mammalian cells, more preferably human and/or tumour cells. Even more preferably, a test cell population comprises bone-marrow and/or peripheral blood and/or pluripotent stem cells and/or mammary cells. These cells can be harvested, purified using techniques known to the skilled person. Even more preferably, a test cell population comprises a cell line. Preferably the cell line is a human or rat cell line. Even more preferably, the human cell line LM2 or the rat cell line RK3E is being used. In another preferred embodiment, test cells are part of an in vivo animal model as earlier defined herein. In one aspect the invention also pertains to a substance that is identified in a method the aforementioned methods.

In a preferred embodiment, “preventing” metastasis means that during at least one, two, three, four, five years, or longer no metastatic lesion will be detected in an in vivo animal model as earlier defined herein and/or in a cancer patient using scintigraphy as earlier defined herein, wherein said tumour cells were treated with said substance by comparison with the potential development of a metastatic lesion in a non-treated control.

In a preferred embodiment, “delaying” metastasis means that the detection of a metastatic lesion in a given system using the same assays as defined in the previous paragraph treated with said substance is delayed of at least 1, 6, 12, 18, 24, 30, 36, 42, 48, 54, 60, 66 months or longer compared to the time at which detection of one metastatic lesion will occur in a corresponding control non treated with said substance.

In a preferred embodiment, “treating” metastasis means that there is a detectable decrease of the amount of metastatic lesions in a given system using the same assays as defined in the previous paragraph treated with said substance after at least one month (2, 3, 4, 5, 6, 7, 8, 9, 10, 11, 12 months or longer) compared to the amount of metastatic lesions in the same system which has not been treated. A detectable decrease is preferably defined as being at least 1% decrease, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more till no metastase are detectable.

Method for Preventing, Delaying and/or Treating Metastasis

There is currently no known medicament that may be used in such a method in a cancer patient. The only standard treatments comprise irradiation, hormonal therapy and/or chemotherapy. Accordingly, in a further aspect, the invention provides a method for preventing, delaying and/or treating metastasis in a cancer patient, said method comprising pharmacologically altering the expression level of a gene and/or activity or the steady-state level of a polypeptide encoded by a nucleotide sequence selected from the genes or nucleotide sequences identified in the section entitled “inhibitor”. In this section a polypeptide means a polypeptide for which encoding sequence has been identified in the section entitled “inhibitor”. In a preferred method of the invention, the expression level of a gene and/or activity and/or steady-state level of said polypeptide of is altered in order to mimic its physiological level in a cancer patient known not have metastasis (no detectable metastase) or in a healthy subject.

The expression “preventing, delaying and/or treating metastasis” is given the same meaning as in previous section.

The activity or steady-state level of a polypeptide may be altered at the level of the polypeptide itself, e.g. by providing a antagonist or inhibitor of a polypeptide to a patient, preferably to a cell, more preferably to a tumour cell of said cancer patient such as e.g. an antibody against a polypeptide, preferably a neutralizing antibody. For provision of a dominant negative polypeptide or antisense from an exogenous source, a dominant negative polypeptide or antisense may conveniently be produced by expression of a nucleic acid encoding a dominant negative polypeptide or antisense in a suitable host cell as described below. An antibody against a polypeptide of the invention may be obtained as described below.

Preferably, however, the activity or steady-state level of a polypeptide is altered by regulating the expression level of a nucleotide sequence encoding a polypeptide. Preferably, the expression level of a nucleotide sequence is regulated in a human and/or tumour cell.

The expression level of a polypeptide may be decreased by providing an inhibitor, preferably an antisense molecule to a human and/or tumour cell, whereby an antisense molecule is capable of inhibiting the biosynthesis (usually the translation) of a nucleotide sequence encoding a polypeptide. Decreasing gene expression by providing antisense or interfering RNA molecules is described below herein and is e.g. reviewed by Famulok et al. (2002, Trends Biotechnol., 20(11): 462-466). An antisense molecule may be provided to a cell as such or it may be provided by introducing an expression construct into a human and/or tumour cell, whereby an expression construct comprises an antisense nucleotide sequence that is capable of inhibiting the expression of a nucleotide sequence encoding a polypeptide, and whereby an antisense nucleotide sequence is under control of a promoter capable of driving transcription of an antisense nucleotide sequence in a human and/or tumour cell. The expression level of a polypeptide may also be decreased by introducing an expression construct into a human and/or tumour cell, whereby an expression construct comprises a nucleotide sequence encoding a factor capable of trans-repression of an endogenous nucleotide sequence encoding a polypeptide. An antisense or interfering nucleic acid molecule may be introduced into a cell directly “as such”, optionally in a suitable formulation, or it may be produce in situ in a cell by introducing into a cell an expression construct comprising a (antisense or interfering) nucleotide sequence that is capable of inhibiting the expression of a nucleotide sequence encoding a polypeptide, whereby, optionally, an antisense or interfering nucleotide sequence is under control of a promoter capable of driving expression of an nucleotide sequence in a human and/or tumour cell.

The meaning of “increase or decrease the expression of a gene (nucleotide) or corresponding polypeptide” is the same as given in the section entitled “ex vivo methods”.

A method of the invention preferably comprises the step of administering to a cancer patient a therapeutically effective amount of a pharmaceutical composition comprising an inhibitor as defined herein: a nucleic acid construct for modulating the activity or steady state level of a polypeptide and/or a neutralizing antibody and/or a polypeptide as defined herein. A nucleic acid construct may be an expression construct as further specified herein below. Preferably, an expression construct is a viral gene therapy vector selected from a gene therapy vector based on an adenovirus, an adeno-associated virus (AAV), a herpes virus, a pox virus and a retrovirus. A preferred viral gene therapy vector is an AAV or Lentiviral vector. Alternatively, a nucleic acid construct may be for inhibiting expression of a polypeptide of the invention such as an antisense molecule or an RNA molecule capable of RNA interference (see below). In a method of the invention, a human and/or tumour cell is preferably a cell from a cancer patient suspected to have a high risk of having a metastasised cancer, due for example to its age and/or its genetic background and/or to its diet and/or to the type of cancer he has. Alternatively, in another preferred embodiment, a method of the invention is applied on a cell from a cancer patient diagnosed as having a risk of having a metastasised cancer. A prognosticating method used is preferably one of the inventions already earlier described herein. More preferably, if in such method it has been found that in a patient preferably belonging to a poor prognosis group there is a gene selected from the group SEQ ID NO:1-32 whose expression is up regulated by comparison to a baseline as defined herein, it is preferred to treat such an individual or patient by using an inhibitor of said gene. The invention therefore allows a personalized treatment of this type of patient.

In a method, a human and/or tumour cell chosen to be treated are preferably isolated from the patient they belong to (ex vivo method). Cells are subsequently treated by altering the activity or the steady state level of a polypeptide of the invention. This treatment is preferably performed by infecting them with a polypeptide and/or a nucleic acid construct of the invention and/or a neutralizing antibody as earlier defined herein. Finally, treated cells are placed back into the patient they belong to.

In another treating method, the invention mentioned herein may be combined with standard treatments of metastasis such as chemotherapy and/or radiation.

Although gene therapy is a possibility for preventing, delaying and/or treating metastasis, other possible treatments may also be envisaged. For example, treatment by “small molecule” drugs to steer certain molecular pathways in the desired direction, is also preferred. These small molecules are preferably identified by the screening method of the invention as defined later herein.

### Genes Defined by a SEQ ID NO and Sequence Identity

It is to be understood that each gene as identified herein by a given Sequence Identity Number (SEQ ID NO 1-169) is not limited to this specific sequence as disclosed. Each gene sequence or nucleotide sequence as identified herein encodes a given protein or polypeptide as identified in Table 3. Throughout this application, each time one refers to a specific nucleotide sequence SEQ ID NO (take SEQ ID NO:1 as example), one may replace it by:

i. a polypeptide comprising an amino acid sequence that has at least 60% sequence identity with an amino acid sequence SEQ ID NO:1 as identified in Table 3 or in the list of sequences provided herewith as being encoded by SEQ ID NO:1,

ii. a nucleotide sequence comprising a nucleotide sequence that has at least 60% sequence identity with SEQ ID NO:1 (as example).

iii. a nucleotide sequences the complementary strand of which hybridizes to a nucleic acid molecule of sequence of (i) or (ii);

iv. a nucleotide sequence the sequence of which differs from the sequence of a nucleic acid molecule of (iii) due to the degeneracy of the genetic code.

iv. a nucleotide sequence that encodes an amino acid sequence that has at least 60% amino acid identity with an amino acid sequence encoded by a nucleotide sequence SEQ ID NO:1.

Each nucleotide sequence or amino acid sequence described herein by virtue of its identity percentage (at least 60%) with a given nucleotide sequence or amino acid sequence respectively has in a further preferred embodiment an identity of at least 65%, 70%, 75%, 80%, 85%, 90%, 95%, 97%, 98%, 99% or more identity with the given nucleotide or amino acid sequence respectively. In a preferred embodiment, sequence identity is determined by comparing the whole length of the sequences as identified herein.

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

### Recombinant Techniques and Methods for Recombinant Production of a Polypeptide

If an inhibitor is a polypeptide, said polypeptide can be prepared using recombinant techniques, in which a nucleotide sequence encoding said polypeptide of interest is expressed in a suitable host cell. The present invention thus also concerns the use of a nucleic acid construct, preferably being a vector comprising a nucleic acid molecule being represented by a nucleotide sequence as defined above. Preferably the vector is a replicative vector comprising on origin of replication (or autonomously replication sequence) that ensures multiplication of the vector in a suitable host for the vector. Alternatively the vector is capable of integrating into a host cell's genome, e.g. through homologous recombination or otherwise. A particularly preferred vector is an expression vector wherein a nucleotide sequence encoding a polypeptide as defined above, is operably linked to a promoter capable of directing expression of the coding sequence in a host cell for the vector.

As used herein, the term “promoter” refers to a nucleic acid fragment that functions to control the transcription of one or more genes, located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is only active in specific types of differentiated cells/tissues, such as preferably a human and/or tumour and/or mammary cell or tissue derived thereof.

An expression vector may allow a polypeptide of the invention as defined above to be prepared using recombinant techniques in which a nucleotide sequence encoding said polypeptide is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Typically, a nucleic acid encoding said polypeptide is used in an expression vector. The phrase “expression vector” generally refers to nucleotide sequences that are capable of effecting expression of a gene in hosts compatible with such sequences. These expression vectors typically include at least suitable promoter sequences and optionally, transcription termination signals. Additional factors necessary or helpful in effecting expression can also be used as described herein. A nucleic acid or DNA encoding said polypeptide is incorporated into a DNA construct capable of introduction into and expression in an in vitro cell culture. Specifically, DNA constructs are suitable for replication in a prokaryotic host, such as bacteria, e.g., E. coli, or can be introduced into a cultured mammalian, plant, insect, e.g., Sf9, yeast, fungi or other eukaryotic cell lines.

DNA constructs prepared for introduction into a particular host typically include a replication system recognized by the host, the intended DNA segment encoding the desired polypeptide, and transcriptional and translational initiation and termination regulatory sequences operably linked to the polypeptide-encoding segment. A DNA segment is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a pre protein that participates in the secretion of said polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

The selection of an appropriate promoter sequence generally depends upon the host cell selected for the expression of the DNA segment. Examples of suitable promoter sequences include prokaryotic, and eukaryotic promoters well known in the art (see, e.g. Sambrook and Russell, 2001, supra). The transcriptional regulatory sequences typically include a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). Expression vectors include the replication system and transcriptional and translational regulatory sequences together with the insertion site for the polypeptide encoding segment can be employed. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. The host cells may thus be prokaryotic or eukarotic host cells. A host cell may be a host cell that is suitable for culture in liquid or on solid media. A host cell is preferably used in a method for producing a polypeptide of the invention as defined above or in a method for identification of a substance as defined herein. Said method may comprise the step of culturing a host cell under conditions conducive to the expression of said polypeptide. Optionally the method may comprise recovery of said polypeptide. A polypeptide may e.g. be recovered from the culture medium by standard protein purification techniques, including a variety of chromatography methods known in the art per se.

Alternatively, a host cell is a cell that is part of a multi cellular organism such as a transgenic plant or animal, preferably a non-human animal. A transgenic plant comprises in at least a part of its cells a vector as defined above. Methods for generating transgenic plants are e.g. described in U.S. Pat. No. 6,359,196 and in the references cited therein. Such transgenic plant or animal may be used in a method for producing a polypeptide of the invention as defined above and/or in a method for identification of a substance both as defined herein. For transgenic plant, a preferred method comprises the step of recovering a part of a transgenic plant comprising in its cells the vector or a part of a descendant of such transgenic plant, whereby the plant part contains said polypeptide, and, optionally recovery of said polypeptide from the plant part. Such methods are also described in U.S. Pat. No. 6,359,196 and in the references cited therein. Similarly, the transgenic animal comprises in its somatic and germ cells a vector as defined above. The transgenic animal preferably is a non-human animal. Methods for generating transgenic animals are e.g. described in WO 01/57079 and in the references cited therein. Such transgenic animals may be used in a method for producing a polypeptide of the invention as defined above, the method comprising the step of recovering a body fluid from a transgenic animal comprising the vector or a female descendant thereof, wherein the body fluid contains said polypeptide, and, optionally recovery of said polypeptide from said body fluid. Such methods are also described in WO 01/57079 and in the references cited therein. The body fluid containing the polypeptide preferably is blood or more preferably milk

Another method for preparing a polypeptide is to employ an in vitro transcription/translation system. DNA encoding a polypeptide is cloned into an expression vector as described supra. The expression vector is then transcribed and translated in vitro. The translation product can be used directly or first purified. A polypeptide resulting from in vitro translation typically do not contain the post-translation modifications present on polypeptides synthesised in vivo, although due to the inherent presence of microsomes some post-translational modification may occur. Methods for synthesis of polypeptides by in vitro translation are described by, for example, Berger & Kimmel, Methods in Enzymology, Volume 152, Guide to Molecular Cloning Techniques, Academic Press, Inc., San Diego, Calif., 1987.

### Gene Therapy

Some aspects of the invention concern the use of a nucleic acid construct or expression vector comprising a nucleotide sequence as defined above, wherein the vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Cum Opin. Biotechnol. 10: 454-7; Sommerfelt, 1999, J. Gen. Virol. 80: 3049-64; Reiser, 2000, Gene Ther. 7: 910-3; and references cited therein.

Particularly suitable gene therapy vectors include Adenoviral and Adeno-associated virus (AAV) vectors. These vectors infect a wide number of dividing and non-dividing cell types including neuronal cells. In addition adenoviral vectors are capable of high levels of transgene expression. However, because of the episomal nature of the adenoviral and AAV vectors after cell entry, these viral vectors are most suited for therapeutic applications requiring only transient expression of the transgene (Russell, 2000, J. Gen. Virol. 81: 2573-2604; Goncalves, 2005, Virol J. 2(1):43) as indicated above. Preferred adenoviral vectors are modified to reduce the host response as reviewed by Russell (2000, supra). Method for neuronal gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55. For gene transfer into a human and/or tumour and/or mammary cell, a AAV serotype 2 is an effective vector and therefore a preferred AAV serotype.

A preferred retroviral vector for application in the present invention is a lentiviral based expression construct. Lentiviral vectors have the unique ability to infect non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Generally, gene therapy vectors will be as the expression vectors described above in the sense that they comprise a nucleotide sequence encoding a polypeptide of the invention to be expressed, whereby said nucleotide sequence is operably linked to the appropriate regulatory sequences as indicated above. Such regulatory sequence will at least comprise a promoter sequence. Suitable promoters for expression of a nucleotide sequence encoding said polypeptide from gene therapy vectors include e.g. cytomegalovirus (CMV) intermediate early promoter, viral long terminal repeat promoters (LTRs), such as those from murine moloney leukaemia virus (MMLV) rous sarcoma virus, or HTLV-1, the simian virus 40 (SV 40) early promoter and the herpes simplex virus thymidine kinase promoter. Suitable promoters are described below.

Several inducible promoter systems have been described that may be induced by the administration of small organic or inorganic compounds. Such inducible promoters include those controlled by heavy metals, such as the metallothionine promoter (Brinster et al. 1982 Nature 296: 39-42; Mayo et al. 1982 Cell 29: 99-108), RU-486 (a progesterone antagonist) (Wang et al. 1994 Proc. Natl. Acad. Sci. USA 91: 8180-8184), steroids (Mader and White, 1993 Proc. Natl. Acad. Sci. USA 90: 5603-5607), tetracycline (Gossen and Bujard 1992 Proc. Natl. Acad. Sci. USA 89: 5547-5551; U.S. Pat. No. 5,464,758; Furth et al. 1994 Proc. Natl. Acad. Sci. USA 91: 9302-9306; Howe et al. 1995 J. Biol. Chem. 270: 14168-14174; Resnitzky et al. 1994 Mol. Cell. Biol. 14: 1669-1679; Shockett et al. 1995 Proc. Natl. Acad. Sci. USA 92: 6522-6526) and the tTAER system that is based on the multi-chimeric transactivator composed of a tetR polypeptide, as activation domain of VP16, and a ligand binding domain of an estrogen receptor (Yee et al., 2002, U.S. Pat. No. 6,432,705).

Suitable promoters for nucleotide sequences encoding small RNAs for knock down of specific genes by RNA interference (see below) include, in addition to the above mentioned polymerase II promoters, polymerase III promoters. The RNA polymerase III (pol III) is responsible for the synthesis of a large variety of small nuclear and cytoplasmic non-coding RNAs including 5S, U6, adenovirus VA1, Vault, telomerase RNA, and tRNAs. The promoter structures of a large number of genes encoding these RNAs have been determined and it has been found that RNA pol III promoters fall into three types of structures (for a review see Geiduschek and Tocchini-Valentini, 1988 Annu. Rev. Biochem. 57: 873-914; Willis, 1993 Eur. J. Biochem. 212: 1-11; Hernandez, 2001, J. Biol. Chem. 276: 26733-36). Particularly suitable for expression of siRNAs are the type 3 of the RNA pol III promoters, whereby transcription is driven by cis-acting elements found only in the 51-flanking region, i.e. upstream of the transcription start site. Upstream sequence elements include a traditional TATA box (Mattaj et al., 1988 Cell 55, 435-442), proximal sequence element and a distal sequence element (DSE; Gupta and Reddy, 1991 Nucleic Acids Res. 19, 2073-2075). Examples of genes under the control of the type 3 pol III promoter are U6 small nuclear RNA (U6 snRNA), 7SK, Y, MRP, H1 and telomerase RNA genes (see e.g. Myslinski et al., 2001, Nucl. Acids Res. 21: 2502-09).

The gene therapy vector may optionally comprise a second or one or more further nucleotide sequence coding for a second or further polypeptide. The second or further polypeptide may be a (selectable) marker polypeptide that allows for the identification, selection and/or screening for cells containing the expression construct. Suitable marker proteins for this purpose are e.g. the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

Alternatively, the second or further nucleotide sequence may encode a polypeptide that provides for fail-safe mechanism that allows to cure a subject from the transgenic cells, if deemed necessary. Such a nucleotide sequence, often referred to as a suicide gene, encodes a polypeptide that is capable of converting a pro drug into a toxic substance that is capable of killing the transgenic cells in which said polypeptide is expressed. Suitable examples of such suicide genes include e.g. the E. coli cytosine deaminase gene or one of the thymidine kinase genes from Herpes Simplex Virus, Cytomegalovirus and Varicella-Zoster virus, in which case ganciclovir may be used as prodrug to kill the IL-10 transgenic cells in the subject (see e.g. Clair et al., 1987, Antimicrob. Agents Chemother. 31: 844-849).

A gene therapy vector is preferably formulated in a pharmaceutical composition comprising a suitable pharmaceutical carrier as defined below.

### RNA Interference

For knock down of expression of a specific polypeptide of the invention as identified in the section entitled “inhibitor”, a gene therapy vector or other expression construct is used for the expression of a desired nucleotide sequence that preferably encodes an RNAi agent, i.e. an RNA molecule that is capable of RNA interference or that is part of an RNA molecule that is capable of RNA interference. Such RNA molecules are referred to as siRNA (short interfering RNA, including e.g. a short hairpin RNA). Alternatively, the siRNA molecules may directly, e.g. in a pharmaceutical composition that is administered within or in the neighbourhood of a human and/or tumour and/or mammary cell.

A desired nucleotide sequence comprises an antisense code DNA coding for the antisense RNA directed against a region of the target gene mRNA, and/or a sense code DNA coding for the sense RNA directed against the same region of the target gene mRNA. In a DNA construct of the invention, the antisense and sense code DNAs are operably linked to one or more promoters as herein defined above that are capable of expressing the antisense and sense RNAs, respectively. “siRNA” means a small interfering RNA that is a short-length double-stranded RNA that are not toxic in mammalian cells (Elbashir et al., 2001, Nature 411: 494-98; Caplen et al., 2001, Proc. Natl. Acad. Sci. USA 98: 9742-47). The length is not necessarily limited to 21 to 23 nucleotides. There is no particular limitation in the length of siRNA as long as it does not show toxicity. “siRNAs” can be, e.g. at least 15, 18 or 21 nucleotides and up to 25, 30, 35 or 49 nucleotides long. Alternatively, the double-stranded RNA portion of a final transcription product of siRNA to be expressed can be, e.g. at least 15, 18 or 21 nucleotides and up to 25, 30, 35 or 49 nucleotides long.

“Antisense RNA” is an RNA strand having a sequence complementary to a target gene mRNA, and thought to induce RNAi by binding to the target gene mRNA. “Sense RNA” has a sequence complementary to the antisense RNA, and annealed to its complementary antisense RNA to form siRNA. The term “target gene” in this context refers to a gene whose expression is to be silenced due to siRNA to be expressed by the present system, and can be arbitrarily selected. As this target gene, for example, genes whose sequences are known but whose functions remain to be elucidated, and genes whose expressions are thought to be causative of diseases are preferably selected. A target gene may be one whose genome sequence has not been fully elucidated, as long as a partial sequence of mRNA of the gene having at least 15 nucleotides or more, which is a length capable of binding to one of the strands (antisense RNA strand) of siRNA, has been determined. Therefore, genes, expressed sequence tags (ESTs) and portions of mRNA, of which some sequence (preferably at least 15 nucleotides) has been elucidated, may be selected as the “target gene” even if their full length sequences have not been determined.

The double-stranded RNA portions of siRNAs in which two RNA strands pair up are not limited to the completely paired ones, and may contain non pairing portions due to mismatch (the corresponding nucleotides are not complementary), bulge (lacking in the corresponding complementary nucleotide on one strand), and the like. Non pairing portions can be contained to the extent that they do not interfere with siRNA formation. The “bulge” used herein preferably comprise 1 to 2 non pairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. Such non pairing portions (mismatches or bulges, etc.) can suppress the below-described recombination between antisense and sense code DNAs and make the siRNA expression system as described below stable. Furthermore, although it is difficult to sequence stem loop DNA containing no non pairing portion in the double-stranded RNA region of siRNAs in which two RNA strands pair up, the sequencing is enabled by introducing mismatches or bulges as described above. Moreover, siRNAs containing mismatches or bulges in the pairing double-stranded RNA region have the advantage of being stable in E. coli or animal cells.

The terminal structure of siRNA may be either blunt or cohesive (overhanging) as long as siRNA enables to silence the target gene expression due to its RNAi effect. The cohesive (overhanging) end structure is not limited only to the 3′ overhang, and the 5′ overhanging structure may be included as long as it is capable of inducing the RNAi effect. In addition, the number of overhanging nucleotide is not limited to the already reported 2 or 3, but can be any numbers as long as the overhang is capable of inducing the RNAi effect. For example, the overhang consists of 1 to 8, preferably 2 to 4 nucleotides. Herein, the total length of siRNA having cohesive end structure is expressed as the sum of the length of the paired double-stranded portion and that of a pair comprising overhanging single-strands at both ends. For example, in the case of 19 by double-stranded RNA portion with 4 nucleotide overhangs at both ends, the total length is expressed as 23 bp. Furthermore, since this overhanging sequence has low specificity to a target gene, it is not necessarily complementary (antisense) or identical (sense) to the target gene sequence. Furthermore, as long as siRNA is able to maintain its gene silencing effect on the target gene, siRNA may contain a low molecular weight RNA (which may be a natural RNA molecule such as tRNA, rRNA or viral RNA, or an artificial RNA molecule), for example, in the overhanging portion at its one end.

In addition, the terminal structure of the “siRNA” is necessarily the cut off structure at both ends as described above, and may have a stem-loop structure in which ends of one side of double-stranded RNA are connected by a linker RNA (a “shRNA”). The length of the double-stranded RNA region (stem-loop portion) can be, e.g. at least 15, 18 or 21 nucleotides and up to 25, 30, 35 or 49 nucleotides long. Alternatively, the length of the double-stranded RNA region that is a final transcription product of siRNAs to be expressed is, e.g. at least 15, 18 or 21 nucleotides and up to 25, 30, 35 or 49 nucleotides long. Furthermore, there is no particular limitation in the length of the linker as long as it has a length so as not to hinder the pairing of the stem portion. For example, for stable pairing of the stem portion and suppression of the recombination between DNAs coding for the portion, the linker portion may have a clover-leaf tRNA structure. Even though the linker has a length that hinders pairing of the stem portion, it is possible, for example, to construct the linker portion to include introns so that the introns are excised during processing of precursor RNA into mature RNA, thereby allowing pairing of the stem portion. In the case of a stem-loop siRNA, either end (head or tail) of RNA with no loop structure may have a low molecular weight RNA. As described above, this low molecular weight RNA may be a natural RNA molecule such as tRNA, rRNA, snRNA or viral RNA, or an artificial RNA molecule.

To express antisense and sense RNAs from the antisense and sense code DNAs respectively, a DNA construct of the present invention comprise a promoter as defined above. The number and the location of the promoter in the construct can in principle be arbitrarily selected as long as it is capable of expressing antisense and sense code DNAs. As a simple example of a DNA construct of the invention, a tandem expression system can be formed, in which a promoter is located upstream of both antisense and sense code DNAs. This tandem expression system is capable of producing siRNAs having the aforementioned cut off structure on both ends. In the stem-loop siRNA expression system (stem expression system), antisense and sense code DNAs are arranged in the opposite direction, and these DNAs are connected via a linker DNA to construct a unit. A promoter is linked to one side of this unit to construct a stem-loop siRNA expression system. Herein, there is no particular limitation in the length and sequence of the linker DNA, which may have any length and sequence as long as its sequence is not the termination sequence, and its length and sequence do not hinder the stem portion pairing during the mature RNA production as described above. As an example, DNA coding for the above-mentioned tRNA and such can be used as a linker DNA.

In both cases of tandem and stem-loop expression systems, the 5′ end may be have a sequence capable of promoting the transcription from the promoter. More specifically, in the case of tandem siRNA, the efficiency of siRNA production may be improved by adding a sequence capable of promoting the transcription from the promoters at the 5′ ends of antisense and sense code DNAs. In the case of stem-loop siRNA, such a sequence can be added at the 5′ end of the above-described unit. A transcript from such a sequence may be used in a state of being attached to siRNA as long as the target gene silencing by siRNA is not hindered. If this state hinders the gene silencing, it is preferable to perform trimming of the transcript using a trimming means (for example, ribozyme as are known in the art). It will be clear to the skilled person that the antisense and sense RNAs may be expressed in the same vector or in different vectors. To avoid the addition of excess sequences downstream of the sense and antisense RNAs, it is preferred to place a terminator of transcription at the 3′ ends of the respective strands (strands coding for antisense and sense RNAs). The terminator may be a sequence of four or more consecutive adenine (A) nucleotides.

### Antibodies

Some aspects of the invention concern the use of an antibody or antibody-fragment that specifically binds to a polypeptide of the invention as defined above in the section entitled “inhibitor” and that is able to inhibit an activity of said polypeptide. Said antibody is designated as an inhibiting-antibody. Methods for generating antibodies or antibody-fragments that specifically bind to a given polypeptide are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

### Peptidomimetics

Peptide-like molecules (referred to as peptidomimetics) or non-peptide molecules that specifically bind to a polypeptide of the invention as defined above in the section entitled “inhibitor” or to its receptor polypeptide and that may be applied in any of the methods of the invention as defined herein (for example for altering the activity or steady state level of a polypeptide of the invention) as an antagonist or inhibitor of a polypeptide of the invention and they may be identified using methods known in the art per se, as e.g. described in detail in U.S. Pat. No. 6,180,084 which incorporated herein by reference. Such methods include e.g. screening libraries of peptidomimetics, peptides, DNA or cDNA expression libraries, combinatorial chemistry and, particularly useful, phage display libraries. These libraries may be screened for an antagonist of a polypeptide by contacting the libraries with a substantially purified polypeptide of the invention, a fragment thereof or a structural analogue thereof.

### Pharmaceutical Compositions

The invention further relates to a pharmaceutical preparation comprising as active ingredient an inhibitor as identified herein wherein said inhibitor is selected from the group consisting of: a polypeptide, a nucleic acid, a nucleic acid construct, a gene therapy vector and an antibody. All these ingredients were already defined herein. Said preparation or composition preferably comprises at least one pharmaceutically acceptable carrier in addition to an active ingredient.

In some methods, a polypeptide or antibody of the invention as purified from mammalian, insect or microbial cell cultures, from milk of transgenic mammals or other source is administered in purified form together with a pharmaceutical carrier as a pharmaceutical composition. Methods of producing pharmaceutical compositions comprising polypeptides are described in U.S. Pat. No.'s 5,789,543 and 6,207,718. The preferred form depends on the intended mode of administration and therapeutic application.

A pharmaceutical carrier can be any compatible, non-toxic substance suitable to deliver a polypeptide, antibody or gene therapy vector to a patient. Sterile water, alcohol, fats, waxes, and inert solids may be used as a carrier. A pharmaceutically acceptable adjuvant, buffering agent, dispersing agent, and the like, may also be incorporated into a pharmaceutical composition.

The concentration of a polypeptide or antibody of the invention in a pharmaceutical composition can vary widely, i.e., from less than about 0.1% by weight, usually being at least about 1% by weight to as much as 20% by weight or more.

For oral administration, an active ingredient can be administered in solid dosage forms, such as capsules, tablets, and powders, or in liquid dosage forms, such as elixirs, syrups, and suspensions. An active component or ingredient can be encapsulated in gelatin capsules together with an inactive ingredient and a powdered carrier, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable colour, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

A polypeptide, antibody or nucleic acid construct or gene therapy vector is preferably administered parentally or systemically. A polypeptide, antibody, nucleic acid construct or vector for preparations must be sterile. Sterilisation is readily accomplished by filtration through sterile filtration membranes, prior to or following lyophilisation and reconstitution. One preferred route of administration is systemic, more preferably orally. Another preferred route is a parental route for administration of A polypeptide, antibody, nucleic acid construct or vector is in accord with known methods, e.g. injection or infusion by subcutaneous, intravenous, intraperitoneal, intramuscular, intraarterial, intralesional, intracranial, intrathecal, transdermal, nasal, buccal, rectal, or vaginal routes. More preferably, the route for administration is intravenous or subcutaneous. A polypeptide, antibody nucleic acid construct or vector is administered continuously by infusion or by bolus injection. A typical composition for intravenous infusion could be made up to contain 10 to 50 ml of sterile 0.9% NaCl or 5% glucose optionally supplemented with a 20% albumin solution and 1 to 50 μg of the polypeptide, antibody nucleic acid construct or vector. A typical pharmaceutical composition for intramuscular injection would be made up to contain, for example, 1-10 ml of sterile buffered water and 1 to 100 μg of a polypeptide, antibody, nucleic acid construct or vector of the invention. Methods for preparing parenterally administrable compositions are well known in the art and described in more detail in various sources, including, for example, Remington's Pharmaceutical Science (15th ed., Mack Publishing, Easton, Pa., 1980) (incorporated by reference in its entirety for all purposes).

For therapeutic applications, a pharmaceutical composition is preferably administered to a cancer patient as earlier defined herein in an amount sufficient to reduce the severity of symptoms and/or prevent or arrest further development of symptoms. An amount adequate to accomplish this is defined as a “therapeutically-” or “prophylactically-effective dose”. Such effective dosages will depend on the severity of the condition and on the general state of the patient's health. In general, a therapeutically- or prophylactically-effective dose preferably is a dose, which is sufficient to reverse the symptoms, i.e. to prevent, delay and/or treat metastasis as earlier defined herein.

In the present methods, a polypeptide or antibody is usually administered at a dosage of about 1 μg/kg subject body weight or more per week to a subject. Often dosages are greater than 10 μg/kg per week. Dosage regimes can range from 10 μg/kg per week to at least 1 mg/kg per week. Typically dosage regimes are 10 μg/kg per week, 20 μg/kg per week, 30 μg/kg per week, 40 μg/kg week, 60 μg/kg week, 80 μg/kg per week and 120 μg/kg per week. In preferred regimes 10 μg/kg, 20 μg/kg or 40 μg/kg is administered once, twice or three times weekly. Treatment is preferably administered by parenteral route.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a polypeptide or a nucleic acid construct or an antibody or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

The invention is further illustrated by the following examples which should not be construed for limiting the scope of the present invention.

### DESCRIPTION OF THE FIGURES

FIG. 1. Gene-expression profiling of a metastasis model system identifies Fra-1 as a candidate metastasis gene. A. Phase contrast micrographs of RK3E and RIE rat epithelial cells expressing ligand-activated TrkB (‘RK3E^{TB}’ and ‘RIE^{TB}’ cells) or empty vector. Images were taken at 40× magnification B. Microarray gene-expression analysis of RK3E^{TB} and RIE^{TB} cells. The top 10 genes that are up- or down-regulated in both cell systems are shown in a heat map. C. Fra-1 expression levels measured by quantitative RT-PCR (upper panel) and western blotting (lower panel; n for PCR=3, error bars: S.D. Asterisk, different from control with P<0.01 based on a one-sided Student's t test). α-tubulin serves as loading control. D. Gel shift analysis measuring AP-1 DNA-binding activity. Supershift with Fra-1 antibody was performed to determine the relative contribution of Fra-1 to the total DNA-binding activity (empty arrows indicate supershifted AP-1 complex).

FIG. 2. Fra-1 is required for EMT of TrkB-expressing tumor cells. A. Fra-1 and E-cadherin expression levels measured by western blotting in RK3E^{TB} cells expressing independent shRNAs targeting Fra-1 as indicated. α-tubulin serves as loading control. B. Phase contrast micrographs showing the effects of Fra-1 depletion on cell morphology. Images were taken at 40× magnification C. Detection by immunofluorescence of Fra-1 and E-cadherin in cells as indicated. Phalloidin staining on cells plated in parallel is included to visualize the cytoskeleton. Parental RK3E cells are included as reference. D. Migration (upper panel) and invasion (lower panel) capacities as a function of Fra-1 depletion (n=3, error bars: S.D. Asterisk, different from control clones with P<0.001 based on a one-way ANOVA followed by LSD test). E. In vitro proliferation curve of RK3E^{TB} tumor cells, as a function of Fra-1 depletion (n=3, error bars: S.D.).

FIG. 3. Suppression of Fra-1 abrogates metastatic potential of TrkB-expressing primary tumors. A. In vivo growth curve of tumors formed by RK3E^{TB} tumor cells injected subcutaneously into nude mice, as a function of Fra-1 depletion (n=6, error bars: S.E.). B. Haematoxylin-Eosin staining of histological sections of subcutaneously expanding RK3E^{TB} tumors, as a function of Fra-1 depletion (scale bar: 100 μm; T: Tumor, S: skin). C. Macroscopic quantification of pulmonary metastases in mice carrying subcutaneous control or Fra-1-depleted RK3E^{TB} tumors, as analyzed at 3 weeks post-inoculation (microscopic quantification in Suppl. FIG. 2A). D. Representative images of macroscopic pulmonary metastases (left panels) and haematoxylin-eosin staining of histological lung sections (right panels, scale bar: 200 μm; M: metastasis) from mice described in C.

FIG. 4. Suppression of Fra-1 reverses EMT and blocks pulmonary colonization of human breast cancer cells. A. Expression levels of epithelial proteins as indicated in human MDA-MB-231 breast cancer cells as a function of Fra-1 depletion. α-tubulin serves as loading control. B. Detection by immunofluorescence of E-cadherin (upper panel) and cytoskeletal actin (by phalloidin staining; lower panel) of control and Fra-1-silenced MDA-MB-231 cells. C. In vitro proliferation curve of control and Fra-1-silenced MDA-MB-231 cells (n=3, error bars: S.D.). D. Images of the lungs (upper panel) and haematoxylin-eosin stained sections of the lungs (lower panel, scale bar: 100 μm; T: Tumor) of mice that were injected intravenously with 1.10⁶ MDA-MB-231 cells expressing independent Fra-1 shRNAs as indicated, photographed at 3 months after inoculation. E. Macroscopic quantification of the metastases formed by MDA-MB-231 cells described in D (n=5 lungs, error bars: S.D. Asterisk, different from control clones with P<0.001 based on a one-way ANOVA followed by LSD test). F. Immunohistochemical analysis of Fra-1 and Ki67 expression in lung tumors developing in mice inoculated intravenously with control or Fra-1-depleted MDA-MB-231 cells (inserts, higher magnification).

FIG. 5. Suppression of Fra-1 blocks metastasis from orthotopic human breast tumors. A. Quantification of the fluorescence in the lungs of nude mice inoculated intravenously with 1.10⁶ GFP-labeled LM2 cells expressing independent Fra-1 shRNAs as indicated, 35 days after inoculation (n=5 lungs, error bars: S.D. Asterisk, different from control with P<0.001 based on a one-way ANOVA followed by LSD test). B. Fluorescence imaging of the lungs of mice described in A. C. Quantification of the metastatic nodules in the lungs of nude mice injected in the 4^{th} mammary fat pad with GFP-labeled LM2 cells expressing independent Fra-1 sh-RNAs, 6 weeks after surgical removal of the primary tumor (n=10). D. Fluorescence imaging of the lungs of mice described in C.

FIG. 6. A Fra1-associated gene-expression profile accurately predicts clinical outcome of human breast cancer. A. Outline of the procedure used to generate a gene-expression profile that is associated with Fra-1 function and based on the Fra-1-dependent transcriptome in LM2 cells. B. Distant Metastasis-Free Survival (DMFS) of patients from the NKI295 data set (left panel) and Breast Cancer Specific Survival (BCSS) of patients from the Affymetrix validation set (Right panel) that were classified as having a ‘poor’ prognosis (blue line) or ‘good’ prognosis (black line) using the Fra-1 classifier. (Displayed p-values are based on the log-rank test).

FIG. 7. Fra-1 depletion in RK3E^{TB} cells reverts morphological transformation. A. Fra-1 expression levels in polyclonal pools of RK3E^{TB} cells containing empty vector or independent shRNAs targeting Fra-1, as indicated. B. Phase contrast micrographs of the cells described in B. C. Restoration of Fra-1 expression in sh-Fra1 (1) expressing RK3E^{TB} cells, resulting in morphological transformation. D. Phase contrast micrographs of the cells described in C. In both A. and C., the panels were taken from a single blot and α-tubulin serves as loading control. Cells were photographed at 40× magnification.

FIG. 8. Fra-1 is essential for TrkB-driven metastasis A. Microscopic quantification of pulmonary metastases disseminated from RK3E^{TB} tumors, as a function of Fra-1 depletion. The total number of metastases in 8 independent sections per mouse is indicated, representative of 3 independent experiments (asterisk, see Suppl. FIG. 2e). B. Haematoxylin-Eosin staining of a histological section of the lungs of a mouse that received a subcutaneous inoculation with Fra-1-depleted RK3E^{TB} tumor cells, displaying a micrometastasis (arrowhead) that failed to extravasate from pulmonary vessels (scale bar: 100 μm).

FIG. 9. Fra-1 is commonly overexpressed in human breast cancer cell lines. Western-blot analysis of Fra-1 expression in human breast cancer cell lines. β-actin serves as loading control.

FIG. 10. Fra-1 is required for lung metastasis of human breast cancer cells. A. Flow-cytometric analysis of GFP signal intensity in control and Fra-1-silenced LM2 cells prior to in vivo inoculation. B. Quantification of the fluorescence in the lungs of the mice inoculated intravenously with 1.10⁵ GFP-labeled LM2 cells expressing independent Fra-1 shRNAs as indicated, 35 days after inoculation (n=5 lungs, error bars: S.D. Asterisk, different from control with P<0.001 based on a one-way ANOVA followed by LSD test). C. Representative immunofluorescence imaging of the lungs of mice described in B. D. Expression levels of epithelial proteins in LM2 cells as a function of Fra-1 depletion. E. Weight of the orthotopic LM2 tumors upon surgical removal after one month of growth, as a function of Fra-1 depletion.

FIG. 11. Identification of Fra-1-regulated genes essential for lung metastasis formation. A. Quantification of the fluorescence in the lungs of the mice inoculated intravenously with 1.10⁵ GFP-labelled LM2 cells expressing independent shRNAs directed against the Fra-1-regulated genes as indicated, 5 weeks after inoculation (n=3 lungs, error bars: S.D. Asterisk, different from control for the 2 independent sh-RNAs with P<0.05 based on an unpaired one-sided t test). B. Expression levels of the Fra-1-regulated genes measured by quantitative RT-PCR in the LM2 cells injected into mice. C. Representative immunofluorescence imaging of the lungs of mice described in A.

### EXAMPLES

All references cited herein are incorporated by reference.

### Gene Expression Profiling of a Metastasis Model System Identifies Fra-1 as a Candidate Metastasis Gene

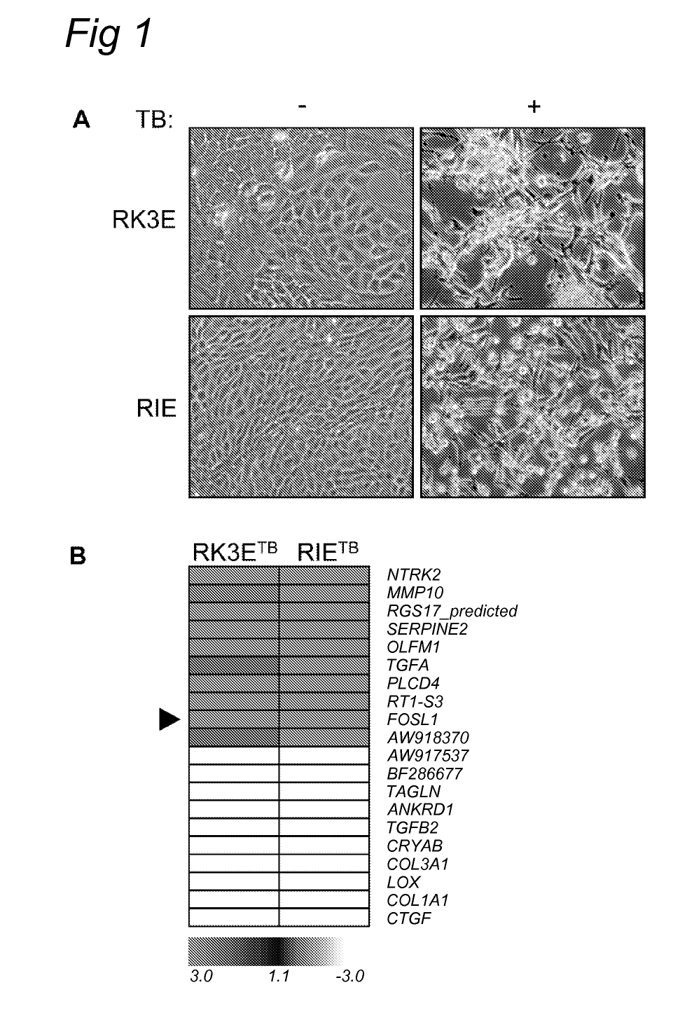

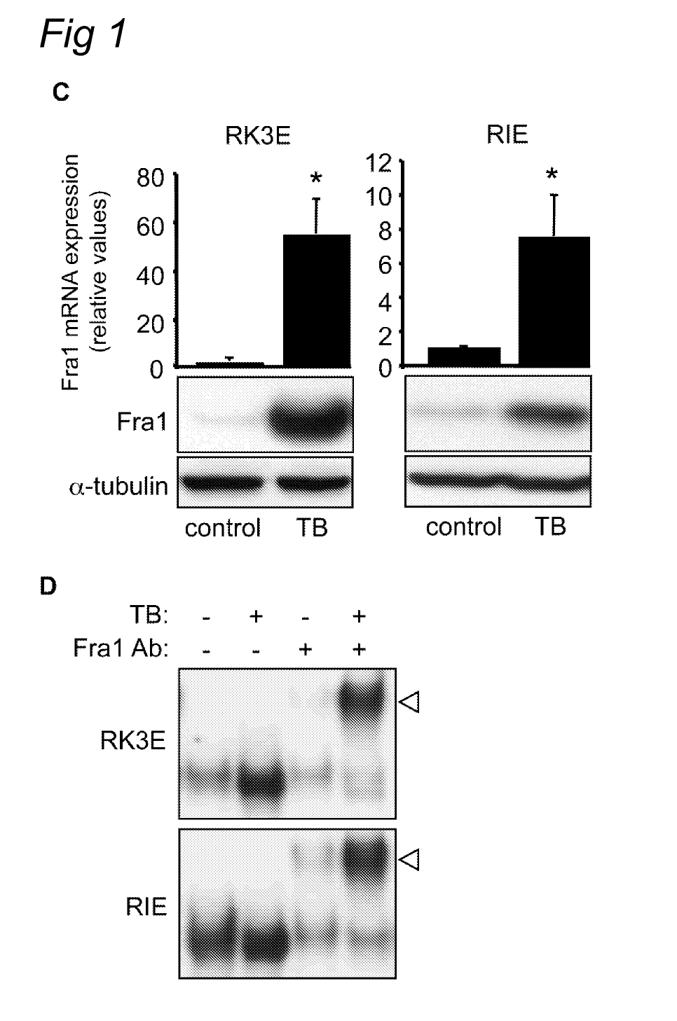

We performed microarray gene-expression profiling on both RIE and RK3E cells ectopically expressing TrkB and BDNF (hereafter RIE^{TB} or RK3E^{TB} cells; FIG. 1A). Commonly regulated outliers were considered potential metastasis promoters or inhibitors (the raw data of the microarray analyses are available at http://www.ebi.ac.uk/microarray-as/aer/#ae-main[0], accession E-NCMF-21). Among several other potentially interesting outliers was FOSL1 (FIG. 1B). Confirming our microarray expression data, Fra-1 was upregulated to up to >50-fold by activated TrkB at both the transcriptional and protein levels (FIG. 1C). Gel shift experiments revealed that this caused Fra-1 to become a major component of AP-1 DNA-binding complexes, also in both cell systems (FIG. 1D). Fra-1 upregulation in this setting is most probably due to Ras activation, acting as a downstream effector of TrkB signaling (Carter et al., 1995).

### Suppression of Fra-1 Reverses EMT and Abrogates Metastatic Potential of TrkB-Expressing Primary Tumors

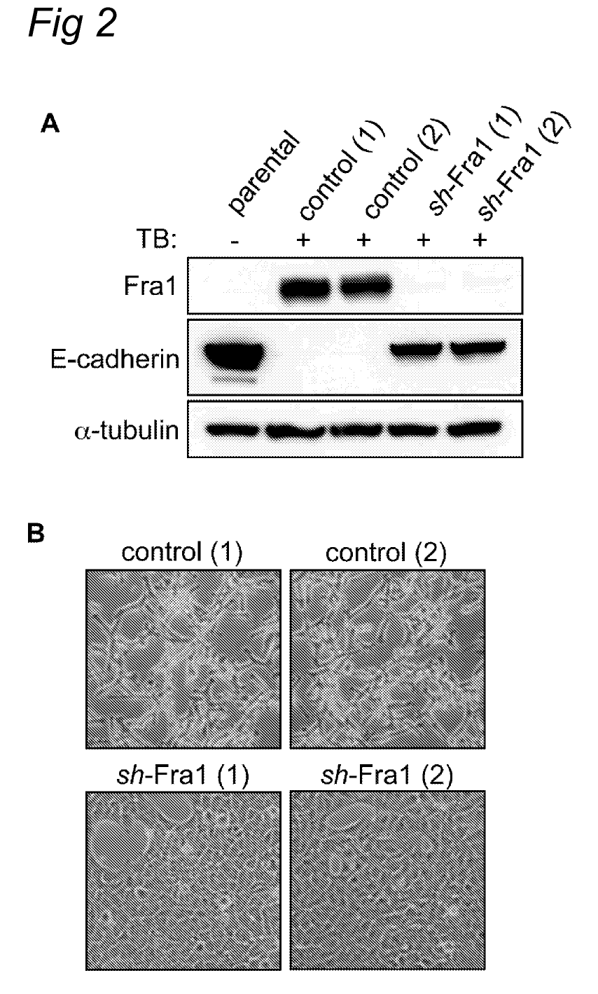

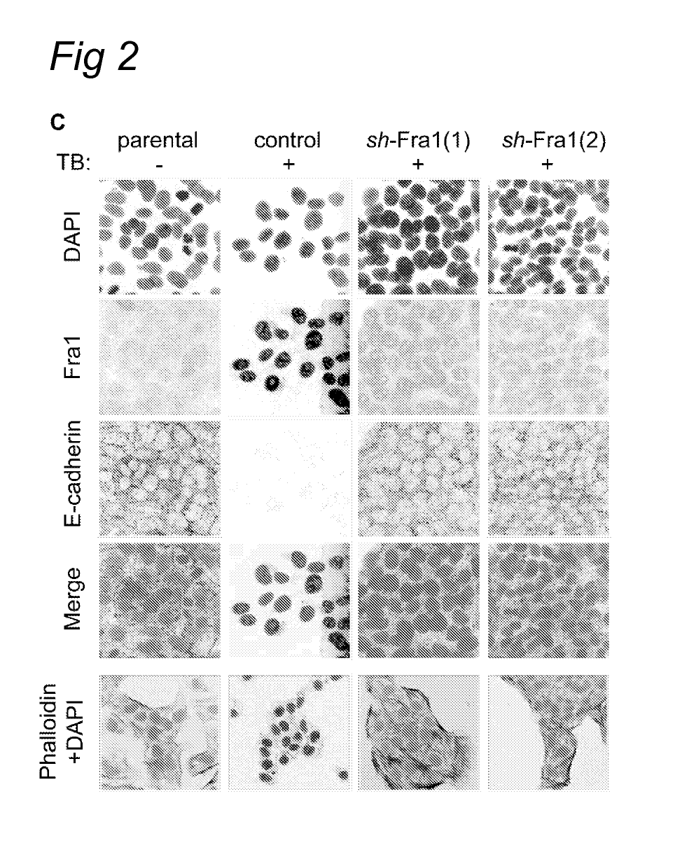

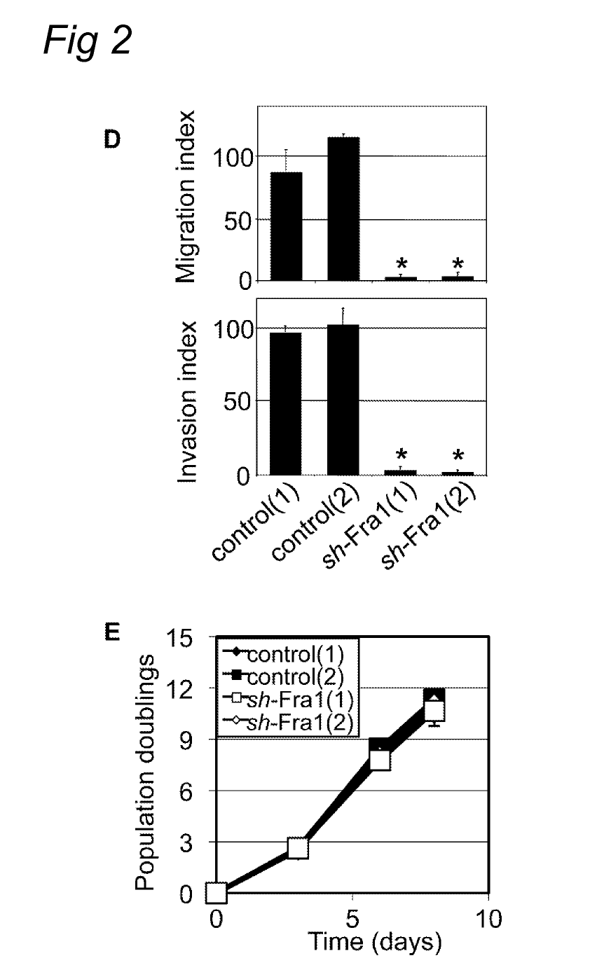

To address the functional relevance of Fra-1 in oncogenic transformation and metastasis, we depleted it from RK3E^{TB} cells using retroviral vectors encoding independent short-hairpin (sh) RNAs (sh-Fra-1(1) and (2)). Several clonal cell populations were established in which the Fra-1 protein levels were reduced back to those seen in parental cells (FIG. 2A and data not shown).

Analysis performed in vitro revealed that Fra-1 silencing completely reversed the induction by activated TrkB of a spindle-like cellular phenotype, and cells regained a typical epithelial ‘cobble-stone’ morphology with extensive cell-cell junctions and actin cytoskeleton reorganization (FIG. 1A, 2B, C). This was observed for cell clones expressing non-overlapping shRNAs, as well as in polyclonal sh-Fra-1 cell pools (Suppl. FIG. 1A, B). Furthermore, restoration of Fra-1 expression reverted the effect of Fra-1 silencing (Suppl. FIG. 1C, D), all strongly arguing against an RNAi off-target effect. These morphological rearrangements were highly reminiscent of a reversion of EMT. Supporting this notion, Fra-1 depletion fully restored expression and correct subcellular localization of E-cadherin (FIG. 2A, C). This was paralleled by a complete cancellation of both the migratory and invasive properties of RK3E^{TB} cells (FIG. 2D).

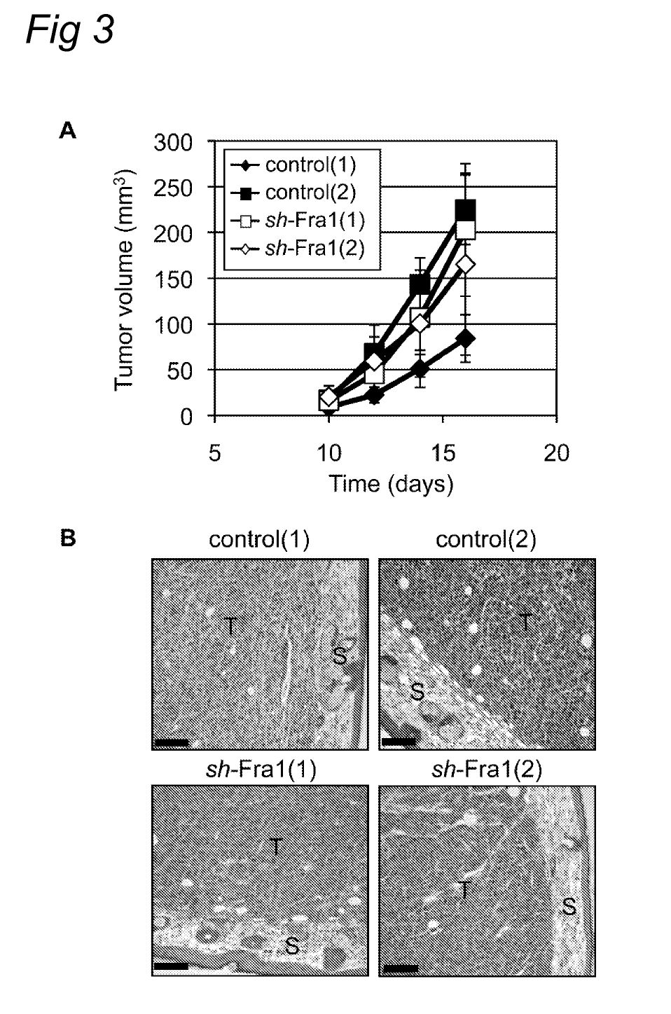

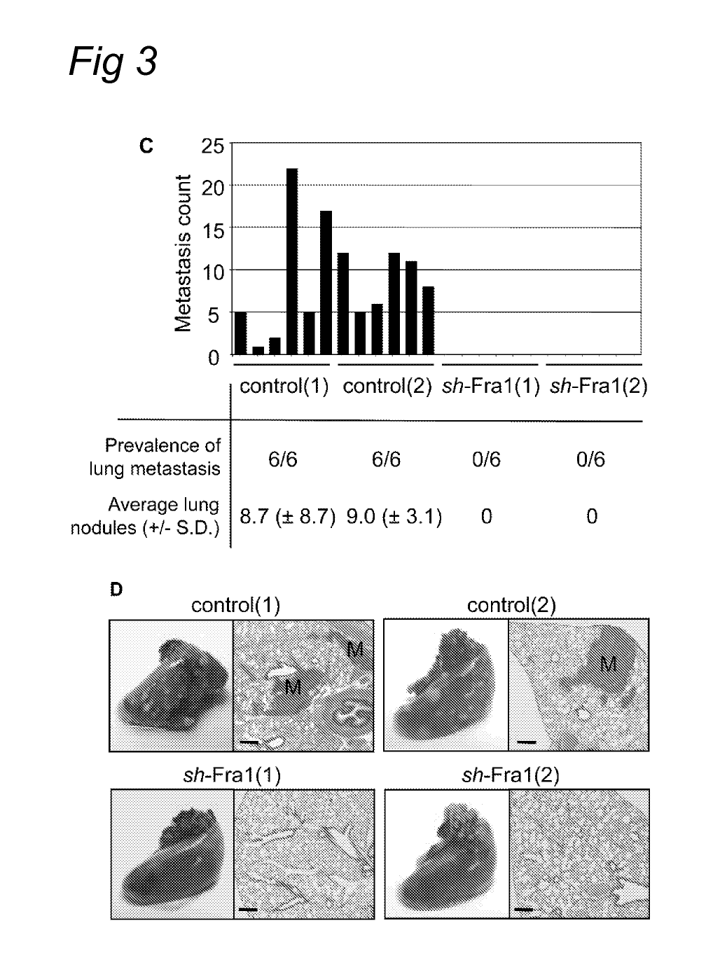

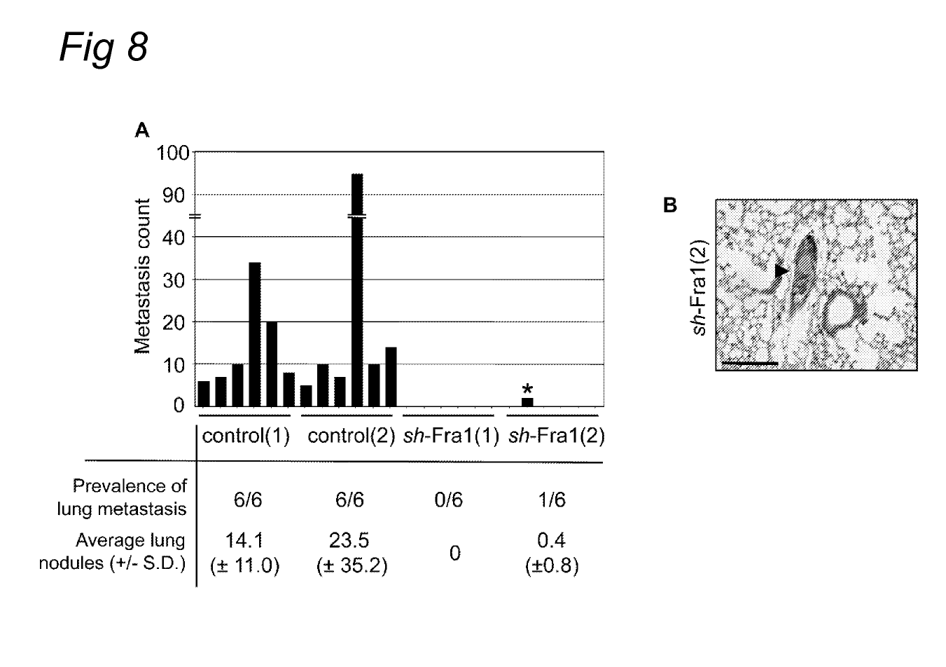

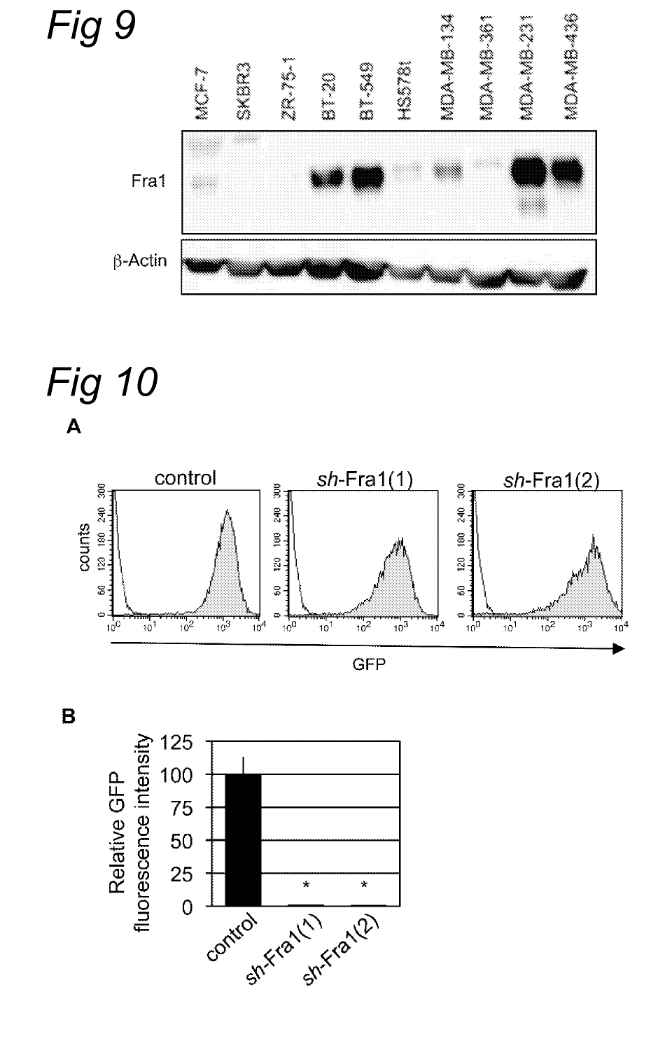

Importantly, in spite of these effects, Fra-1 depletion did not at all influence the proliferative activity of RK3E^{TB} cells in culture (FIG. 2E). Similarly, when inoculated subcutaneously into athymic nude mice, cells silenced for Fra-1 produced tumors that expanded as rapidly as control tumors, and with indistinguishable morphology (FIG. 3A, B). Next, we addressed whether Fra-1 corresponds to an important determinant of the metastatic potential of these tumor cells in xenograft experiments. All mice that had received control tumor cells developed macroscopically detectable lung metastases that had disseminated from the subcutaneous primary tumors (FIG. 3C, D). In striking contrast, none of the mice injected with Fra-1-silenced tumor cells displayed any visible metastases. Microscopic histological examination of the lungs confirmed the presence of metastatic lesions invading the lung parenchyma in all mice that had received control cells, whereas pulmonary metastases were virtually absent in the recipients of Fra-1-silenced tumor cells (FIG. 8A). In fact, in the lungs of 36 mice carrying Fra-1-depleted tumors, only two colonies were observed, comprising a few cells only that had failed to extravasate from the pulmonary vessels (FIG. 8B). These results demonstrate that, at least in the context of TrkB-driven rodent epithelial tumor cell metastasis, Fra-1 is dispensable for primary tumorigenesis, but is crucial for the ability of these tumors to produce distant metastases from primary tumors.

### Suppression of Fra-1 Reverses EMT and Blocks Pulmonary Colonization of Human Breast Cancer Cells

Fra-1 is frequently overexpressed in human solid tumors, including those derived from breast, colon, thyroid tissue and in mesothelioma, as well as in many cell lines derived from various human tumor types (reviewed in Milde-Langosch, 2005). In a microarray gene-expression analysis, a correlation was noted between Fra-1 expression levels and the in vitro invasive potential of human breast cancer cell lines (Zajchowski et al., 2001). Also exclusively in vitro, Fra-1 overexpression in weakly invasive breast tumor cells has been shown to increase their invasive potential, while silencing of Fra-1 in a highly invasive cell line decreased it (Belguise et al., 2005). Although these results raise the possibility for a role for Fra-1 in metastasis of human mammary carcinoma cells also in vivo, this has not yet been addressed.

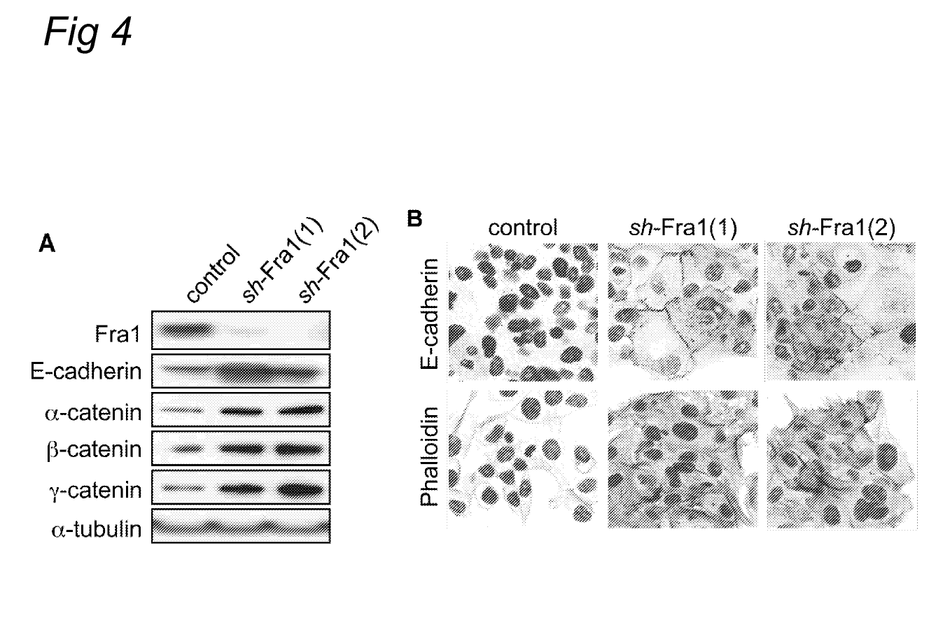

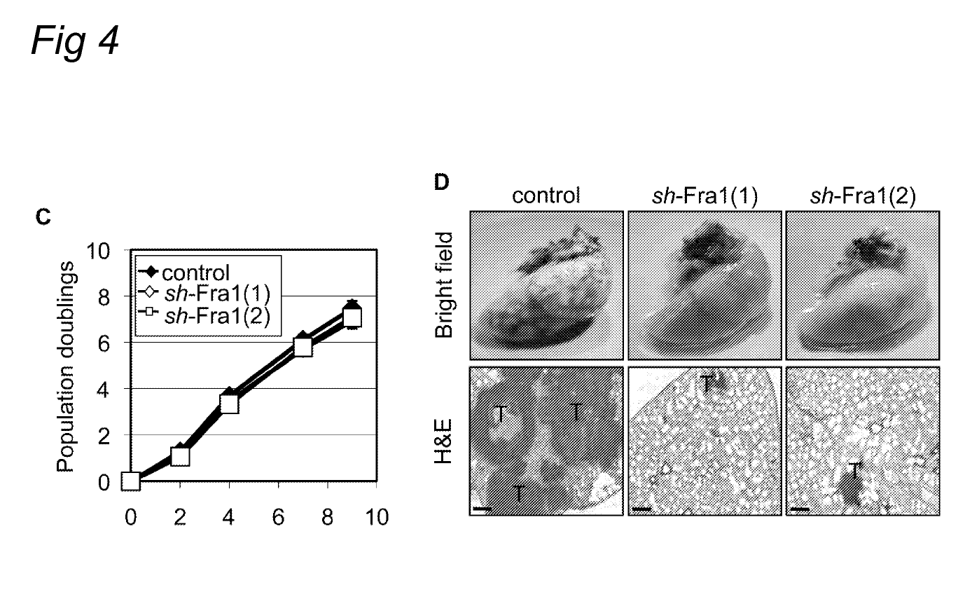

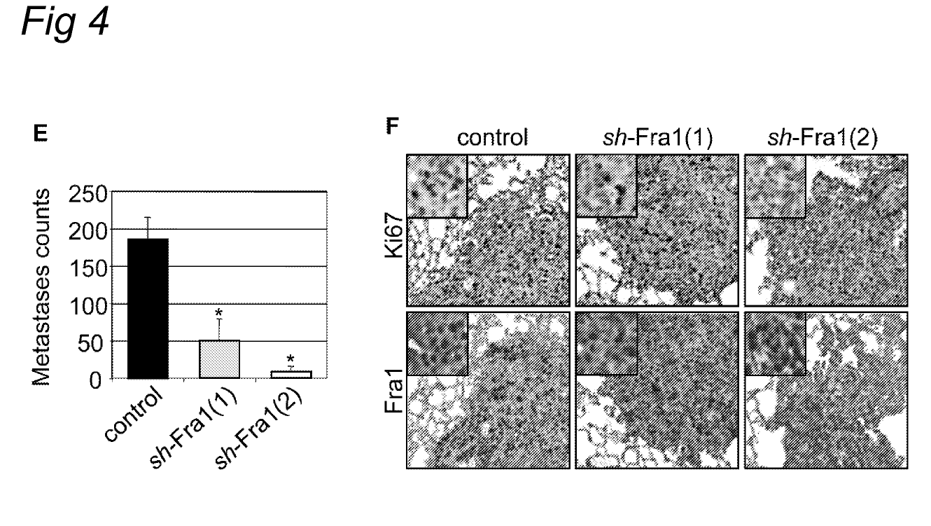

To study any role of Fra-1 in the oncogenic and metastatic potential of human breast cancer cells in vivo, we depleted its RNA from MDA-MB-231 cells, which strongly overexpress Fra-1 (Belguise et al., 2005 and Suppl. FIG. 3), by lentiviral transduction of either of two non-overlapping shRNAs. Similar to what we had observed for the rodent cell system, silencing of Fra-1 in polyclonal cell populations led to a strong upregulation of E-cadherin expression, as well as other epithelial proteins (FIG. 4A). Concomitantly, silencing of Fra-1 caused E-cadherin to relocalize at the cell membrane (FIG. 4B, upper panel). Together, these results indicate that Fra-1 is required for downregulation of epithelial characteristics in both rodent tumor cells and human mammary carcinoma cells. Consistent with previous findings (Belguise et al., 2005; Vial et al., 2003), this was associated with extensive cytoskeletal reorganization (FIG. 4B, lower panel).

Importantly, similar to what we observed for the rodent cells, the proliferative potential of MDA-MB-231 cells was not at all affected by the silencing of Fra-1 (FIG. 4C). This is in contradiction with previous findings (Belguise et al., 2005) and may be explained by our use of stably integrated proviral shRNA as opposed to their use of transfected siRNA. To examine if, similar to the rodent cell system, Fra-1 has a crucial role in the capacity of human breast cancer cells to colonize the lungs, MDA-MB-231 cells were inoculated intravenously into athymic mice. Indeed, Fra-1 depletion caused a strong decrease in the tumor burden in the lungs (FIG. 4D, E). The rare pulmonary tumors of Fra-1-depleted MDA-MB-231 cells that did emerge expressed normal levels of Fra-1 and proliferated as fast as the colonies formed by control cells (FIG. 4F). Because we used polyclonal pools of Fra-1 depleted cells, these tumors most probably emerged from cells with incomplete silencing of Fra-1 (similar to what has previously been observed for Twist—Yang et al., 2004). These results indicate that depletion of Fra-1 from human breast cancer cells restores key epithelial characteristics and blocks their ability to colonize the lungs.

Suppression of Fra-1 Blocks Metastasis from Orthotopic Human Breast Tumors

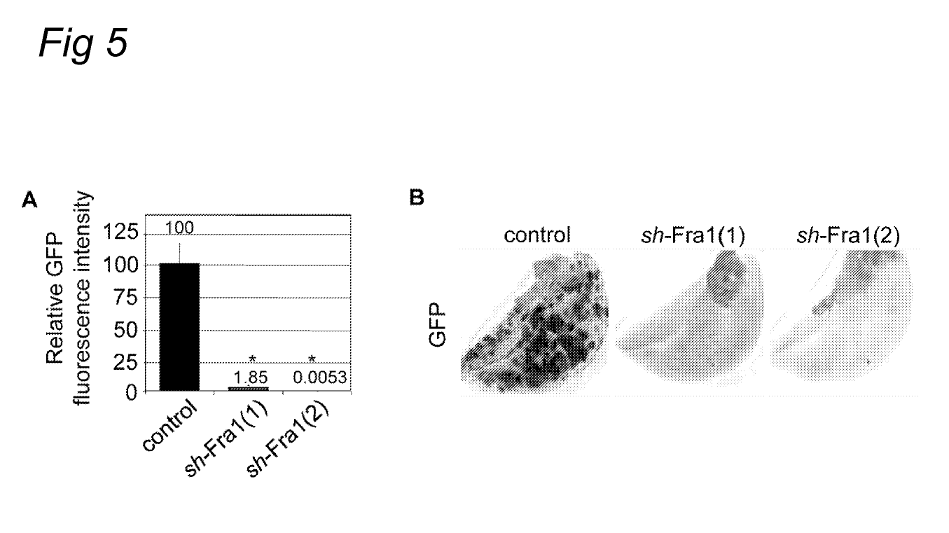

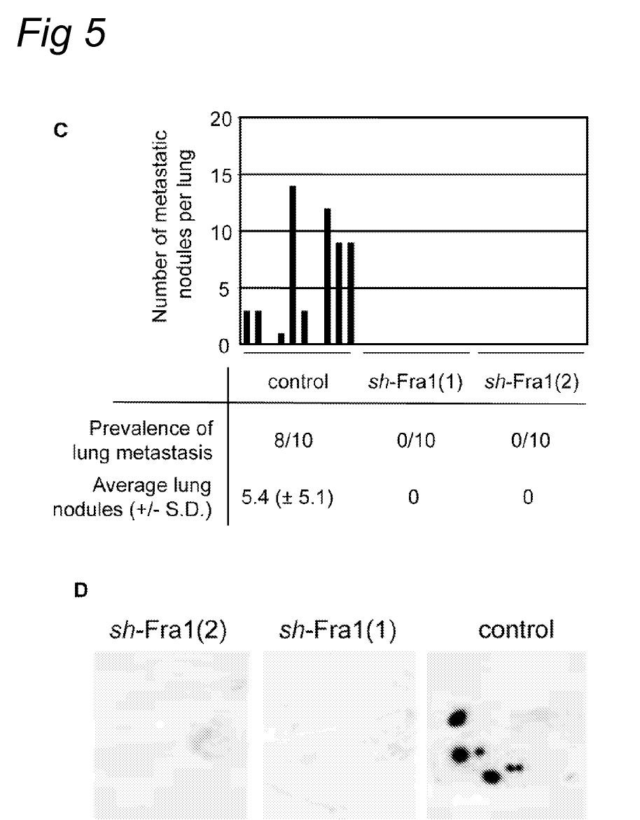

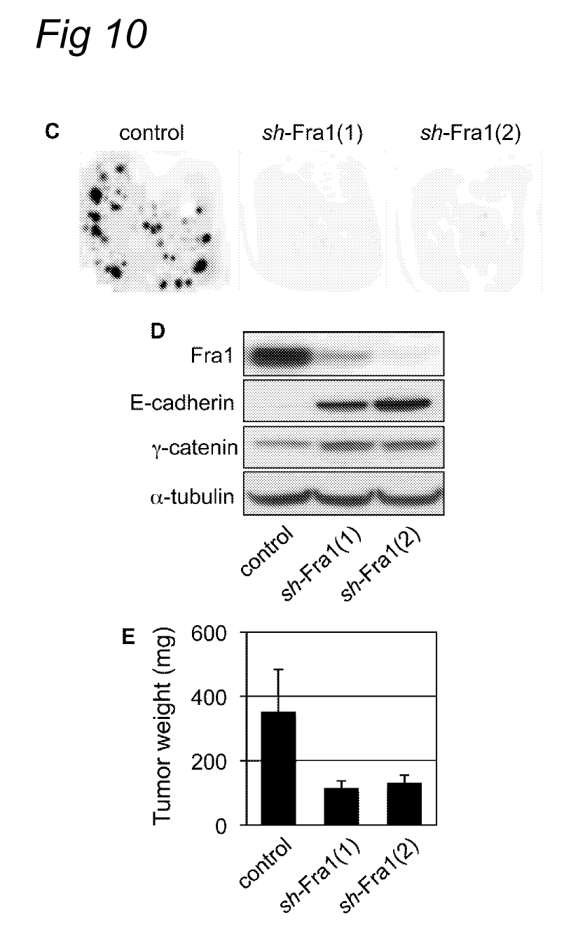

As an independent measure, we inoculated intravenously 1.10⁶ GFP-labeled LM2 cells, an MDA-MB-231-derived cell line associated with a high proclivity to metastasize to the lungs (Minn et al., 2005). Flow cytometry just prior to injection showed that all cell lines expressed equal levels of GFP, irrespective of Fra-1 silencing (Suppl. FIG. 4A). Fluorescence imaging of the lungs revealed an almost 20,000-fold reduction in the tumor burden upon silencing of Fra-1 (FIG. 5A, B). A similarly strong suppression of metastasis by Fra-1 depletion was observed with inoculation of 1.10⁵ cells (Suppl. FIG. 4B, C). The reduction in lung colonization was dose-dependent, as illustrated by a correlation that was observed between Fra-1 knockdown levels and the colonizing capacity of these cells (with sh-Fra-1(2) performing best in both settings; compare FIG. 5A and FIG. 10B to FIG. 10D). Together, these results reveal a crucial role for Fra-1 in the capacity of human breast cancer cells to form experimental pulmonary metastases.

As intravenous inoculation bypasses the need for tumor cells to invade and intravasate, we next determined whether Fra-1 is required also for the full metastatic cascade. For this, we used an orthotopic model in which GFP-labeled LM2 cells were injected into the mammary fat pad of nude mice. Cells receiving the control plasmid developed primary tumors that metastasized to the lungs in most of the animals (FIG. 5C, D). By contrast, LM2 cells expressing shRNAs against Fra-1 developed tumors that grew more slowly (FIG. 10E) and were unable to develop detectable lung metastases (FIG. 5C, D). Again, the inhibition of lung-colonizing tumor activity was associated with upregulation of E-cadherin expression (FIG. 10D). Thus, together these results reveal a crucial role for Fra-1 in the capacity of human breast cancer cells to form pulmonary metastases from primary tumors. As we show that this block in metastasis is seen also for intravenously inoculated tumor cells, these data also indicate that Fra-1 fulfills an essential role in the late steps of the metastatic process.

### Fra-1 Expression and its Associated Gene-Expression Profile are Determinants of Breast Cancer Recurrence

Further supporting the crucial role of Fra-1 in the ability of human breast cancer cells to metastasize, we observed an association between Fra-1 mRNA expression levels in primary human breast carcinomas and the risk of developing distant site metastasis in the Affymetrix training set, a cohort of 509 breast cancer patients (p=0.03; log-rank test, see Methods). The fact that Fra-1 corresponds to a transcription factor raised the possibility that its associated transcriptome, too, is endowed with prognostic power regarding clinical outcome. In fact, it has been suggested that in a so-called data-driven approach for finding connections between gene-expression patterns and tumor behavior, the target genes of transcription factors often represent better biomarkers than the transcription factor itself, as they are expressed as a function of the activity—and not just expression—of the transcription factor (van 't Veer and Bernards, 2008).

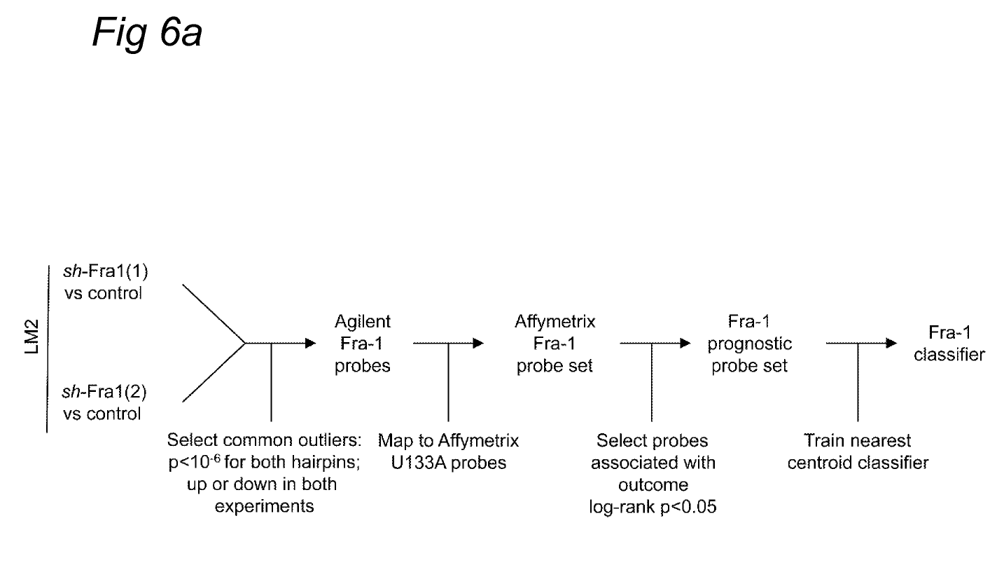

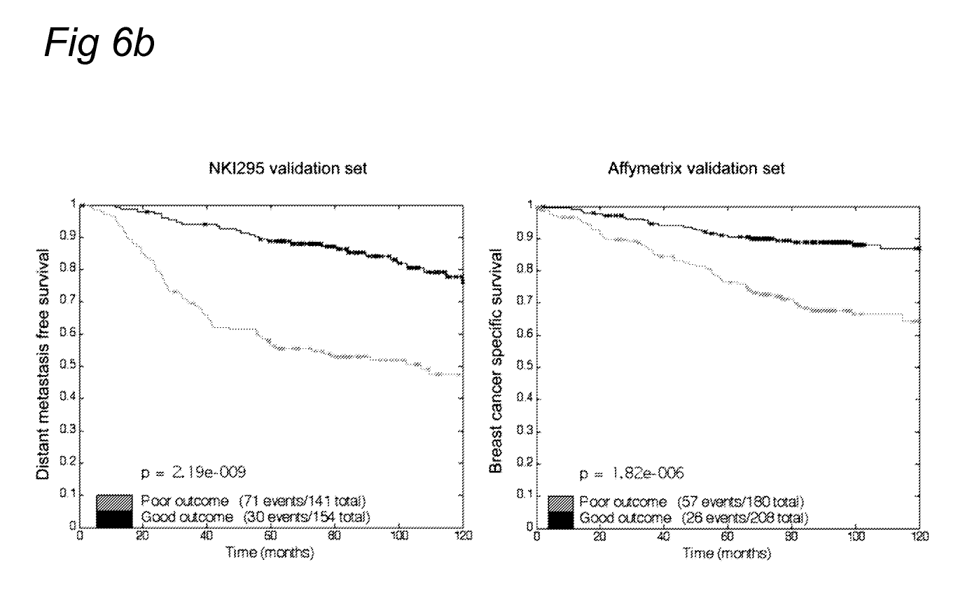

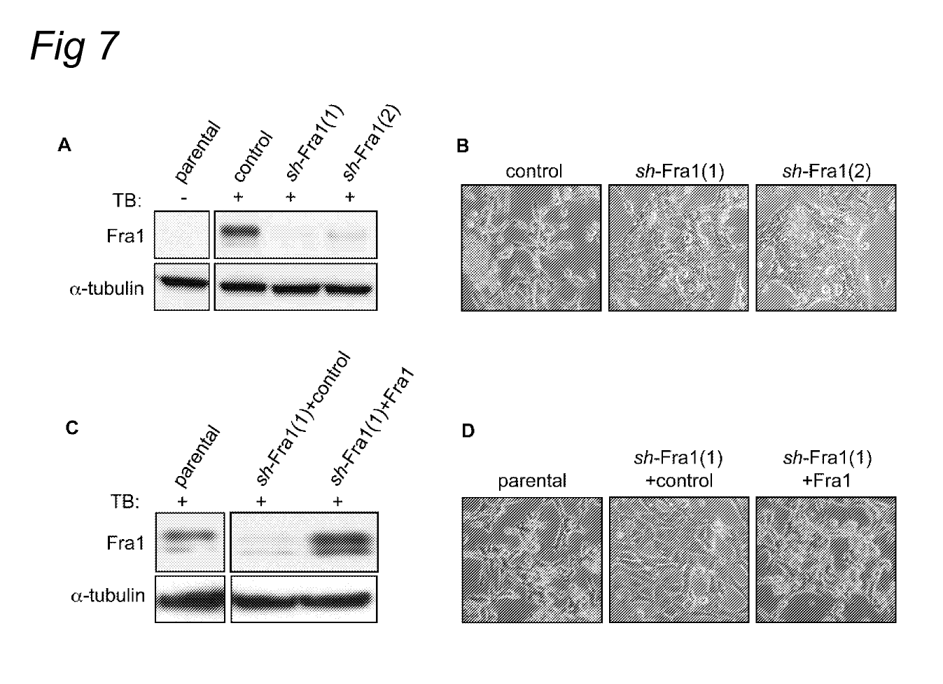

To determine whether Fra-1 target gene expression levels correlate with breast cancer recurrence, we performed microarray gene-expression profiling of LM2 cells in which Fra-1 was silenced using shRNA. Probes that were significantly up- or down-regulated by both shRNAs (p<1.10⁻⁵) were selected (not shown). This set of Agilent probes was mapped to the corresponding Affymetrix probes. Probes showing prognostic value in the Affymetrix training set (509 patient cohort) were then used to generate the Fra-1 classifier, which contained 447 probes (FIG. 6A, and not shown).

The Fra-1 centroid classifier was validated independently on two series of human breast cancer gene-expression profiles: the Affymetrix validation set (a set derived from curated available datasets) and the NKI295 dataset (see Methods for details on the composition of these sets). Remarkably, the difference in survival between good and poor prognosis groups as defined by this Fra-1 classifier was highly significant in both series, with p=2.19×10⁻⁹ (log-rank test, DMFS as endpoint) on the NKI295 set and p=1.82×10⁻⁶ (log-rank test, BCSS as endpoint) on the Affymetrix validation set (FIG. 6B). In a multivariate Cox analysis, the Fra-1 classifier remained an independent predictor in the presence of known clinical predictors, including lymph node status, size of the tumor, estrogen receptor status, and Elston-Ellis grading in the 295 patients from the NKI (Table 1). A classifier containing 445 probes was generated using similar procedures in another breast cancer cell line (MDA-MB-231 cells), with similar outcome.

Interestingly, 188 probes, mapping to 168 different genes, were common to both the 447 probes set derived from LM2 cells and the 445 probes set derived from MDA-BM-231 cells (Table 2). Those genes potentially comprise gene products causally involved in metastasis. In this respect, inhibiting gene products whose expression is higher in poor prognosis tumors than in good prognosis tumors may result in inhibition of metastasis development.

### A Systematic Analysis of Fra-1-Regulated Genes Identifies 12 Genes Essential for Metastasis of Human Breast Cancer Cells

We then aimed to use the Fra-1 classifiers as a platform to search for Fra-1-regulated genes critically involved in the metastatic activity of human breast cancer cells. Among the 168 genes common between the LM2 and MDA-MB-231 classifiers, we selected the genes that are commonly down-regulated by two sh-RNAs targeting Fra-1 in both cell system, suggesting that the expression of those genes is activated, whether directly or indirectly, by Fra-1. Among those genes, we then selected those that are highly expressed specifically in poor prognosis breast cancer patients, since they correspond to the genes whose overexpression may contribute to metastasis formation. This strategy has yielded a list of 31 genes. The majority of these genes have been shown to play a role in cancer (progression). Importantly, one of them, the Metadherin gene, has recently been shown to be essential for the metastatic dissemination of breast cancer cells to the lungs (Hu et al., 2009), validating this approach.

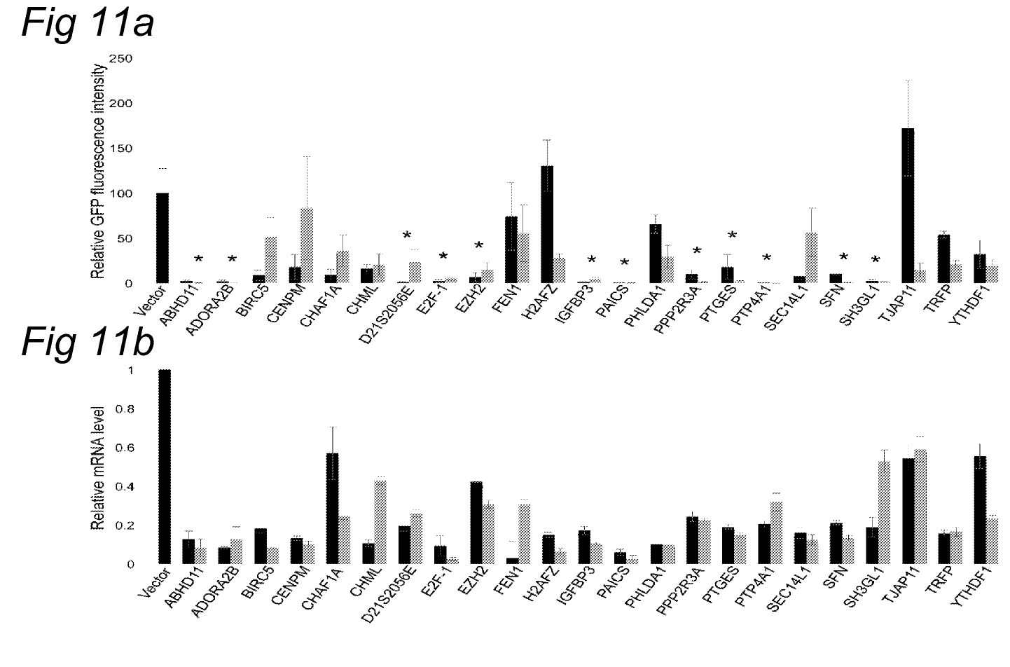

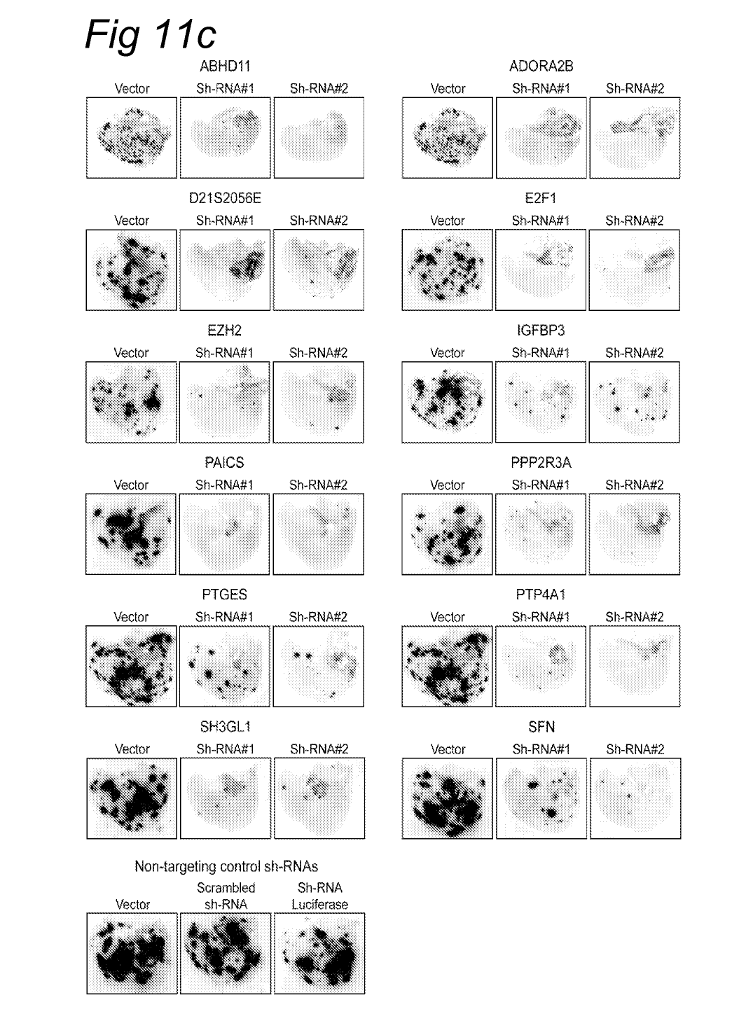

We then systematically tested the effect of the silencing of each of these 31 genes in LM2 cells on metastasis formation in vivo. For this purpose, we inhibited the expression of each of these genes using at least 2 independent sh-RNAs, using lentiviral-mediated delivery. We tested those stably modified cell lines for pulmonary metastases formation in vivo after intravenous injection of the cells into nude mice. We used GFP-labeled cells and we then quantified metastasis formation in the lungs of mice by imaging of the GFP fluorescence 5 weeks after injection of the cells (FIG. 11). After statistical analysis, we identified twelve genes whose inhibition significantly suppressed metastasis (table 6), and many did so in a dramatic way.

Importantly, some of these genes encode enzymes, receptors or other proteins that appear to be druggable. For some of these, a small molecule inhibitor is already known and available (Table 4). We are now investigating the effects of these pharmacological inhibitors on metastasis formation in vivo. We are also repeating these experiments for the twelve positive genes using an independent experimental system, a luciferase in vivo imaging system, in order to further validate and extend these observations and to provide additional information on the kinetics and mechanism of the block in metastasis formation. Finally, we are investigating the effect of these twelve genes on primary tumor growth after orthotopic injection in the mammary fat pad of mice, to determine if their effect is specific for metastasis or also applies to primary tumor development.

### DISCUSSION

Metastatic spread of tumor cells accounts for most of cancer mortality, yet few of its key players have been uncovered. To identify targets for therapeutic intervention, it is imperative to resolve the molecular processes underlying metastasis. By combining genetic and functional analyses with RNA interference in a metastasis model system, we demonstrate that the transcription factor Fra-1 is strictly required for metastatic tumor cell dissemination. Correspondingly, it is overexpressed in several human tumors, including breast cancers. We show that Fra-1 depletion from human breast cancer cells had a dramatic impact on their ability to metastasize to the lungs from primary orthotopic tumors.

Many early oncogenic alterations, such as Ras mutations or overexpression of receptor tyrosine kinases, lead to the activation of the MAP kinase pathway and its downstream effector transcription factors. Among them are the members of the Fos (c-Fos, FosB, Fra-1 and Fra-2) and Jun (c-Jun, JunB, JunD) families of transcription factors, which are involved in the formation of AP-1 complexes (Eferl and Wagner, 2003). Fos and Jun proteins are established oncogenes (Eferl and Wagner, 2003), and Fra-1 has been shown to contribute to cell transformation or tumorigenesis in several settings (Adiseshaiah et al., 2007; Ramos-Nino et al., 2002; Vallone et al., 1997). We observed that Fra-1 depletion had little impact on the proliferative activity of human breast cancer cells in vitro and in vivo. In contrast, Fra-1 was strictly required for metastasis development in both rat and human tumor cells. These results suggest that Fra-1, at least in these two independent experimental settings, contributes relatively more to metastasis than it does to primary tumor growth. Although this does not preclude a contribution to early tumorigenesis as well, Fra-1 appears to behave as a tumor progression, rather than tumor-initiating, factor.

In addition to its implication in cell transformation, the role of Fra-1 in tumor cell invasion and migration has gained increasing interest over the years (Ozanne et al., 2006). However, the in vivo relevance of these data, namely whether Fra-1 corresponds to an important determinant in the complex cascade of events ultimately leading to metastasis has never been addressed. In vitro, Fra-1 has been shown to mediate cell motility or invasion (Adiseshaiah et al., 2007; Belguise et al., 2005; Vial et al., 2003). These results are in line with our observation that inhibition of metastasis upon Fra-1 silencing was associated with a reversion of cellular actin organization and a reduction in cell migration and invasion. We show here that Fra-1 silencing also induced re-expression and correct subcellular localization of E-cadherin, providing evidence that Fra-1 is causally involved in E-cadherin downregulation in breast cancer cells. This is consistent with the finding that Fra-1 expression levels negatively correlate with E-cadherin expression in breast cancer cell lines (Zajchowski et al., 2001). Interestingly, this regulation appears to be mutual, as in turn, induction of EMT through inhibition of E-cadherin function upregulates Fra-1 expression (Andersen et al., 2005). Suppression of E-cadherin expression denotes a central event in EMT and is associated with tumor invasiveness, metastatic dissemination and poor patient prognosis (Thiery and Sleeman, 2006). While evidence of abundant EMT in clinical breast cancer specimens has been lagging (Hugo et al., 2007), there is ample experimental evidence supporting an intimate role for EMT in allowing tumor cells to disrupt cell-cell contacts, become anoikis-resistant and invade, all contributing to the metastatic process (Christofori, 2006; Liotta and Kohn, 2004; Thiery and Sleeman, 2006; Yang and Weinberg, 2008). Interestingly, it has recently been demonstrated that human stem-like breast cancer cells express markers associated with EMT (Mani et al., 2008). Our results suggest that Fra-1 acts as a central determinant in breast cancer metastasis, at least in part, by acting as a critical regulator of EMT.

Extending our identification of Fra-1 as an important contributor to metastasis, we demonstrate that a Fra-1-dependent transcriptome, which is based on Fra-1-depleted human breast carcinoma cells, is associated with high prognostic power in human breast cancer. The classifier presented here thus functionally connects prognostic power in breast cancer recurrence with a defined set of genes whose expression is regulated by a single transcription factor that is functionally validated as a causal factor for the disease. As such, it highlights the importance of Fra-1 in breast cancer metastasis in human patients and presents us with a new tool for patient stratification. As it is directly connected to the functional properties of Fra-1 in metastasis, the classifier may also contain candidate target genes that can be exploited for therapeutic intervention. Our data merit efforts to inactivate Fra-1 or components of its signaling pathway for clinical intervention of human breast cancer and several other cancers in which its expression is increased.

### Methods

### Vectors and Antibodies

RIE (gift from R. D. Beauchamp, Nashville, Tenn., and K. D. Brown, Cambridge, UK) and RK3E (ATCC) cells were retrovirally transduced with murine TrkB and BDNF expression constructs as previously described (Douma et al., 2004), except that the TrkB cDNA was subcloned into pMSCV-blasticidin. BDNF (N-20), Fra-1 (R-20), and Trk (C-14) antibodies were from Santa Cruz, α-catenin (610193), β-catenin (14), γ-catenin (610253) and E-cadherin (610181) antibodies were from Becton Dickinson. α-tubulin antibody (DM1A) was from Sigma. Ki67 antibody (MM1) was from Vision Biosystems. Phospho-Smad2 (3101) antibody was from Cell Signaling Technologies.

### Cell Culture

RIE-1 cells, RK3E cells, MDA-MB-231 cells (gift from L. Smit, Amsterdam) and LM2 cells (subline#4173, gift from Prof. J. Massague, New York) were cultured in DMEM (Life Technologies) supplemented with 10% FCS (Greiner bio-one), 2 mM glutamine, 100 units mi⁻¹ penicillin, and 0.1 mg mi⁻¹ streptomycin (all Gibco). To measure cell proliferation rates, cells were seeded at 3.10⁵ (RK3E) or 1.10⁶ (MDA-MB-231) per 100-mm dish. For each cell line, cells from three dishes were trypsinized and counted every two days.

### Retro- and Lentiviral Transduction

Retroviral transductions were performed as described (http://www.stanford.edu/group/nolan/retroviral systems/phx.html). Retroviral silencing of Fra-1 in RK3E cells was performed using the pRS-puro vector (Brummelkamp et al., 2002) with the following targeting sequences: sh-Fra-1(1) (TAACTAGCCTAGAACACTA) and sh-Fra-1(2) (GAAGTTCCACCTTGTGCCA). As negative control, pRS-puro without insert was used. RK3E cells were infected 4 times with viral supernatant and selected for puromycin resistance. We confirmed similar expression levels of TrkB and BNDF in all cell populations. Lentiviral transductions were performed as described previously (Ivanova et al., 2006). Silencing of Fra-1 in LM2 and MDA-MB-231 cells was performed using the following targeting sequences: sh-Fra-1(1) (GTAGATCCTTAGAGGTCCT) and sh-Fra-1(2) (GGCCTGTGCTTGAACCTGA). As negative control, vector without insert was used. Cells were infected once (2.10⁶ cells with 1,5.10⁷ viral particles) and GFP-positive cells were selected by fluorescence activated cell sorting (FACS).

### In Vivo Inoculation of Tumor Cells

All animal work was done in accordance with a protocol approved by the Institutional Animal Experiment Ethics Committee. Female Balb/c nude mice aged 6-8 weeks were used for all xenografting experiments. RK3E cells were injected sub-cutaneously (10⁵ viable cells in 150 μl PBS in each flank). Mice were sacrificed when the tumor length reached a size of 15 mm or when the tumors started to ulcerate. Tumor width (W) and length (L) were measured twice a week using a caliper and tumor volume was estimated using the formula (L·W²/2). LM2 cells were injected in the 4^{th} mammary fat pad of nude mice (10⁶ cells in 50 μl of a 1:1 mixture of PBS and growth factor-reduced Matrigel). Tumors were surgically removed after one month and mice were kept for 6 additional weeks. For experimental lung metastasis formation, MDA-MB-231 and LM2 cells were injected into the lateral tail vein (10⁶ or 10⁵ viable cells in 150 μl PBS). All animals were sacrificed three months or one month after injection, respectively.

### Quantification of Pulmonary Metastasis

Mice were sacrificed using CO₂ asphyxiation and the lungs were subsequently removed and dissected. Lungs were fixed in an Ethanol/Acetic acid/Formol saline fixative (EAF) and examined under a stereoscope. Macroscopic pulmonary metastases were identified as aberrant white masses on the surface of the lungs. For histological assessment of metastases, 8 sections from independent positions in the lungs were stained with hemoatoxylin-eosin (H&E) and the total number of metastases in these sections was determined. Alternatively, lungs were fixed in formaldehyde and imaged within 2 hours by fluorescence microscopy for quantification of the fluorescence emitted by GFP-labeled LM2 cells. Images were taken with the same intensities and exposure times, and the mean fluorescence intensity per surface area occupied by tumor cells was quantified using ImageJ software (http://rsb.info.nih.gov/ij/download.html) with the MBF plug-in bundle (http://www.macbiophotonics.ca/downloads.htm).

### In Vivo Analysis of Fra-1 Target Genes

Silencing of Fra-1-regulated genes in LM2 cells was performed using pLKO.1 vectors obtained from the Sigma Mission sh-RNA library. GFP-labelled LM2 cells were infected with lentivirus-expressing sh-RNAs, selected with puromycin for 2 days and injected in mice 7 days after lentiviral infection. Cells infected with an empty vector were used as a control. Female Balb/c nude mice 6-8 weeks of age were used for all xenografting experiments. LM2 cells were injected into the lateral tail vein (10⁵ viable cells in 150 μl PBS). Animals were sacrificed 5 weeks after injection using CO₂ asphyxiation and the lungs were subsequently dissected and imaged within 2 hours by fluorescence microscopy for quantification of the fluorescence emitted by GFP-labeled LM2 cells. Images were taken with the same intensities and exposure times, and the mean fluorescence intensity was quantified using ImageJ software (http://rsb.info.nih.gov/ij/download.html) with the MBF plug-in bundle (http://www.macbiophotonics.ca/downloads.htm). Fluorescence intensity observed in the lungs of mice injected with cells carrying sh-RNAs were normalized to the fluorescence intensity of the lungs of mice injected at the same time with control cells.

### Migration and Invasion Assays

RK3E clones (2,5.10⁵ cells/well) and MDA-MB-231 cells (3.10⁵ cells/well) were seeded in serum free medium into the upper well of BD BioCoat™ Control 8.0 μm PET Membrane 6-well Cell Culture Inserts for the migration assays, or BD BioCoat™ BD Matrigel™ Invasion Chamber, 8.0 μm PET Membrane 6-well Cell Culture Inserts for the invasion assays. Migration and invasion towards the lower well containing medium with 10% serum were assessed 24 hours later. Membranes were processed according to the manufacturer's recommendation. Migrating cells were stained with crystal violet and counted using bright-field microscopy (average number of cells on 8 fields at 100× magnification).

### Immunofluorescence

1.10⁵ cells were plated on collagen-coated Labtek slides (Nalge Nunc International) and left overnight in complete medium, washed in PBS, fixed in 4% PBS-buffered formaldehyde and processed for indirect immunofluorescence. Fra-1 antibody (1:200), E-cadherin antibody (1:200) and Alexa568-coupled phalloidin (A12380, Invitrogen; 1:200) were used.

### Immunohistochemistry

Histological sections and haematoxylin-eosin staining were performed using standard procedures. Paraffin sections were deparaffinized, rehydrated, pretreated in 0.1 mM sodium citrate pH 6.0, washed and incubated with peroxide. The tissue was incubated with primary antibodies for Fra-1 (1:200) or Ki-67 (1:4000). Secondary antibody was PowerVision+(DPVB+999HRP; ImmunoLogic). Peroxidase activity was detected with Liquid DAB (K3468; DAKO).

### SYBR-Green Real-Time RT-PCR

Total RNA was DNase-treated with RQ1 RNase-Free DNase (Promega). Reverse transcription was performed using Superscript II first strand kit (Invitrogen). qRT-PCR was performed with the SYBR Green PCR Master Mix (Applied Biosystems) on an ABI PRISM 7700 Sequence Detection System. The primer sets used were as follows: rat Fra-1: 5′-GCAGACACAGACAGTCCAG-3′ and 5′-CCATCCACTGCAATTCCTG-3′; rat HPRT1: 5′-CTGGTGAAAAGGACCTCTCG-3′ and 5′-TGAAGTGCTCATTATAGTCAAGGGCA-3′. mRNA levels were normalized using HPRT1 mRNA levels.

The primer sets used to detect Fra-1-regulated genes were as follows: human ABHD11: 5′-TTCAACTCCATCGCCAAGAT-3′ and 5′-CACCGTGGTTACGAGCATC-3′; human ADORA2B: 5′-TCTGTGTCCCGCTCAGGT-3′ and 5′-GATGCCAAAGGCAAGGAC-3′; human BIRC5: 5′-GCCCAGTGTTTCTTCTGCTT-3′ and 5′-CCGGACGAATGCTTTTTATG-3′; human CENPM: 5′-AACACGGCCACCATCTTG-3′ and 5′-GGGACTTTGCCAAGTGGAC-3′; human CHAF1A: 5′-GGAGAGGAGAGACGAGCAGA-3′ and 5′-CTTGCTCCCGTTCACATTG-3′; human CHML: 5′-TTATCTCCCACCAGGTTCCTC-3′ and 5′-TTCTCTTATTTCTTCTTTGAAGGTGAT-3′; human D21S2056E: 5′-GCAAGGCTGGGAAGAAAGA-3′ and 5′-GGGTGCAGGATCTCAGTCAT-3′; human E2F1: 5′-TCCAAGAACCACATCCAGTG-3′ and 5′-CTGGGTCAACCCCTCAAG-3′; human EZH2: 5′-TGGTCTCCCCTACAGCAGAA-3′ and 5′-TCATCTCCCATATAAGGAATGTTATG-3′; human FEN1: 5′-ACCCCGAACCAAGCTTTAG-3′ and 5′-GGGCCACATCAGCAATTAGT-3′; human H2AFZ: 5′-CACCGTGGGTCCGATTAG-3′ and 5′-GTCCTTTCCAGCCTTACCG-3′; human IGFBP3: 5′-AACGCTAGTGCCGTCAGC-3′ and 5′-CGGTCTTCCTCCGACTCAC-3′; human PAICS: 5′-TTTTCAGTTATTACAGGAAGCAGGT-3′ and 5′-TGAAAGCTGTCTCCCCACAT-3′; human PHLDA1: 5′-TCTGCACAAAAACTGGTGAGAC-3′ and 5′-ACTGCTCAGCCTGCCATC-3′; human PPP2R3A: 5′-CAGACTCCAGAGGTGATCAAGA-3′ and 5′-CGGGGACTACTTGGAGAGGT-3′; human PTGES: 5′-ACGCTGCTGGTCATCAAGA-3′ and 5′-TCTTCCGCAGCCTCACTT-3′; human PTP4A1: 5′-GGCCACAATCTTCAATGAGTAA-3′ and 5′-TGCTGTGCCTGGCAGTAA-3′; human SEC14L1: 5′-AGGGGCTGAGTGGTGATG-3′ and 5′-GTAGTCGGCATCTAGTTTGTCGT-3′; human SFN: 5′-CAGAGTCCGGCATTGGTC-3′ and 5′-GCTCTGGGGACACACAGG-3′; human SH3GL1: 5′-AGGAGGTGGCAGAAACCAG-3′ and 5′-TGACTCACCTGCTCGATGTC-3′; human TJAP1: 5′-AGAGCTGCCGACAAACAGAC-3′ and 5′-AGTCATTCTGGGAGGTGACG-3′; human TRFP: 5′-GGAACCCTGCGTTTCTACTG-3′ and 5′-ACAGGCATCTGGGACACAC-3′; human YTHDF1: 5′-CGACGACTTTGCTCACTACG-3′ and 5′-TTCGACTCTGCCGTTCCTT-3′; mRNA levels were normalized using beta-Actin mRNA levels.

### Western Blot Analysis

Western blotting was performed using standard procedures. We used goat antibodies to mouse (1706516, BioRad) and to rabbit (ALI0404, BioSource) conjugated with horseradish peroxidase as secondary antibodies, and developed the blots using ECL (Dura, Pierce)

### Gel Shift Experiments

Gel shift experiments were performed as previously described (Desmet et al., 2004). The sense strand of the AP-1 probe used had the following sequence: 5′-GGTTCGCTTGATGAGTCAGCCGGAA-3′. For supershift experiments, the nuclear extracts were pre-incubated with 2 μg of anti-Fra-1 antibody for 30 min

### Microarray Gene Expression Profiling

Full description of the methods for each experiment is available at http://www.ebi.ac.uk/microarray-as/aer/#ae-main[0] (accession numbers E-NCMF-20 and E-NCMF-21). Briefly, total RNA was isolated, purified and amplified. Amplified (a)RNA was subsequently labeled either with Cy5 or Cy3. Labeled aRNA was hybridized to oligo-arrays (Agilent 4× whole genome arrays for rat or human) and a dye-swap was performed for each experimental sample.

### Classifier Generation

We collected six publicly available datasets containing both raw gene expression microarray data of breast cancer samples and the corresponding information on distant metastasis-free survival and breast cancer specific survival. In order to avoid cross-platform discrepancies, the study was limited to Human Genome HGU-133A Affymetrix© arrays. The datasets were downloaded from NCBI's Gene Expression Omnibus (GEO, http://www.ncbi.nlm.nih.gov/geo/) with the following identifiers; GSE6532 (Loi et al., 2007), GSE3494 (Miller et al., 2005), GSE1456 (Pawitan et al., 2005), GSE7390 (Desmedt et al., 2007) and GSE5327 (Minn et al., 2005). The Chin et al. (Chin et al., 2006) data set was downloaded from ArrayExpress (http://www.ebi.ac.uk/, identifier E-TABM-158).

To ensure comparability between the different datasets, they were all subjected to the same pre-processing procedure. Microarray quality-control assessment was carried out using the R AffyPLM package, available from the Bioconductor web site (http://www.bioconductor.org.). We applied the Relative Log Expression (RLE) and Normalized Unscaled Standard Errors (NUSE) tests. Chip pseudo-images were produced to assess artefacts on arrays that failed to pass the preceding quality control tests. Approximately 1 to 5% of the arrays of the datasets did not pass the quality control tests. Selected arrays were normalized according to a 3-step procedure using the RMA expression measure algorithm (http://www.bioconductor.org): RMA background correction convolution, median centering of each gene across arrays separately for each data set and quantile normalization of all arrays. Out of the 947 unique collected microarray samples of sufficient quality, 509 had Distant Metastasis Free Survival (DMFS) data available. We employed these samples as training set, and will denote this sets as the ‘Affymetrix training set’. From the 947 samples, we also selected a separate validation set consisting of 388 samples for which breast cancer specific survival (BCSS) was available. We denote this set as the ‘Affymetrix validation set’. This set is completely non-overlapping in terms of samples with the Affymetrix training set, and is therefore a fully independent validation set.

The experimental Fra-1 signature was derived from the microarray analysis of Fra-1-depleted LM2 cells versus empty vector control cells. Probes that were significantly regulated in both sh-Fra-1 cell populations and in two independent microarray analyses were selected. This resulted in a set of 1140 probes, significantly regulated (p<10⁻⁸). The probes were converted to the corresponding probes on Affymetrix U133A arrays via Martview from BioMart (http://www.biomart.org/index.html). As this probe set contained multiple probes mapping to the same Entrez IDs, we selected a single Affymetrix© HGU-133A probe for each Entrez ID in the following manner. Probes were selected based on the Affymetrix algorithm probe extension, favoring ‘_at’ over ‘_x_at’ over ‘_s_at’. Expression of remaining duplicate probes were averaged. From this set of 1234 unique probes, probes were extracted that exhibited a significant p-value (p<0.05, log-rank test) on the Affymetrix training set. This resulted in a subset of 183 probes, the Fra-1 signature. We employed the hypergeometric test to determine whether a set of probes of this size and significantly associated with outcome, could have been selected from a randomly selected set of 1234 probes. Next, we employed the Affymetrix training set to define a nearest centroid classifier for these 183 probes. The ‘poor prognosis’ centroid was derived from the samples with a metastatic event before 60 months of follow-up. The ‘good prognosis’ centroid was derived from the samples with no metastatic event and a follow-up longer than 60 months.

For the validation of the Fra-1 classifier, we employed, in addition to the Affymetrix training set, the series of 295 breast cancer samples from the Netherlands Cancer Institute (van de Vijver et al., 2002). We refer to this set as ‘NKI295’. Each sample in the independent validation sets was assigned to the nearest centroid as determined by the highest Spearman rank order correlation score between the gene expression value of the corresponding probe se is of each sample and the centroid values of the ‘poor prognosis’ and ‘good prognosis’ centroid. For the validation of the Fra-1 classifier on the NKI295, the Rosetta© reporter IDs were mapped to the corresponding Entrez IDs. When multiple reporters mapped to the same Entrez ID, we selected the probe with the highest variance.

Survival analyses were performed using the Kaplan-Meier estimate of the survival function. Comparison between survival curves was performed using the log-rank test. Hazard ratios were estimated using a multivariate Cox proportional hazard model. The endpoints of these analyses were DMFS for the ‘Affymetrix training set and the NKI295 and BCSS for the Affymetrix validation set.

TABLE 1
Multivariate analysis of the Fra-1 classifier
and clinical variables on the NKI295.

| | | | 95% CI for Hazard Ratio | |
|---|---|---|---|---|
| | P-value | Hazard ratio | Lower | Upper |
| Grade | 9.6E−2 | 1.32 | 0.95 | 1.83 |
| LN | 3.3E−2 | 0.82 | 0.55 | 1.23 |
| ER | 5.5E−2 | 1.15 | 0.72 | 1.85 |
| Size | 1.4E−2 | 1.03 | 1.00 | 1.05 |
| Fra1-classifier | 7.4E−5 | 2.83 | 1.69 | 4.73 |

LN: lymph node status
ER: Estrogen receptor status

TABLE 2
169 genes with marked selection of the 32 genes

| | | Gene Description |
|---|---|---|
| | | SEQ ID NO (represents a human (homo sapiens) |
| | | cDNA sequence of said gene) |
| Classifier | | A list of these cDNA and corresponding amino acid |
| Gene ID | HGNC Symbol | sequences of these 169 genes is given after Table 5 |
| 1 | ABHD11 | abhydrolase domain containing 11 |
| | | SEQ ID NO: 1; SEQ ID NO: 170 |
| 2 | ACP6 | acid phosphatase 6, lysophosphatidic |
| | | SEQ ID NO: 33; SEQ ID NO: 202 |
| 3 | ACSL5 | acyl-CoA synthetase long-chain family member 5 |
| | | SEQ ID NO: 34; SEQ ID NO: 203 |
| 4 | ACTN1 | Actinin, alpha 1 |
| | | SEQ ID NO: 35; SEQ ID NO: 204 |
| 5 | ADORA2B | adenosine A2b receptor |
| | | SEQ ID NO: 2; SEQ ID NO: 171 |
| 6 | AES | amino-terminal enhancer of split |
| | | SEQ ID NO: 36; SEQ ID NO: 205 |
| 7 | AKT2 | v-akt murine thymoma viral oncogene homolog 2 |
| | | SEQ ID NO: 37; SEQ ID NO: 206 |
| 8 | ANAPC2 | anaphase promoting complex subunit 2 |
| | | SEQ ID NO: 38; SEQ ID NO: 207 |
| 9 | ANXA7 | annexin A7 |
| | | SEQ ID NO: 39; SEQ ID NO: 208 |
| 10 | APH1B | anterior pharynx defective 1 homolog B (C. elegans) |
| | | SEQ ID NO: 40; SEQ ID NO: 209 |
| 11 | ARL6IP5 | ADP-ribosylation-like factor 6 interacting protein 5 |
| | | SEQ ID NO: 41; SEQ ID NO: 210 |
| 12 | ARPC5 | actin related protein 2/3 complex, subunit 5, 16 kDa |
| | | SEQ ID NO: 42; SEQ ID NO: 211 |
| 13 | ATP1B1 | ATPase, Na+/K+ transporting, beta 1 polypeptide |
| | | SEQ ID NO: 43; SEQ ID NO: 212 |
| 14 | ATP9A | ATPase, Class II, type 9A |
| | | SEQ ID NO: 44; SEQ ID NO: 213 |
| 15 | AURKB | aurora kinase B |
| | | SEQ ID NO: 3; SEQ ID NO: 172 |
| 16 | B4GALT5 | UDP-Gal:betaGlcNAc beta 1,4-galactosyltransferase, |
| | | polypeptide 5 |
| | | SEQ ID NO: 45; SEQ ID NO: 214 |
| 17 | BECN1 | beclin 1 (coiled-coil, myosin-like BCL2 interacting protein) |
| | | SEQ ID NO: 46; SEQ ID NO: 215 |
| 18 | BIRC5 | baculoviral IAP repeat-containing 5 (survivin) |
| | | SEQ ID NO: 4; SEQ ID NO: 173 |
| 19 | BMP1 | bone morphogenetic protein 1 |
| | | SEQ ID NO: 47; SEQ ID NO: 216 |
| 20 | BTG1 | B-cell translocation gene 1, anti-proliferative |
| | | SEQ ID NO: 48; SEQ ID NO: 217 |
| 21 | C1orf144 | chromosome 1 open reading frame 144 |
| | | SEQ ID NO: 49; SEQ ID NO: 218 |
| 22 | C22orf18 | chromosome 22 open reading frame 18 |
| | | SEQ ID NO: 5; SEQ ID NO: 174 |
| 23 | CALU | Calumenin |
| | | SEQ ID NO: 50; SEQ ID NO: 219 |
| 24 | CASC3 | cancer susceptibility candidate 3 |
| | | SEQ ID NO: 51; SEQ ID NO: 220 |
| 25 | CASP1 | caspase 1, apoptosis-related cysteine protease (interleukin 1, |
| | | beta, convertase) |
| | | SEQ ID NO: 52; SEQ ID NO: 221 |
| 26 | CBPIN | CCNDBP1 interactor |
| | | SEQ ID NO: 53; SEQ ID NO: 222 |
| 27 | CD164 | CD164 antigen, sialomucin |
| | | SEQ ID NO: 54; SEQ ID NO: 223 |
| 28 | CD99 | CD99 antigen |
| | | SEQ ID NO: 55; SEQ ID NO: 224 |
| 29 | CDC42BPB | CDC42 binding protein kinase beta (DMPK-like) |
| | | SEQ ID NO: 56; SEQ ID NO: 225 |
| 30 | CELSR2 | cadherin, EGF LAG seven-pass G-type receptor 2 (flamingo |
| | | homolog, Drosophila) |
| | | SEQ ID NO: 57; SEQ ID NO: 226 |
| 31 | CGI-119 | CGI-119 protein |
| | | SEQ ID NO: 58; SEQ ID NO: 227 |
| 32 | CHAF1A | chromatin assembly factor 1, subunit A (p150) |
| | | SEQ ID NO: 6; SEQ ID NO: 175 |
| 33 | CHML | choroideremia-like (Rab escort protein 2) |
| | | SEQ ID NO: 7; SEQ ID NO: 176 |
| 34 | CIAPIN1 | cytokine induced apoptosis inhibitor 1 |
| | | SEQ ID NO: 59; SEQ ID NO: 228 |
| 35 | COL4A2 | collagen, type IV, alpha 2 |
| | | SEQ ID NO: 60; SEQ ID NO: 229 |
| 36 | COPB | coatomer protein complex, subunit beta |
| | | SEQ ID NO: 61; SEQ ID NO: 230 |
| 37 | CPT2 | carnitine palmitoyltransferase II |
| | | SEQ ID NO: 62; SEQ ID NO: 231 |
| 38 | CRIM1 | cysteine rich transmembrane BMP regulator 1 (chordin-like) |
| | | SEQ ID NO: 63; SEQ ID NO: 232 |
| 39 | CRYL1 | crystallin, lambda 1 |
| | | SEQ ID NO: 64; SEQ ID NO: 233 |
| 40 | CUGBP2 | CUG triplet repeat, RNA binding protein 2 |
| | | SEQ ID NO: 65; SEQ ID NO: 234 |
| 41 | CXorf6 | chromosome X open reading frame 6 |
| | | SEQ ID NO: 66; SEQ ID NO: 235 |
| 42 | CYBRD1 | cytochrome b reductase 1 |
| | | SEQ ID NO: 67; SEQ ID NO: 236 |
| 43 | D21S2056E | DNA segment on chromosome 21 (unique) 2056 expressed |
| | | sequence |
| | | SEQ ID NO: 8; SEQ ID NO: 177 |
| 44 | DCC1 | defective in sister chromatid cohesion homolog 1 (S. cerevisiae) |
| | | SEQ ID NO: 68; SEQ ID NO: 237 |
| 45 | DCTN6 | dynactin 6 |
| | | SEQ ID NO: 69; SEQ ID NO: 238 |
| 46 | DGCR8 | DiGeorge syndrome critical region gene 8 |
| | | SEQ ID NO: 70; SEQ ID NO: 239 |
| 47 | DNCLI2 | dynein, cytoplasmic, light intermediate polypeptide 2 |
| | | SEQ ID NO: 71; SEQ ID NO: 240 |
| 48 | DVL3 | dishevelled, dsh homolog 3 (Drosophila) |
| | | SEQ ID NO: 72; SEQ ID NO: 241 |
| 49 | DYRK2 | Dual-specificity tyrosine-(Y)-phosphorylation regulated kinase 2 |
| | | SEQ ID NO: 73; SEQ ID NO: 242 |
| 50 | DYSF | dysferlin, limb girdle muscular dystrophy 2B (autosomal |
| | | recessive) |
| | | SEQ ID NO: 74; SEQ ID NO: 243 |
| 51 | E2F1 | E2F transcription factor 1 |
| | | SEQ ID NO: 9; SEQ ID NO: 178 |
| 52 | EIF2S2 | eukaryotic translation initiation factor 2, subunit 2 beta, 38 kDa |
| | | SEQ ID NO: 75; SEQ ID NO: 244 |
| 53 | EIF4A2 | eukaryotic translation initiation factor 4A, isoform 2 |
| | | SEQ ID NO: 76; SEQ ID NO: 245 |
| 54 | EXT2 | exostoses (multiple) 2 |
| | | SEQ ID NO: 77; SEQ ID NO: 246 |
| 55 | EZH2 | enhancer of zeste homolog 2 ( [symbol] ) |
| | | SEQ ID NO: 10; SEQ ID NO: 179 |
| 56 | FAT4 | FAT tumor suppressor homolog 4 (Drosophila) |
| | | SEQ ID NO: 78; SEQ ID NO: 247 |
| 57 | FEN1 | flap structure-specific endonuclease 1 |
| | | SEQ ID NO: 11; SEQ ID NO: 180 |
| 58 | FLJ12529 | pre-mRNA cleavage factor I, 59 kDa subunit |
| | | SEQ ID NO: 79; SEQ ID NO: 248 |
| 59 | FLJ20364 | hypothetical protein FLJ20364 |
| | | SEQ ID NO: 80; SEQ ID NO: 249 |
| 60 | FNDC3A | fibronectin type III domain containing 3A |
| | | SEQ ID NO: 81; SEQ ID NO: 250 |
| 61 | FOSL1 | FOS-like antigen 1 |
| | | SEQ ID NO: 12; SEQ ID NO: 181 |
| 62 | FOXM1 | forkhead box M1 |
| | | SEQ ID NO: 13; SEQ ID NO: 182 |
| 63 | GCDH | glutaryl-Coenzyme A dehydrogenase |
| | | SEQ ID NO: 82; SEQ ID NO: 251 |
| 64 | GDF15 | growth differentiation factor 15 |
| | | SEQ ID NO: 83; SEQ ID NO: 252 |
| 65 | GLRX | glutaredoxin (thioltransferase) |
| | | SEQ ID NO: 84; SEQ ID NO: 253 |
| 66 | GOLT1B | golgi transport 1 homolog B (S. cerevisiae) |
| | | SEQ ID NO: 85; SEQ ID NO: 254 |
| 67 | H2AFZ | H2A histone family, member Z |
| | | SEQ ID NO: 14; SEQ ID NO: 183 |
| 68 | HUWE1 | HECT, UBA and WWE domain containing 1 |
| | | SEQ ID NO: 86; SEQ ID NO: 255 |
| 69 | ID1 | inhibitor of DNA binding 1, dominant negative helix-loop-helix |
| | | protein |
| | | SEQ ID NO: 87; SEQ ID NO: 256 |
| 70 | IDH3A | isocitrate dehydrogenase 3 (NAD+) alpha |
| | | SEQ ID NO: 88; SEQ ID NO: 257 |
| 71 | IFNGR1 | interferon gamma receptor 1 |
| | | SEQ ID NO: 89; SEQ ID NO: 258 |
| 72 | IFRG28 | 28 kD interferon responsive protein |
| | | SEQ ID NO: 90; SEQ ID NO: 259 |
| 73 | IGFBP3 | insulin-like growth factor binding protein 3 |
| | | SEQ ID NO: 15; SEQ ID NO: 184 |
| 74 | IL15 | interleukin 15 |
| | | SEQ ID NO: 91; SEQ ID NO: 260 |
| 75 | IMP-2 | IGF-II mRNA-binding protein 2 |
| | | SEQ ID NO: 92; SEQ ID NO: 261 |
| 76 | ITGA5 | integrin, alpha 5 (fibronectin receptor, alpha polypeptide) |
| | | SEQ ID NO: 93; SEQ ID NO: 262 |
| 77 | ITGB3 | integrin, beta 3 (platelet glycoprotein IIIa, antigen CD61) |
| | | SEQ ID NO: 94; SEQ ID NO: 263 |
| 78 | ITM1 | integral membrane protein 1 |
| | | SEQ ID NO: 95; SEQ ID NO: 264 |
| 79 | ITM2B | integral membrane protein 2B |
| | | SEQ ID NO: 96; SEQ ID NO: 265 |
| 80 | ITM2C | integral membrane protein 2C |
| | | SEQ ID NO: 97; SEQ ID NO: 266 |
| 81 | KIAA0182 | KIAA0182 protein |
| | | SEQ ID NO: 98; SEQ ID NO: 267 |
| 82 | KIAA1102 | KIAA1102 protein |
| | | SEQ ID NO: 99; SEQ ID NO: 268 |
| 83 | KLF4 | Kruppel-like factor 4 (gut) |
| | | SEQ ID NO: 100; SEQ ID NO: 269 |
| 84 | KLF6 | Kruppel-like factor 6 |
| | | SEQ ID NO: 101; SEQ ID NO: 270 |
| 85 | LEPROTL1 | leptin receptor overlapping transcript-like 1 |
| | | SEQ ID NO: 102; SEQ ID NO: 271 |
| 86 | LIMS1 | LIM and senescent cell antigen-like domains 1 |
| | | SEQ ID NO: 103; SEQ ID NO: 272 |
| 87 | LOC203069 | hypothetical protein LOC203069 |
| | | SEQ ID NO: 104; SEQ ID NO: 273 |
| 88 | LOC440085 | similar to prothymosin alpha |
| | | SEQ ID NO: 105; SEQ ID NO: 274 |
| 89 | LOXL2 | lysyl oxidase-like 2 |
| | | SEQ ID NO: 106; SEQ ID NO: 275 |
| 90 | LTBP3 | latent transforming growth factor beta binding protein 3 |
| | | SEQ ID NO: 107; SEQ ID NO: 276 |
| 91 | MAOA | monoamine oxidase A |
| | | SEQ ID NO: 108; SEQ ID NO: 277 |
| 92 | MAPKAPK3 | mitogen-activated protein kinase-activated protein kinase 3 |
| | | SEQ ID NO: 109; SEQ ID NO: 278 |
| 93 | MARCKS | myristoylated alanine-rich protein kinase C substrate |
| | | SEQ ID NO: 110; SEQ ID NO: 279 |
| 94 | MBD3 | methyl-CpG binding domain protein 3 |
| | | SEQ ID NO: 111; SEQ ID NO: 280 |
| 95 | MCM10 | MCM10 minichromosome maintenance deficient 10 |
| | | ( [symbol] ) |
| | | SEQ ID NO: 16; SEQ ID NO: 185 |
| 96 | MCM2 | MCM2 minichromosome maintenance deficient 2, mitotin |
| | | ( [symbol] ) |
| | | SEQ ID NO: 17; SEQ ID NO: 186 |
| 97 | MICAL2 | microtubule associated monoxygenase, calponin and LIM |
| | | domain containing 2 |
| | | SEQ ID NO: 112; SEQ ID NO: 281 |
| 98 | MTDH | Metadherin |
| | | SEQ ID NO: 18; SEQ ID NO: 187 |
| 99 | MYC | v-myc myelocytomatosis viral oncogene homolog (avian) |
| | | SEQ ID NO: 113; SEQ ID NO: 282 |
| 100 | MYO1F | myosin IF |
| | | SEQ ID NO: 114; SEQ ID NO: 283 |
| 101 | NF2 | neurofibromin 2 (bilateral acoustic neuroma) |
| | | SEQ ID NO: 115; SEQ ID NO: 284 |
| 102 | NINJ1 | ninjurin 1 |
| | | SEQ ID NO: 116; SEQ ID NO: 285 |
| 103 | NMD3 | NMD3 homolog (S. cerevisiae) |
| | | SEQ ID NO: 117; SEQ ID NO: 286 |
| 104 | NUAK1 | NUAK family, SNF1-like kinase, 1 |
| | | SEQ ID NO: 118; SEQ ID NO: 287 |
| 105 | NUP62 | nucleoporin 62 kDa |
| | | SEQ ID NO: 119; SEQ ID NO: 288 |
| 106 | P2RY11 | purinergic receptor P2Y, G-protein coupled, 11 |
| | | SEQ ID NO: 120; SEQ ID NO: 289 |
| 107 | P4HA2 | procollagen-proline, 2-oxoglutarate 4-dioxygenase (proline 4- |
| | | hydroxylase), alpha polypeptide II |
| | | SEQ ID NO: 121; SEQ ID NO: 290 |
| 108 | PAICS | phosphoribosylaminoimidazole carboxylase, |
| | | phosphoribosylaminoimidazole succinocarboxamide |
| | | synthetase |
| | | SEQ ID NO: 19; SEQ ID NO: 188 |
| 109 | PCOLN3 | procollagen (type III) N-endopeptidase |
| | | SEQ ID NO: 20; SEQ ID NO: 189 |
| 110 | PDCD4 | programmed cell death 4 (neoplastic transformation inhibitor) |
| | | SEQ ID NO: 122; SEQ ID NO: 291 |
| 111 | PDPK1 | 3-phosphoinositide dependent protein kinase-1 |
| | | SEQ ID NO: 123; SEQ ID NO: 292 |
| 112 | PFKM | phosphofructokinase, muscle |
| | | SEQ ID NO: 124; SEQ ID NO: 293 |
| 113 | PHLDA1 | pleckstrin homology-like domain, family A, member 1 |
| | | SEQ ID NO: 21; SEQ ID NO: 190 |
| 114 | PHLDA2 | pleckstrin homology-like domain, family A, member 2 |
| | | SEQ ID NO: 125; SEQ ID NO: 294 |
| 115 | PLSCR4 | phospholipid scramblase 4 |
| | | SEQ ID NO: 126; SEQ ID NO: 295 |
| 116 | PPP2R3A | protein phosphatase 2 (formerly 2A), regulatory subunit B″, |
| | | alpha |
| | | SEQ ID NO: 22; SEQ ID NO: 191 |
| 117 | PSMD7 | proteasome (prosome, macropain) 26S subunit, non-ATPase, 7 |
| | | (Mov34 homolog) |
| | | SEQ ID NO: 127; SEQ ID NO: 296 |
| 118 | PTBP1 | polypyrimidine tract binding protein 1 |
| | | SEQ ID NO: 128; SEQ ID NO: 297 |
| 119 | PTGES | prostaglandin E synthase |
| | | SEQ ID NO: 23; SEQ ID NO: 192 |
| 120 | PTK2 | PTK2 protein tyrosine kinase 2 |
| | | SEQ ID NO: 129; SEQ ID NO: 298 |
| 121 | PTP4A1 | protein tyrosine phosphatase type IVA, member 1 |
| | | SEQ ID NO: 24; SEQ ID NO: 193 |
| 122 | QKI | quaking homolog, KH domain RNA binding (mouse) |
| | | SEQ ID NO: 130; SEQ ID NO: 299 |
| 123 | RALA | v-ral simian leukemia viral oncogene homolog A (ras related) |
| | | SEQ ID NO: 131; SEQ ID NO: 300 |
| 124 | RARRES3 | retinoic acid receptor responder (tazarotene induced) 3 |
| | | SEQ ID NO: 132; SEQ ID NO: 301 |
| 125 | REST | RE1-silencing transcription factor |
| | | SEQ ID NO: 133; SEQ ID NO: 302 |
| 126 | RPL34 | ribosomal protein L34 |
| | | SEQ ID NO: 134; SEQ ID NO: 303 |
| 127 | RPL6 | ribosomal protein L6 |
| | | SEQ ID NO: 135; SEQ ID NO: 304 |
| 128 | S100A10 | S100 calcium binding protein A10 (annexin II ligand, calpactin |
| | | I, light polypeptide (p11)) |
| | | SEQ ID NO: 136; SEQ ID NO: 305 |
| 129 | S100P | S100 calcium binding protein P |
| | | SEQ ID NO: 137; SEQ ID NO: 306 |
| 130 | SCD | stearoyl-CoA desaturase (delta-9-desaturase) |
| | | SEQ ID NO: 25; SEQ ID NO: 194 |
| 131 | SCP2 | sterol carrier protein 2 |
| | | SEQ ID NO: 138; SEQ ID NO: 307 |
| 132 | SEC14L1 | SEC14-like 1 ( [symbol] ) |
| | | SEQ ID NO: 26; SEQ ID NO: 195 |
| 133 | SEC31L1 | SEC31-like 1 (S. cerevisiae) |
| | | SEQ ID NO: 139; SEQ ID NO: 308 |
| 134 | SEMA4C | sema domain, immunoglobulin domain (Ig), transmembrane |
| | | domain (TM) and short cytoplasmic domain, (semaphorin) 4C |
| | | SEQ ID NO: 140; SEQ ID NO: 309 |
| 135 | SERPINE1 | serine (or cysteine) proteinase inhibitor, clade E (nexin, |
| | | plasminogen activator inhibitor type 1), member 1 |
| | | SEQ ID NO: 141; SEQ ID NO: 310 |
| 136 | SFN | Stratifin |
| | | SEQ ID NO: 27; SEQ ID NO: 196 |
| 137 | SH3GL1 | SH3-domain GRB2-like 1 |
| | | SEQ ID NO: 28; SEQ ID NO: 197 |
| 138 | SIDT1 | SID1 transmembrane family, member 1 |
| | | SEQ ID NO: 142; SEQ ID NO: 311 |
| 139 | SLC35A1 | solute carrier family 35 (CMP-sialic acid transporter), member |
| | | A1 |
| | | SEQ ID NO: 143; SEQ ID NO: 312 |
| 140 | SLC35C1 | solute carrier family 35, member C1 |
| | | SEQ ID NO: 144; SEQ ID NO: 313 |
| 141 | SLC39A6 | solute carrier family 39 (zinc transporter), member 6 |
| | | SEQ ID NO: 145; SEQ ID NO: 314 |
| 142 | SLC4A7 | solute carrier family 4, sodium bicarbonate cotransporter, |
| | | member 7 |
| | | SEQ ID NO: 146; SEQ ID NO: 315 |
| 143 | SLC7A5 | solute carrier family 7 (cationic amino acid transporter, y+ |
| | | system), member 5 |
| | | SEQ ID NO: 147; SEQ ID NO: 316 |
| 144 | SMTN | Smoothelin |
| | | SEQ ID NO: 29; SEQ ID NO: 198 |
| 145 | STEAP1 | six transmembrane epithelial antigen of the prostate 1 |
| | | SEQ ID NO: 148; SEQ ID NO: 317 |
| 146 | TGFBI | transforming growth factor, beta-induced, 68 kDa |
| | | SEQ ID NO: 149; SEQ ID NO: 318 |
| 147 | TJAP1 | tight junction associated protein 1 (peripheral) |
| | | SEQ ID NO: 30; SEQ ID NO: 199 |
| 148 | TM2D3 | TM2 domain containing 3 |
| | | SEQ ID NO: 150; SEQ ID NO: 319 |
| 149 | TMEM59 | transmembrane protein 59 |
| | | SEQ ID NO: 151; SEQ ID NO: 320 |
| 150 | TMEM66 | transmembrane protein 66 |
| | | SEQ ID NO: 152; SEQ ID NO: 321 |
| 151 | TNRC6B | trinucleotide repeat containing 6B |
| | | SEQ ID NO: 153; SEQ ID NO: 322 |
| 152 | TPT1 | tumor protein, translationally-controlled 1 |
| | | SEQ ID NO: 154; SEQ ID NO: 323 |
| 153 | TRA2A | transformer-2 alpha |
| | | SEQ ID NO: 155; SEQ ID NO: 324 |
| 154 | TRFP | Trf (TATA binding protein-related factor)-proximal |
| | | homolog ( [symbol] ) SEQ ID NO: 31; SEQ ID NO: 200 |
| 155 | TRIB3 | tribbles homolog 3 (Drosophila) |
| | | SEQ ID NO: 156; SEQ ID NO: 325 |
| 156 | TSPAN15 | tetraspanin 15 |
| | | SEQ ID NO: 157; SEQ ID NO: 326 |
| 157 | TTC12 | tetratricopeptide repeat domain 12 |
| | | SEQ ID NO: 158; SEQ ID NO: 327 |
| 158 | TUSC4 | tumor suppressor candidate 4 |
| | | SEQ ID NO: 159; SEQ ID NO: 328 |
| 159 | UBE1L | ubiquitin-activating enzyme E1-like |
| | | SEQ ID NO: 160; SEQ ID NO: 329 |
| 160 | UBE2A | ubiquitin-conjugating enzyme E2A (RAD6 homolog) |
| | | SEQ ID NO: 161; SEQ ID NO: 330 |
| 161 | UBTF | upstream binding transcription factor, RNA polymerase I |
| | | SEQ ID NO: 162; SEQ ID NO: 331 |
| 162 | UCP2 | uncoupling protein 2 (mitochondrial, proton carrier) |
| | | SEQ ID NO: 163; SEQ ID NO: 332 |
| 163 | VDAC3 | voltage-dependent anion channel 3 |
| | | SEQ ID NO: 164; SEQ ID NO: 333 |
| 164 | VPS13B | vacuolar protein sorting 13B (yeast) |
| | | SEQ ID NO: 165; SEQ ID NO: 334 |
| 165 | WHSC1L1 | Wolf-Hirschhorn syndrome candidate 1-like 1 |
| | | SEQ ID NO: 166; SEQ ID NO: 335 |
| 166 | YIF1A | Yip1 interacting factor homolog A (S. cerevisiae) |
| | | SEQ ID NO: 167; SEQ ID NO: 336 |
| 167 | YTHDF1 | YTH domain family, member 1 |
| | | SEQ ID NO: 32; SEQ ID NO: 201 |
| 168 | ZNF207 | zinc finger protein 207 |
| | | SEQ ID NO: 168; SEQ ID NO: 337 |
| 169 | ZNF395 | zinc finger protein 395 |
| | | SEQ ID NO: 169; SEQ ID NO: 338 |

Genes common to both the classifiers generated from LM2 and MDA-MB-231 cells silenced for Fra1 expression

Genes with a higher centroid value in poor prognosis patients compared to good prognosis patients and that may be amenable drug targets are highlighted in bold. Fra1 (FOSL1) has also been included to this list.

TABLE 3
32 genes for which inhibitors are being claimed

| | Entrez | | Function |
|---|---|---|---|
| Symbol | ID | Gene Description | SEQ ID NO |
| ABHD11 | 83451 | abhydrolase domain | Hydrolase |
| | | containing 11 | SEQ ID NO :1 |
| ADORA2B | 136 | adenosine A2b receptor | G coupled receptor activity |
| | | | SEQ ID NO: 2 |
| AURKB | 9212 | aurora kinase B | protein serine/threonine kinase activity, |
| | | | transferase activity |
| | | | SEQ ID NO: 3 |
| BIRC5 | 332 | baculoviral IAP repeat- | caspase inhibitor activity, peptidase inhibitor |
| | | containing 5 (survivin) | activity |
| | | | SEQ ID NO: 4 |
| C22orf18 | 79019 | chromosome 22 open | condensed chromosome kinetochore |
| | | reading frame 18 | (component only) |
| | | | SEQ ID NO: 5 |
| CHAF1A | 10036 | chromatin assembly | chromatin binding |
| | | factor 1, subunit A (p150) | SEQ ID NO: 6 |
| CHML | 1122 | choroideremia-like (Rab | GTPase activiator activity |
| | | escort protein 2) | SEQ ID NO: 7 |
| D21S2056E | 8568 | DNA segment on | rRNA processing, component of |
| | | chromosome 21 (unique) | nucleus/nucleolus |
| | | 2056 expressed sequence | SEQ ID NO: 8 |
| E2F1 | 1869 | E2F transcription factor 1 | transcription activator/transcription |
| | | | corepressor/transcription factor activity |
| | | | SEQ ID NO: 9 |
| EZH2 | 2146 | enhancer of zeste | methyl transferase/transferase activity |
| | | homolog 2 (Drosophila) | SEQ ID NO: 10 |
| FEN1 | 2237 | flap structure-specific | 5′flap endonuclease/5′-3′ exonuclease activity, |
| | | endonuclease 1 | hydrolyase activity |
| | | | SEQ ID NO: 11 |
| FOSL1 | 8061 | FOS-like antigen 1 | transcription activator/transcription factor |
| | | | activity |
| | | | SEQ ID NO: 12 |
| FOXM1 | 2305 | forkhead box M1 | transcription factor activity |
| | | | SEQ ID NO: 13 |
| H2AFZ | 3015 | H2A histone family, | DNA binding |
| | | member Z | SEQ ID NO: 14 |
| IGFBP3 | 3486 | insulin-like growth factor | insulin like growth factor I binding, protein |
| | | binding protein 3 | tyrosine phosphatase activity |
| | | | SEQ ID NO: 15 |
| MCM10 | 55388 | MCM10 | protein binding, metal ion binding |
| | | minichromosome | SEQ ID NO: 16 |
| | | maintenance deficient 10 | |
| | | (S. cerevisiae) | |
| MCM2 | 4171 | MCM2 minichromosome | ATP binding/DNA binding/DNA replication |
| | | maintenance deficient 2, | origin binding/protein binding |
| | | mitotin (S. cerevisiae) | SEQ ID NO: 17 |
| MTDH | 92140 | metadherin | NF-kappaB binding/protein binding |
| | | | SEQ ID NO: 18 |
| PAICS | 10606 | phosphoribosylaminoimidazole | ATP binding/ligase, lyase activity |
| | | carboxylase, | SEQ ID NO: 19 |
| | | phosphoribosylaminoimidazole | |
| | | succinocarboxamide | |
| | | synthetase | |
| PCOLN3 | 5119 | procollagen (type III) N- | metallopeptidase activity, zinc ion binding |
| | | endopeptidase | SEQ ID NO: 20 |
| PHLDA1 | 22822 | pleckstrin homology-like | protein binding/apoptosis |
| | | domain, family A, member 1 | SEQ ID NO: 21 |
| PPP2R3A | 5523 | protein phosphatase 2 | protein phosphatase 2A regulator activity, |
| | | (formerly 2A), regulatory | protein binding activity |
| | | subunit B″, alpha | SEQ ID NO: 22 |
| PTGES | 9536 | prostaglandin E synthase | isomerase activity, prostaglandinE synthase |
| | | | activity |
| | | | SEQ ID NO: 23 |
| PTP4A1 | 7803 | protein tyrosine | hydrolase/protein tyrosine phosphatase |
| | | phosphatase type IVA, | activity |
| | | member 1 | SEQ ID NO: 24 |
| SCD | 6319 | stearoyl-CoA desaturase | oxidoreductase activity/iron ion binding |
| | | (delta-9-desaturase) | SEQ ID NO: 25 |
| SEC14L1 | 6397 | SEC14-like 1 | intracellular transport system |
| | | (S. cerevisiae) | SEQ ID NO: 26 |
| SFN | 2810 | stratifin | protein kinase C inhibitor |
| | | | SEQ ID NO: 27 |
| SH3GL1 | 6455 | SH3-domain GRB2-like 1 | lipid binding/protein binding |
| | | | SEQ ID NO: 28 |
| SMTN | 6525 | smoothelin | actin binding/structural constituent of muscle |
| | | | SEQ ID NO: 29 |
| TJAP1 | 93643 | tight junction associated | protein binding |
| | | protein 1 (peripheral) | SEQ ID NO: 30 |
| TRFP | 9477 | Trf (TATA binding | RNA polymerase II transcription mediator/ |
| | | protein-related factor)- | RNA polymerase activity, protein binding |
| | | proximal homolog | SEQ ID NO: 31 |
| | | (Drosophila) | |
| YTHDF1 | 54915 | YTH domain family, | Unknown |
| | | member 1 | SEQ ID NO: 32 |

TABLE 4
known inhibitors from genes of Table 3

| | Whole name/ | | |
|---|---|---|---|
| Gene | Protein | Inhibitors | Description |
| AURKB | Aurora kinase B | AZD1152 | selective inhibitor |
| | | | http://www.selleckchem.com/Product.asp?ClassID=46 |
| | | Hesperadin | small molecule, Boehringer Ingelheim, |
| | | (HESP) | http://www.boehringer-ingelheim.com |
| | | Z(M/W)447439 | small molecule |
| | | | http://www.selleckchem.com/Product.asp?ClassID=46 |
| | | VX-680 | http://www.selleckchem.com/Product.asp?ClassID=46 |
| | | PHA739358 | http://www.selleckchem.com/Product.asp?ClassID=46 |
| | | MLN8054 | http://www.selleckchem.com/Product.asp?ClassID=46 |
| ADORA2B | Adenosine A2b | PSB1115 | all and a few more are available at |
| | receptor | | http://www.tocris.com/and www.biocompare.com |
| | | CGS15943 | |
| | | DPCPX | |
| | | PSB601 | specific antagonist |
| | | SCH58261 | |
| | | 7-Chloro-4-hydroxy- | |
| | | 2-phenyl-1,8- | |
| | | naphthyridine* | |
| | | CGS-15953* | |
| BIRC5 | Survivin | LY2181308 | antisense molecule, specific (Eli Lilliy and Company, |
| | | (ISIS23722) | Indianapolis, IN) |
| | | YM155 | transcriptional repressor, in clinical development by |
| | | | Astellas Pharma, Inc. |
| | | EM1421 | transcriptional represser, Erimos Pharmaceuiticals |
| | | (Terameprocol) | |
| | | SPC3042 | antisense molecule designed by Hansen, Fisker, |
| | | | Westergaar et al. 2008 |
| | | EZN3042 | developed by Enzon Pharmaceuticals and Santaris |
| | | | Pharma Advances |
| | | Oxaliplatin | |
| E2F1 | E2F transcription | Mitoxantrane | alters the consensus DNA binding site (also works for |
| | factor 1 | | Sp1) |
| | | Distamycin | specifically inhibits E2F1-DNA complex |
| | | MGT-6a | microgonotropen |
| FOXM1 | Forkhead box M1 | Siomycin A | thiazole antibiotic |
| | | MG115 | proteosome inhibitor (www.biocompare.com) |
| | | MG132 | proteosome inhibitor (www.tocris.com) |
| | | Bortezomib | proteosome inhibitor |
| | | Thiostrepton | thiazole antibiotic (www.tocris.com) |
| PCOLN3 | procollagen | TIMP3 | also inhibits MMPs, ADAMs, ADAMTS4, 5 and |
| | (type III) | | VEGF-VEGFR interaction (www.biocompare.com) |
| | N-endopeptidase | | |
| | (ADAMTS-2) | α2-macroglobulin | http://www.enzolifesciences.com/BML- |
| | | | SE502/alpha2-macroglobulin-human-purified/ |
| PPP2R3A | protein phosphatase2 | Calyculin A | high potency for PP2A, low potency for PP1 |
| | (2A), regulatory | | |
| | subunit B, alpha | | |
| | (PP2A) | Cantharidic acid | inhibits PP2A and PP1 (for use in protein purification) |
| | | Candharidin | selective inhibitor of PP2A |
| | | Endothall | intermediate potency for PP2A |
| | | Microcystin LR | more potent for PP2A when compared to PP1 |
| | | Okadaic acid | completely inhibits PP2A at 1 nM (less potent for PP1) |
| | | Toutomycin | more potent for PP1, less for PP2A |
| | | Fostriecin sodium | potent inhibitors for PP2A and PP4 |
| | | salt | |
| PTP4A1 | Protein tyrosine | Pentamidine | inhibits all PRLs |
| | phosphatase type | | |
| | IVA, member 1 | | |
| | (PRL1) | | |
| SCD | Stearoyl-CoA | 4a (CVT-11563) | Koltun, Vasilevich, Parkhill, 2009 |
| | desaturase | | |
| | (delta-9- desaturase) | CVT-11127 | |
| | | 9-thiastearate | |
| SFN | Stratifin | R18 peptide | isoform independent inhibitor, antagonist |
| | (14-3-3 sigma) | | |
| EZH2 | Enhancer of zeste | 3-Deazaneplanocin | also induces SUZ12 degradation |
| | homolog 2 | A (DZNep) | |
| | | Isoliquiritigenin | |

*In an in vitro drug screen with the LOPAC library, we identified those two ADORA2B inhibitors as being selectively cytotoxic for breast cancer cells expressing high levels of Fra-1.

TABLE 5
clustering of the 32 genes from which inhibitors are being claimed

| | | SEQ ID NO of the |
|---|---|---|
| Cluster | Gene name | human cDNA |
| Enzymes | ABHD11, AURKB, CHML, | 1, 3, 7, 10, |
| | EZH2, FEN1, IGFBP3, | 11, 15, 19, 20, |
| | PAICS, PCOLN3, PPP2R3A, | 22, 23, 24,25 |
| | PTGES, PTP4A1, SCD | |
| Transcription factor | E2F1, FOSL1, FOXM1 | 9, 12, 13 |
| Structural proteins | C22orf18, CHAF1A, H2AFZ, | 5, 6, 14, 29, |
| | SMTN, TJAP1, D21S2056E | 30, 8 |
| receptor | ADORA2B | 2 |
| Adhesion molecule | MTDH | 18 |
| Apoptose inhibitor | BIRC5, PHLDA1 | 4, 21 |
| DNAreplication/ | MCM10, MCM2, TRFP | 16, 17, 31 |
| transcription | | |
| Remaining genes: | SEC14L1, SFN, SH3GL1, | 26, 27, 28, 32 |
| no cluster | YTHDF1 | |

Underlined genes are preferred.

TABLE 6
12 Fra-1-regulated genes essential for metastasis

| HGNC Symbol | Gene Description |
|---|---|
| ABHD11 | abhydrolase domain containing 11 |
| ADORA2B | adenosine A2b receptor |
| D21S2056E | DNA segment on chromosome 21 (unique) 2056 |
| | expressed sequence (also known as ribosomal |
| | RNA processing 1 homolog (RRP1) (S. cerevisiae); |
| | NNP-1; NOP52; RRP1A) |
| E2F1 | E2F transcription factor 1 |
| EZH2 | enhancer of zeste homolog 2 |
| IGFBP3 | insulin-like growth factor binding protein 3 |
| PAICS | phosphoribosylaminoimidazole carboxylase, |
| | phosphoribosylaminoimidazole |
| | succinocarboxamide synthetase |
| PPP2R3A | protein phosphatase 2 (formerly 2A), regulatory |
| | subunit B″, alpha |
| PTGES | prostaglandin E synthase |
| PTP4A1 | protein tyrosine phosphatase type IVA, member 1 |
| SFN | stratifin |
| SH3GL1 | SH3-domain GRB2-like 1 |

Twelve genes for which shRNA-mediated silencing in LM2 cells significantly inhibited lung metastasis formation.

### REFERENCES

Adiseshaiah, P., Lindner, D. J., Kalvakolanu, D. V., and Reddy, S. P. (2007). FRA-1 Proto-Oncogene Induces Lung Epithelial Cell Invasion and Anchorage-Independent Growth In vitro, but Is Insufficient to Promote Tumor Growth In vivo. Cancer Res 67, 6204-6211.
Adjei, A. A., Cohen, R. B., Franklin, W., Morris, C., Wilson, D., Molina, J. R., Hanson, L. J., Gore, L., Chow, L., Leong, S., et al. (2008). Phase I Pharmacokinetic and Pharmacodynamic Study of the Oral, Small-Molecule Mitogen-Activated Protein Kinase Kinase ½ Inhibitor AZD6244 (ARRY-142886) in Patients With Advanced Cancers. J Clin Oncol 26, 2139-2146.
Andersen, H., Mejlvang, J., Mahmood, S., Gromova, I., Gromov, P., Lukanidin, E., Kriajevska, M., Mellon, J. K., and Tulchinsky, E. (2005) Immediate and Delayed Effects of E-Cadherin Inhibition on Gene Regulation and Cell Motility in Human Epidermoid Carcinoma Cells. Mol Cell Biol 25, 9138-9150.
Ansieau, S., Bastid, J., Doreau, A., Morel, A.-P., Bouchet, B. P., Thomas, C., Fauvet, F., Puisieux, I., Doglioni, C., Piccinin, S., et al. (2008). Induction of EMT by Twist Proteins as a Collateral Effect of Tumor-Promoting Inactivation of Premature Senescence. Cancer Cell 14, 79-89.
Belguise, K., Kersual, N., Galtier, F., and Chalbos, D. (2005). FRA-1 expression level regulates proliferation and invasiveness of breast cancer cells. Oncogene 24, 1434-1444.
Bernards, R., and Weinberg, R. A. (2002). Metastasis genes: A progression puzzle. Nature 418, 823-823.
Blamey, R. W., Ellis, I. O., Pinder, S. E., Lee, A. H. S., Macmillan, R. D., Morgan, D. A. L., Robertson, J. F. R., Mitchell, M. J., Ball, G. R., Haybittle, J. L., and Elston, C. W. (2007). Survival of invasive breast cancer according to the Nottingham Prognostic Index in cases diagnosed in 1990-1999. European Journal of Cancer 43, 1548-1555.
Brummelkamp, T. R., Bernards, R., and Agami, R. (2002). Stable suppression of tumorigenicity by virus-mediated RNA interference. Cancer Cell 2, 243-247.
Carter, B. D., Zirrgiebel, U., and Barde, Y.-A. (1995). Differential Regulation of p21ras Activation in Neurons by Nerve Growth Factor and Brain-derived Neurotrophic Factor. J Biol Chem 270, 21751-21757.
Cavallaro, U., and Christofori, G. (2004). Cell adhesion and signalling by cadherins and Ig-CAMs in cancer. Nat Rev Cancer 4, 118-132.
Chin, K., DeVries, S., Fridlyand, J., Spellman, P. T., Roydasgupta, R., Kuo, W.-L., Lapuk, A., Neve, R. M., Qian, Z., Ryder, T., et al. (2006). Genomic and transcriptional aberrations linked to breast cancer pathophysiologies. Cancer Cell 10, 529-541.
Christofori, G. (2006). New signals from the invasive front. Nature 441, 444-450.
Desmedt, C., Piette, F., Loi, S., Wang, Y., Lallemand, F., Haibe-Kains, B., Viale, G., Delorenzi, M., Zhang, Y., d'Assignies, M. S., et al. (2007). Strong Time Dependence of the 76-Gene Prognostic Signature for Node-Negative Breast Cancer Patients in the TRANSBIG Multicenter Independent Validation Series. Clin Cancer Res 13, 3207-3214.
Desmet, C., Gosset, P., Pajak, B., Cataldo, D., Bentires-Alj, M., Lekeux, P., and Bureau, F. (2004). Selective Blockade of NF-{kappa}B Activity in Airway Immune Cells Inhibits the Effector Phase of Experimental Asthma. J Immunol 173, 5766-5775.
Douma, S., van Laar, T., Zevenhoven, J., Meuwissen, R., van Garderen, E., and Peeper, D. S. (2004). Suppression of anoikis and induction of metastasis by the neurotrophic receptor TrkB. Nature 430, 1034-1039.
Dunkler, D., Michiels, S., and Schemper, M. (2007). Gene expression profiling: Does it add predictive accuracy to clinical characteristics in cancer prognosis? European Journal of Cancer 43, 745-751.
Eferl, R., and Wagner, E. F. (2003). AP-1: a double-edged sword in tumorigenesis. Nat Rev Cancer 3, 859-868.
Eifel, P., Axelson, J. A., Costa, J., Crowley, J., Curran, W. J. J., Deshler, A., Fulton, S., Hendricks, C. B., Kemeny, M., Kornblith, A. B., et al. (2001). National Institutes of Health Consensus Development Conference Statement: Adjuvant Therapy for Breast Cancer, Nov. 1-3, 2000. J Natl Cancer Inst 93, 979-989.
Fidler, I. J. (2003). The pathogenesis of cancer metastasis: the ‘seed and soil’ hypothesis revisited. Nat Rev Cancer 3, 453-458.
Geiger, T. R., and Peeper, D. S. (2005). The Neurotrophic Receptor TrkB in Anoikis Resistance and Metastasis: A Perspective. Cancer Res 65, 7033-7036.
Goldhirsch, A., Wood, W. C., Gelber, R. D., Coates, A. S., Thurlimann, B., Senn, H. J., and Panel, M. (2007). Progress and promise: highlights of the international expert consensus on the primary therapy of early breast cancer 2007. Ann Oncol 18, 1133-1144.
Gupta, G. P., and Massague, J. (2006). Cancer Metastasis: Building a Framework. Cell 127, 679-695.
Hu, G., Chong, R. A., Yang, Q., Wei, Y., Blanco, M. A., Li, F., Reiss, M., Au, J. L., Haffty, B. G. and Kang, Y. (2009). MTDH activation by 8q22 genomic gain promotes chemoresistance and metastasis of poor-prognosis breast cancer. Cancer Cell 15, 9-20.
Hugo, H., Ackland, M. L., Blick, T., Lawrence, M. G., Clements, J. A., Williams, E. D., and Thompson, E. W. (2007). Epithelial-mesenchymal and mesenchymal-epithelial transitions in carcinoma progression. Journal of Cellular Physiology 213, 374-383.
Ivanova, N., Dobrin, R., Lu, R., Kotenko, I., Levorse, J., DeCoste, C., Schafer, X., Lun, Y., and Lemischka, I. R. (2006). Dissecting self-renewal in stem cells with RNA interference. Nature 442, 533-538.
Liotta, L. A., and Kohn, E. (2004). Anoikis: Cancer and the homeless cell. Nature 430, 973-974.
Loi, S., Haibe-Kains, B., Desmedt, C., Lallemand, F., Tutt, A. M., Gillet, C., Ellis, P., Harris, A., Bergh, J., Foekens, J. A., et al. (2007). Definition of Clinically Distinct Molecular Subtypes in Estrogen Receptor-Positive Breast Carcinomas Through Genomic Grade. J Clin Oncol 25, 1239-1246.
Lusa, L., Miceli, R., and Mariani, L. (2007). Estimation of predictive accuracy in survival analysis using R and S-PLUS. Computer Methods and Programs in Biomedicine 87, 132-137.
Mani, S. A., Guo, W., Liao, M.-J., Eaton, E. N., Ayyanan, A., Zhou, A. Y., Brooks, M., Reinhard, F., Zhang, C. C., Shipitsin, M., et al. (2008). The Epithelial-Mesenchymal Transition Generates Cells with Properties of Stem Cells. Cell 133, 704-715.
Milde-Langosch, K. (2005). The Fos family of transcription factors and their role in tumourigenesis. European Journal of Cancer 41, 2449-2461.
Miller, L. D., Smeds, J., George, J., Vega, V. B., Vergara, L., Ploner, A., Pawitan, Y., Hall, P., Klaar, S., Liu, E. T., and Bergh, J. (2005). An expression signature for p53 status in human breast cancer predicts mutation status, transcriptional effects, and patient survival. PNAS 102, 13550-13555.
Minn, A. J., Gupta, G. P., Siegel, P. M., Bos, P. D., Shu, W., Giri, D. D., Viale, A., Olshen, A. B., Gerald, W. L., and Massague, J. (2005). Genes that mediate breast cancer metastasis to lung. Nature 436, 518-524.
Ozanne, B. W., Spence, H. J., McGarry, L. C., and Hennigan, R. F. (2006). Transcription factors control invasion: AP-1 the first among equals. Oncogene 26, 1-10.
Pawitan, Y., Bjohle, J., Amler, L., Borg, A.-L., Egyhazi, S., Hall, P., Han, X., Holmberg, L., Huang, F., Klaar, S., et al. (2005). Gene expression profiling spares early breast cancer patients from adjuvant therapy: derived and validated in two population-based cohorts. Breast Cancer Research 7, R953-R964.
Ramaswamy, S., Ross, K. N., Lander, E. S., and Golub, T. R. (2003). A molecular signature of metastasis in primary solid tumors. Nat Genet. 33, 49-54.
Ramos-Nino, M. E., Timblin, C. R., and Mossman, B. T. (2002). Mesothelial Cell Transformation Requires Increased AP-1 Binding Activity and ERK-dependent Fra-1 Expression. Cancer Res 62, 6065-6069.
Ravdin, P. M., Siminoff, L. A., Davis, G. J., Mercer, M. B., Hewlett, J., Gerson, N., and Parker, H. L. (2001). Computer Program to Assist in Making Decisions About Adjuvant Therapy for Women With Early Breast Cancer. J Clin Oncol 19, 980-991.
Schemper, M., and Henderson, R. (2000). Predictive accuracy and explained variation in Cox regression. Biometrics 56, 249-255.
Thiery, J. P., and Sleeman, J. P. (2006). Complex networks orchestrate epithelial-mesenchymal transitions. Nat Rev Mol Cell Biol 7, 131-142.
Vallone, D., Battista, S., Pierantoni, G. M., Fedele, M., Casalino, L., Santoro, M., Viglietto, G., Fusco, A., and Verde, P. (1997). Neoplastic transformation of rat thyroid cells requires the junB and fra-1 gene induction which is dependent on the HMGI-C gene product. EMBO J. 16, 5310-5321.
van 't Veer, L. J., and Bernards, R. (2008). Enabling personalized cancer medicine through analysis of gene-expression patterns. Nature 452, 564-570.
van de Vijver, M. J., He, Y. D., van 't Veer, L. J., Dai, H., Hart, A. A. M., Voskuil, D. W., Schreiber, G. J., Peterse, J. L., Roberts, C., Marton, M. J., et al. (2002). A Gene-Expression Signature as a Predictor of Survival in Breast Cancer. N Engl J Med 347, 1999-2009.
Vial, E., Sahai, E., and Marshall, C. J. (2003). ERK-MAPK signaling coordinately regulates activity of Rac1 and RhoA for tumor cell motility. Cancer Cell 4, 67-79.
Yang, J., Mani, S. A., Donaher, J. L., Ramaswamy, S., Itzykson, R. A., Come, C., Savagner, P., Gitelman, I., Richardson, A., and Weinberg, R. A. (2004). Twist, a Master Regulator of Morphogenesis, Plays an Essential Role in Tumor Metastasis. Cell 117, 927-939.
Yang, J., and Weinberg, R. A. (2008). Epithelial-Mesenchymal Transition: At the Crossroads of Development and Tumor Metastasis. Developmental Cell 14, 818-829.
Zajchowski, D. A., Bartholdi, M. F., Gong, Y., Webster, L., Liu, H.-L., Munishkin, A., Beauheim, C., Harvey, S., Ethier, S. P., and Johnson, P. H. (2001). Identification of Gene Expression Profiles That Predict the Aggressive Behavior of Breast Cancer Cells. Cancer Res 61, 5168-5178.

1. A method for preventing, delaying and/or treating metastasis in a cancer patient comprising the administration of an inhibitor of a polypeptide, said polypeptide comprising an amino acid sequence that is encoded by a nucleotide sequence wherein the nucleotide sequence is selected from the groups consisting of:
(1) a nucleotide sequence encoding an enzyme PAICS, ABHD11, AURKB, CHML, EZH2, FEN1, IGFBP3, PCOLN3, PPP2R3A, PTGES, PTP4A1, and SCD,
(2) a nucleotide sequence encoding a transcription factor E2F1, FOSL1, and FOXM1,
(3) a nucleotide sequence encoding a structural protein C22orf18, CHAF1A, H2AFZ, SMTN, TJAP1, D21S2056E,
(4) a nucleotide sequence encoding a receptor ADORA2B;
(5) a nucleotide sequence encoding an adhesion molecule MTDH,
(6) a nucleotide sequence encoding an apoptose inhibitor BIRC5 and PHLDA1
(7) a nucleotide sequence encoding a protein involved in DNA replication/transcription MCM10, MCM2 and TRFP and
(8) a nucleotide sequence encoding a SEC14L1, SFN, SH3GL1 and YTHDF1.

2. A method according to claim 1, wherein the nucleotide sequence is selected from:
(a) a nucleotide sequence that has at least 60% identity with a sequence selected from SEQ ID NO: 19, 1, 2, 3, 4, 5, 6, 7, 8, 9, 10, 11, 12, 13, 14, 15, 16, 17, 18, 20, 21, 22, 23, 24, 25, 26, 27, 28, 29, 30, 31, 32; and,
(b) a nucleotide sequence that encodes an amino acid sequence that has at least 60% amino acid identity with an amino acid sequence encoded by a nucleotide sequence selected from SEQ ID NO; 19, 1, 2, 3, 4, 5, 6, 7, 8, 9, 10, 11, 12, 13, 14, 15, 16, 17, 18, 20, 21, 22, 23, 24, 25, 26, 27, 28, 29, 30, 31, 32.

3. A method inhibitor according to claim 1, wherein the inhibitor is a DNA or RNA molecule, a dominant negative, an inhibiting antibody raised against said polypeptide.

4. A method according to claim 3, wherein the DNA molecule is a nucleic acid construct which comprises a nucleotide sequence encoding an RNAi agent that is capable of inhibiting the expression of a polypeptide that comprises an amino acid sequence that is encoded by a nucleotide sequence selected from:
(a) a nucleotide sequence that has at least 60% identity with a sequence selected from SEQ ID NO: 1-32; and,
(b) a nucleotide sequence that encodes an amino acid sequence that has at least 60% amino acid identity with an amino acid sequence encoded by a nucleotide sequence selected from SEQ ID NO; 1-32;
and, wherein optionally the nucleotide sequence encoding the RNAi agent is operably linked to a promoter that is capable of driving expression of the nucleotide sequence in a cell.

5. A method according to claim 4, wherein in the nucleic acid construct the nucleotide sequence is selected from:
(a) a nucleotide sequence that has at least 60% identity with SEQ ID NO: 1, 2, 3, 7, 10, 11, 12, 15, 19, 20, 22, 23, 24, 25 and
(b) a nucleotide sequence that encodes an amino acid sequence that has at least 60% amino acid identity with an amino acid sequence encoded by the nucleotide sequence SEQ ID NO:1, 2, 3, 7, 10, 11, 12, 15, 19, 20, 22, 23, 24, 25.

6. Use of an inhibitor as defined in claim 1 for the manufacture of a medicament for preventing, delaying and/or treating metastasis in a cancer patient.

7. An inhibitor as defined in any one of claims 1 for preventing, delaying and/or treating metastasis in a cancer patient.

8. A method according to claim 1, wherein the method comprises the step of administering to the patient a therapeutically effective amount of a pharmaceutical composition comprising a nucleic acid construct as defined in claim 4 or 5 and preferably wherein the pharmaceutical composition is administered to a tumor cell of a cancer patient to be treated.

9. A method for identification of a substance capable of preventing, delaying and/or treating metastasis in a cancer patient, the method comprising the steps of:
(a) providing a test cell population capable of expressing a nucleotide sequence as present in a nucleic acid construct, wherein said nucleotide sequence is a nucleotide sequence that has at least 60% identity with a sequence selected from SEQ ID NO: 1-32 as identified in claim 1 or SEQ ID NO:1-169; and,
a nucleotide sequence that encodes an amino acid sequence that has at least 60% amino acid identity with an amino acid sequence encoded by a nucleotide sequence selected from SEQ ID NO; 1-32 or SEQ ID NO:1-169;
(b) contacting the test cell population with the substance;
(c) determining the expression level of the nucleotide sequence or the activity or steady state level of the polypeptide in the test cell population contacted with the substance;
(d) comparing the expression, activity or steady state level determined in (c) with the expression, activity or steady state level of the nucleotide sequence or of the polypeptide in a test cell population that is not contacted with the substance; and,
(e) identifying a substance that produces a difference in expression level, activity or steady state level of the nucleotide sequence or the polypeptide, between the test cell population that is contacted with the substance and the test cell population that is not contacted with the substance.

10. An ex vivo method of prognosticating metastasis in a cancer patient, preferably a breast cancer patient, comprising identifying differential modulation of a gene (relative to the expression of a same gene in a control) in a combination of genes selected from the groups consisting of genes represented by the following sequences SEQ ID NO:1-32 as identified in claim 1 or SEQ ID NO:1-169 and optionally using this result to decide about the treatment to be given to the patient.

11. An ex vivo method of prognosticating the absence of metastasis in a cancer patient, preferably a breast cancer patient, comprising identifying a lack of differential modulation of a gene (relative to the expression of a same gene in a control population) in a combination of genes selected from the groups consisting of genes represented by the following sequences SEQ ID NO:1-32 as identified in claim 1 or SEQ ID NO:1-169 and optionally using this result to decide about the treatment to be given to the patient.

12. A method according to claim 11, wherein said prognosis of the absence of metastasis is for a five year period.

13. A diagnostic portfolio comprising or consisting of isolated nucleic acid sequences, their complement or portions thereof, of a combination of genes selected from the groups consisting of genes represented by the following sequences SEQ ID NO:1-32 or SEQ ID NO:1-169.

14. A kit for prognosticating metastasis in a cancer patient comprising reagents for detecting nucleic acid sequences, their complements or portions thereof in a combination of genes selected from the groups consisting of genes represented by the following sequences SEQ ID NO:1-32 or SEQ ID NO:1-169, and optionally further comprising instructions.

15. A kit according to claim 14 further comprising reagents for conducting a microarray analysis and optionally further comprising a medium through which said nucleic acid sequences or their complements are assayed, preferably wherein said medium is a microarray
